(19) United States
(12) Patent Application Publication
(10) Pub. No.: US 2026/0130896 A1
(43) Pub. Date: May 14, 2026

(54) COMPOSITIONS AND METHODS FOR DELIVERY OF OPHTHALMOLOGICAL ACTIVES

(71) Applicant: ADS Therapeutics LLC, Irvine, CA (US)

(72) Inventors: Van DINH, Irvine, CA (US); Wenkui Ken FANG, Irvine, CA (US); Jinsong NI, Irvine, CA (US); Rong YANG, Irvine, CA (US)

(21) Appl. No.: 19/441,283

(22) Filed: Jan. 6, 2026

Related U.S. Application Data
(63) Continuation of application No. PCT/US2024/040714, filed on Aug. 2, 2024.
(60) Provisional application No. 63/530,919, filed on Aug. 4, 2023.
(60) Provisional application No. 63/596,913, filed on Nov. 7, 2023.
(60) Provisional application No. 63/631,841, filed on Apr. 9, 2024.
(60) Provisional application No. 63/632,214, filed on Apr. 10, 2024.
(60) Provisional application No. 63/658,306, filed on Jun. 10, 2024.
(60) Provisional application No. 63/667,874, filed on Jul. 5, 2024.

(51) Int. Cl. A61K 31/439; A61K 9/00; A61K 47/14; A61P 27/10

(52) U.S. Cl. CPC A61K 31/439; A61K 9/0048; A61K 47/14; A61P 27/10

(57) ABSTRACT

The present application provides compositions, methods, and dispensers for topical delivery of ophthalmological active pharmaceutical ingredients (APIs).

In one example, a composition is provided comprising an active pharmaceutical ingredient soluble in MCT wherein the API is not atropine or a salt thereof, a medium chain triglyceride (MCT); and a semi-fluorinated alkane compound. In another example, a nano-emulsion is provided comprising about an active pharmaceutical ingredient soluble in MCT; a medium chain triglyceride (MCT); and a semi-fluorinated alkane compound, wherein the nano-emulsion has a droplet particle size D90 of less than about 100 nm. Dispensers containing the compositions are also provided and include glass and polyethylene terephthalate dispensers or containers. Methods of using the compositions are also provided and include a method for treating an ocular condition in a subject comprising administering the compositions or nano-emulsions to an eye of the subject.

The present application also provides compositions and methods for treating ophthalmological conditions such as presbyopia and glaucoma. The compositions can comprise a muscarinic cholinergic receptor agonist and a semi-fluorinated alkane compound; or can comprise a muscarinic cholinergic receptor agonist, a semi-fluorinated alkane compound, and an organic cosolvent. The compositions can confer chemical stability of the muscarinic cholinergic receptor agonist.

### CROSS-REFERENCE TO RELATED APPLICATIONS

This application claims the benefit of priority to U.S. Provisional Patent Application No. 63/667,874, filed on Jul. 5, 2024, U.S. Provisional Patent Application No. 63/658,306, filed on Jun. 10, 2024, U.S. Provisional Patent Application No. 63/632,214, filed on Apr. 10, 2024, U.S. Provisional Patent Application No. 63/631,841, filed on Apr. 9, 2024, U.S. Provisional Patent Application No. 63/596,913, filed on Nov. 7, 2023, and U.S. Provisional Patent Application No. 63/530,919, filed on Aug. 4, 2023, the contents of each of which are hereby incorporated by reference in their entireties.

### TECHNICAL FIELD

This disclosure relates to compositions for topical delivery of ophthalmological active pharmaceutical ingredients (APIs), and methods for making and using the compositions.

This disclosure also relates to pharmaceutical compositions comprising a muscarinic cholinergic receptor agonist and a semi-fluorinated alkane compound. This disclosure also relates to methods of treatment of ophthalmological diseases such as presbyopia using the compositions.

### BACKGROUND

Various attempts have been made to formulate active pharmaceutical ingredients compounds (APIs) for ocular delivery, e.g., for treating and preventing conditions in the eye or improving symptoms thereof. However, challenges with delivery formulations can limit the usability of the APIs, their activity and shelf life, and can in some instances result in systemic side effects. In common practice, APIs for ophthalmological are often formulated in aqueous formulations, however, many APIs can degrade in aqueous formulations. Sometimes adjustments to pH can slow the degradation of APIs in aqueous formulations, but such pH adjustments can result in ocular irritation and discomfort for the user. There remains a need for improved compositions for ophthalmological delivery of APIs.

Presbyopia is an ophthalmological condition in which adults develop difficulty seeing close objects clearly. The condition develops with age and is most common in adults 45 years or older. It is believed that a decrease in the flexibility of the eye lens over time disrupts the proper focusing of light on the retina, causing blurriness. Current treatments involve eyeglasses or contact lenses, lens implant surgery, or refractive surgery. Some compounds are under investigation for potential pharmaceutical treatment of presbyopia.

### SUMMARY

This disclosure relates to compositions for topical delivery of ophthalmological active pharmaceutical ingredients (APIs), comprising medium chain triglycerides (MCT), a semi-fluorinated alkane (SFA), and an active pharmaceutical ingredient (API), and methods for making and using the compositions, such as methods for treating or slowing the progression of, or reducing one or more symptoms of, an ocular condition in a subject. Also provided herein are dispensers or containers comprising the compositions.

The compositions can, in some instances, be non-aqueous.

Applicant has surprisingly discovered that compositions comprising medium chain triglycerides (MCT) and a semi-fluorinated alkane (SFA) can act as an advantageous delivery system for ophthalmological APIs that are soluble in MCT, resulting in ocularly tolerable formulation with increased stability and long-term shelf life of the API in the formulations, and improved tissue distribution and retention time of the API.

Applicant has further surprisingly discovered that compositions comprising MCT, SFA, and an API can be made into nano-emulsions having a droplet size D90 of less than 100 nm, less than 50 nm, less than 20 nm, less than 15 nm, less than 10 nm, and even less than 5 nm.

Applicant has further surprisingly discovered that compositions comprising MCT, SFA, and an API can, in some instances, exhibit incompatibility or lesser compatibility with certain materials used for storage and delivery containers or can have improved shelf life and stability of the API when stored or delivered in certain materials as compared to certain other materials.

This disclosure is based, at least in part, on a surprising realization that any API soluble in MCT can be effectively delivered to ocular tissues using the compositions described herein, and further that in some embodiments, the compositions can result in improved stability and shelf life for the API. Without being bound by theory, it is believed that the MCT provides increased solubility of the API for delivery in a formulation comprising a SFA. Without being bound by theory, it is further believed the SFA assists in maintaining a non-irritating, and in some embodiments, non-aqueous formulation for delivery of the API. This disclosure is also based on the surprising realization that, in some instances described herein, it is possible to create nanoemulsions of the compositions having droplet size D90 of less than 100 nm, less than 10 nm, and even less than 5 nm. Without being bound by theory, it is believed that a nano-emulsion (e.g., small droplet size) comprising MCT and SFA leads to improved long-term stability of the API in the formulation as compared to aqueous formulations, as well as increased availability of the API, improved tissue penetration and distribution of the API, and improved residence time in ocular tissues. This disclosure is further based on the surprising realization that, in some instances of the compositions described herein, the compositions can be incompatible with or have lesser shelf life and API stability when stored in containers of certain materials.

This disclosure provides compositions comprising medium chain triglycerides (MCT), a semi-fluorinated alkane (SFA), and an active pharmaceutical ingredient (API) soluble in MCT, and methods for making and using the compositions. The compositions described herein can provide improved delivery formulations for ocular or ophthalmological APIs including compositions with improved ocular tolerability, improved API stability, improved shelf life of the compositions and the API, an improved ocular tissue distribution and retention time, while limiting the systemic distribution and therefore systemic effect of such APIs.

In one aspect, described herein is a composition comprising:
about 0.001% to about 20% (w/w) active pharmaceutical ingredient (API), wherein the API has a solubility in MCT of at least from about 0.01% to about 10% (w/w), and wherein the API is not atropine or a salt thereof,
from about 1% to about 50% (w/w) of a medium chain triglyceride (MCT); and
from about 50% to about 99% (w/w) of a semi-fluorinated alkane compound.

In this and other embodiments, the compositions can optionally have the following features. The composition can be non-aqueous. The semi-fluorinated alkane compound can be selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10). The MCT can be present in a concentration of from about 10% to about 50% (w/w) and the semi-fluorinated alkane compound is present in a concentration of from about 50% to about 90% (w/w). The MCT can be present in a concentration of from about 10% to about 30% (w/w) and the semi-fluorinated alkane compound is present in a concentration of from about 70% to about 90% (w/w). The API can be present in a concentration of from about 0.01% to 1% (w/w). The API can be present in a free base form. The MCT can be a triglyceride of fatty acids and the fatty acids can comprise hexanoic acid, octanoic acid, decanoic acid, dodecanoic acid or combinations of two or more thereof. The MCT can be present in a concentration of about 5% (w/w) and the semi-fluorinated alkane compound can be present in a concentration of about 95% (w/w), the MCT can be present in a concentration of about 10% (w/w) and the semi-fluorinated alkane compound can be present in a concentration of about 90% (w/w), the MCT can be present in a concentration of about 15% (w/w) and the semi-fluorinated alkane compound can be present in a concentration of about 85% (w/w), or the MCT can be present in a concentration of about 20% (w/w) and the semi-fluorinated alkane compound can be present in a concentration of about 80% (w/w). The semi-fluorinated alkane can be perfluorohexyloctane (F6H8). The MCT can be a triglyceride of fatty acids and the fatty acids can consist essentially of hexanoic acid, octanoic acid, decanoic acid, dodecanoic acid or combinations thereof. The topical ophthalmological composition can be an emulsion. The API in the topical ophthalmological composition can be chemically stable for at least 6 months, at least 1 year, at least 18 months, at least 2 years or longer. The API can be selected from alpha agonists, antibiotics, corticosteroids, antinicotinic agents, antiglaucoma agents, antihistamines, antivirals, cycloplegics/mydriatics, mast cell stabilizers, miotics, ophthalmic NSAIDs, cyclosporine, riboflavin 5′-phosphate ophthalmic compounds, and VEGF inhibitors and VEGF receptor inhibitors.

In some embodiments, the API is not a muscarinic receptor antagonist.

In another aspect, described herein is a composition consisting essentially of:
about 0.001% to about 5% (w/w) active pharmaceutical ingredient (API), wherein the API has a solubility in MCT of at least from about 0.01% to about 10% (w/w), and wherein the API is not atropine or a salt thereof,
from about 1% to about 50% (w/w) of a medium chain triglyceride (MCT); and
from about 50% to about 99% (w/w) of a semi-fluorinated alkane compound.

In this and other embodiments, the compositions can have the following optional features. The semi-fluorinated alkane compound can be selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10). The MCT can be present in a concentration of about 5% (w/w) and the semi-fluorinated alkane compound can be present in a concentration of about 95% (w/w), wherein the MCT can be present in a concentration of about 10% (w/w) and the semi-fluorinated alkane compound can be present in a concentration of about 90% (w/w), wherein the MCT can be present in a concentration of about 15% (w/w) and the semi-fluorinated alkane compound can be present in a concentration of about 85% (w/w), or wherein the MCT can be present in a concentration of about 20% (w/w) and the semi-fluorinated alkane compound can be present in a concentration of about 80% (w/w).

In another aspect, described herein is a composition comprising:
about 0.01% to about 1% (w/w) active pharmaceutical ingredient (API), wherein the API has a solubility in MCT of at least from about 0.01% to about 10% (w/w), and wherein the API is not atropine or a salt thereof,
about 10% to about 30% (w/w) medium chain triglyceride (MCT); and
about 70% to about 90% (w/w) semi-fluorinated alkane compound selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).

In another aspect, described herein is a topical ophthalmological nano-emulsion comprising:
about 0.001% to about 5% (w/w) active pharmaceutical ingredient (API), wherein the API has a solubility in MCT of at least from about 0.01% to about 10% (w/w);
from about 1% to about 50% (w/w) of a medium chain triglyceride (MCT); and
from about 50% to about 99% (w/w) of a semi-fluorinated alkane compound,

wherein the nano-emulsion has a droplet particle size D90 of less than about 100 nm.

In these and other embodiments, the compositions can optionally have the following features. The nano-emulsion can have a droplet particle size D90 of less than about 50 nm, less than about 20 nm, less than about 10 nm, or less than about 5 nm, or has a droplet particle size D90 of from about 2 to about 3 nm. The nano-emulsion can have a peak droplet particle size of less than about 10 nm or can have a peak droplet particle size of from about 1 nm to about 5 nm. The composition can be non-aqueous. The semi-fluorinated alkane compound can be selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10). The MCT can be present in a concentration of from about 10% to about 50% (w/w) and the semi-fluorinated alkane compound is present in a concentration of from about 50% to about 90% (w/w). The MCT can be present in a concentration of from about 10% to about 30% (w/w) and the semi-fluorinated alkane compound is present in a concentration of from about 70% to about 90% (w/w). The API can be present in a concentration of from about 0.01% to 1% (w/w). The API can be present in a free base form. The MCT can be a triglyceride of fatty acids and the fatty acids can comprise hexanoic acid, octanoic acid, decanoic acid, dodecanoic acid or combinations of two or more thereof. The MCT can be present in a concentration of about 5% (w/w) and the semi-fluorinated alkane compound can be present in a concentration of about 95% (w/w), the MCT can be present in a concentration of about 10% (w/w) and the semi-fluorinated alkane compound can be present in a concentration of about 90% (w/w), the MCT can be present in a concentration of about 15% (w/w) and the semi-fluorinated alkane compound can be present in a concentration of about 85% (w/w), or the MCT can be present in a concentration of about 20% (w/w) and the semi-fluorinated alkane compound can be present in a concentration of about 80% (w/w). The semi-fluorinated alkane can be perfluorohexyloctane (F6H8). The MCT can be a triglyceride of fatty acids and the fatty acids can consist essentially of hexanoic acid, octanoic acid, decanoic acid, dodecanoic acid or combinations thereof. The topical ophthalmological composition can be an emulsion. The API in the topical ophthalmological composition can be chemically stable for at least 6 months, at least 1 year, at least 18 months, at least 2 years or longer. The API can be selected from alpha agonists, antibiotics, corticosteroids, anticholinergic agents, antiglaucoma agents, antihistamines, antivirals, cycloplegics/mydriatics, mast cell stabilizers, miotics, ophthalmic NSAIDs, cyclosporine, riboflavin 5′-phosphate ophthalmic compounds, and VEGF inhibitors and VEGF receptor inhibitors. The API can be a muscarinic receptor antagonist. The API can be atropine, an atropine mimetic or analog, or a free base or salt form thereof. The API can be atropine free base. The API can be atropine, an atropine mimetic or analog, or a free base or salt form thereof and is present in a concentration of from about 0.001% to about 1% (w/w).

In some instances, the API is optionally not a muscarinic receptor antagonist. In some instances, the API is optionally not atropine, an atropine mimetic or analog, or a free base or salt form thereof.

In another aspect, described herein is a topical ophthalmological nano-emulsion consisting essentially of:
about 0.001% to about 5% (w/w) active pharmaceutical ingredient (API), wherein the API has a solubility in MCT of at least from about 0.01% to about 10% (w/w);
from about 1% to about 50% (w/w) of a medium chain triglyceride (MCT); and
from about 50% to about 99% (w/w) of a semi-fluorinated alkane compound,

wherein the nano-emulsion has a droplet particle size D90 of less than about 100 nm, less than about 50 nm, less than about 20 nm, less than about 10 nm, less than about 5 nm, or from about 2 to about 3 nm.

In these and other embodiments, the topical ophthalmological nano-emulsions can have the following optional features. The semi-fluorinated alkane compound can be selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10). The MCT can be present in a concentration of about 5% (w/w) and the semi-fluorinated alkane compound can be present in a concentration of about 95% (w/w), wherein the MCT can be present in a concentration of about 10% (w/w) and the semi-fluorinated alkane compound can be present in a concentration of about 90% (w/w), wherein the MCT can be present in a concentration of about 15% (w/w) and the semi-fluorinated alkane compound can be present in a concentration of about 85% (w/w), or wherein the MCT can be present in a concentration of about 20% (w/w) and the semi-fluorinated alkane compound can be present in a concentration of about 80% (w/w). The API can be a muscarinic receptor antagonist. The API can be atropine, an atropine mimetic or analog, or a free base or salt form thereof. The API can be atropine free base. The API can be atropine, an atropine mimetic or analog, or a free base or salt form thereof and is present in a concentration of from about 0.001% to about 1% (w/w).

In some instances, the API is optionally not a muscarinic receptor antagonist. In some instances, the API is optionally not atropine, an atropine mimetic or analog, or a free base or salt form thereof.

In another aspect, described herein is a topical ophthalmological nano-emulsion comprising:
about 0.001% to about 1% (w/w) active pharmaceutical ingredient (API), wherein the API has a solubility in MCT of at least from about 0.01% to about 10% (w/w);
about 10% to about 30% (w/w) medium chain triglyceride (MCT); and
about 70% to about 90% (w/w) semi-fluorinated alkane compound selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10),

wherein the nano-emulsion has a droplet particle size D90 of less than about 100 nm, less than about 20 nm, less than about 10 nm, less than about 5 nm, or from about 2 to about 3 nm.

In these and other embodiments, the topical ophthalmological nano-emulsions can have the following optional features. The API can be a muscarinic receptor antagonist. The API can be atropine, an atropine mimetic or analog, or a free base or salt form thereof. The API can be atropine free base. The API can be atropine, an atropine mimetic or analog, or a free base or salt form thereof and is present in a concentration of from about 0.001% to about 1% (w/w).

In some instances, the API is optionally not a muscarinic receptor antagonist. In some instances, the API is optionally not atropine, an atropine mimetic or analog, or a free base or salt form thereof.

In another aspect, described herein is a dispenser comprising:
a container portion comprising an interior wall defining an interior volume and adapted to contact and store a liquid within at least a portion of the interior volume;
a dispensing portion adapted to dispense one or more drops of a liquid out of the container portion; and
any one of the compositions of claims 1-19 or the nano-emulsions of claims 20-45, or a composition comprising:
   about 0.001% to about 5% (w/w) active pharmaceutical ingredient (API), wherein the API has a solubility in MCT of at least from about 0.01% to about 10% (w/w)
from about 1% to about 50% (w/w) of a medium chain triglyceride (MCT); and
from about 50% to about 99% (w/w) of a semi-fluorinated alkane compound, contained within the interior volume.

In these and other embodiments, the dispenser can have the following optional features. The interior wall can comprise less than about 10% of one or more of low density polyethylene (LDPE) or polypropylene (PP). The interior wall can optionally not comprise low density polyethylene (LDPE) or polypropylene (PP). The interior wall can comprise glass or polyethylene terephthalate. The interior wall can consist essentially of glass or polyethylene terephthalate (PET). The concentration of the API can remain more than 90% of the initial API concentration after storage in the dispenser for a period of at least 6 months, or the concentration of the API can remain more than 90% of the initial API concentration after storage in the dispenser for a period of at least 12 months or the concentration of the API can remain more than 90% of the initial API concentration after storage in the dispenser for at least a period of 24 months. The container portion and the dispensing portion each, independently, can consist essentially of glass or polyethylene terephthalate (PET). The volume of the one or more drops each independently can be from about 5 μl to about 40 μl. The composition in the dispenser can comprise:
from about 0.001% to about 1% (w/w) atropine, an atropine mimetic or analog, or a free base or salt form thereof,
about 5% to about 30% (w/w) medium chain triglyceride (MCT); and
about 70% to about 95% (w/w) semi-fluorinated alkane compound selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).

In another aspect, described herein is a method for treating, slowing the progression of, or reducing one or more symptoms of an ocular condition in a subject comprising administering the topical ophthalmological compositions or the topical ophthalmological nano-emulsions described herein to an eye of the subject. In these and other embodiments, the ocular condition can optionally be selected from neurotrophic keratitis, glaucoma, elevated intraocular pressure, ocular hypertension, presbyopia, myopia, ocular rosacea, dry eye disease, meibomian gland dysfunction, blepharitis, allergic conjunctivitis, atopic keratoconjunctivitis, vernal keratoconjunctivitis, pterygium, pinguecula, corneal transplant rejection, graft versus host disease, ocular allergy, uveitis, anterior uveitis, Behcet's disease, Sjogren's syndrome, Stevens-Johnson syndrome, ocular cicatricial pemphigoid, chronic ocular surface inflammation caused by viral infection, herpes simplex keratitis, atopic conjunctivitis, Lyell's syndrome, neovascularization induced by viral, bacterial, fungal, or parasitic infection, contact lens induced neovascularization, ulceration, alkali burns, stem cell deficiency, neovascular glaucoma, Steven Johnson syndrome, tumor in the eye, aphakia, pseudophakia, astigmatism, blepharospasm, cataract, conjunctival diseases, corneal diseases, corneal ulcer, eyelid diseases, lacrimal apparatus diseases, pupil disorders, refractive disorders, strabismus.

In another aspect, described herein is a method for delivering an API to an eye of a subject comprising administering any one of the topical ophthalmological compositions or the topical ophthalmological nano-emulsions described herein to the eye of the subject. In these and other embodiments, the API can optionally be selected from alpha agonists, antibiotics, corticosteroids, anticholinergic agents, antiglaucoma agents, antihistamines, antivirals, cycloplegics/mydriatics, mast cell stabilizers, miotics, ophthalmic NSAIDs, cyclosporine, riboflavin 5′-phosphate ophthalmic compounds, and VEGF inhibitors and VEGF receptor inhibitors.

In another aspect, described herein is a method for slowing myopia progression in a subject or for relieving vitreous floater symptoms in a subject, comprising administering anyone of the nano-emulsions described herein, to an eye of the subject, wherein the API is a muscarinic receptor antagonist. In these and other embodiments, the API can optionally be selected from atropine, an atropine mimetic or analog, or a free base or salt form thereof.

In some instances of the foregoing methods for treating, slowing the progression of, or reducing one or more symptoms of an ocular condition, methods for delivering an API to an eye of a subject, and methods for slowing myopia progression in a subject or for relieving vitreous floater symptoms in a subject, the topical ophthalmological composition or the nano-emulsion is absorbed transcorneally in the eye. In some instances, the topical ophthalmological composition or the nano-emulsion is selectively absorbed transcorneally in the eye as compared to periorbitally. In some instances, the topical ophthalmological composition is non-aqueous. In some instances, the topical ophthalmological composition is an emulsion. In some instances, the topical ophthalmological composition is non-aqueous, and wherein the topical ophthalmological composition or the nano-emulsion is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API (e.g., used for treating, slowing the progression of, or reducing one or more symptoms of the ocular condition, for delivering an API to an eye of a subject, for slowing myopia progression in a subject, or for relieving vitreous floater symptoms in a subject. In some instances, the topical ophthalmological composition or the nano-emulsion is administered to the subject every about 6 to about 8 hours or three times a day. In some instances, the topical ophthalmological composition or the nano-emulsion is administered to the subject once a day. In some instances, the topical ophthalmological composition or the nano-emulsion is administered to the subject twice a day. In some instances, the API is in a free base form.

In another aspect, described herein is a method for treating, slowing the progression of, or reducing one or more symptoms of an ocular condition in a subject comprising administering a topical ophthalmological composition to an eye of the subject, wherein the topical ophthalmological composition comprises: about 0.001% to about 5% (w/w) API, wherein the API has a solubility in MCT of at least from about 0.01% to about 10% (w/w), and wherein the API is atropine; from about 1% to about 50% (w/w) of an MCT; and from about 50% to about 99% (w/w) of a semi-fluorinated alkane compound. In some instances, the topical ophthalmological composition comprises about 0.02% (w/w) API. In some instances, the topical ophthalmological composition comprises about 0.04% (w/w) API. In some instances, the topical ophthalmological composition comprises about 0.05% (w/w) API. In some instances, the topical ophthalmological composition comprises about 1% (w/w) API. In some instances, the topical ophthalmological composition comprises about 5% to about 20% (w/w) MCT. In some instances, the topical ophthalmological composition is administered to the eye of the subject every about 6 to about 8 hours or three times a day.

In some instances, the topical ophthalmological composition is administered to the subject once a day. In some instances, the topical ophthalmological composition is administered to the subject twice a day. In some instances, the topical ophthalmological composition is non-aqueous. In some instances, the topical ophthalmological composition is non-aqueous and the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, or reducing one or more symptoms of the ocular condition. In some instances, the API is in a free base form. In some instances, the ocular condition is selected from neurotrophic keratitis, glaucoma, elevated intraocular pressure, ocular hypertension, presbyopia, myopia, ocular rosacea, dry eye disease, meibomian gland dysfunction, blepharitis, allergic conjunctivitis, atopic keratoconjunctivitis, vernal keratoconjunctivitis, pterygium, pinguecula, corneal transplant rejection, graft versus host disease, ocular allergy, uveitis, anterior uveitis, Behcet's disease, Sjogren's syndrome, Stevens-Johnson syndrome, ocular cicatricial pemphigoid, chronic ocular surface inflammation caused by viral infection, herpes simplex keratitis, atopic conjunctivitis, Lyell's syndrome, neovascularization induced by viral, bacterial, fungal, or parasitic infection, contact lens induced neovascularization, ulceration, alkali burns, stem cell deficiency, neovascular glaucoma, Steven Johnson syndrome, tumor in the eye, aphakia, pseudophakia, astigmatism, blepharospasm, cataract, conjunctival diseases, corneal diseases, corneal ulcer, eyelid diseases, lacrimal apparatus diseases, pupil disorders, refractive disorders, strabismus. In some instances, the topical ophthalmological composition is absorbed transcorneally in the eye. In some instances, the topical ophthalmological composition is selectively absorbed transcorneally in the eye as compared to periorbitally.

In another aspect, described herein is a method of delivering an API to an eye of a subject comprising administering to the eye of the subject a topical ophthalmological composition, wherein the topical ophthalmological composition comprises: about 0.001% to about 5% (w/w) API, wherein the API has a solubility in MCT of at least from about 0.01% to about 10% (w/w), and wherein the API is atropine; from about 1% to about 50% (w/w) of an MCT; and from about 50% to about 99% (w/w) of a semi-fluorinated alkane compound. In some instances, the topical ophthalmological composition comprises about 0.02% (w/w) API. In some instances, the topical ophthalmological composition comprises about 0.04% (w/w) API. In some instances, the topical ophthalmological composition comprises about 0.05% (w/w) API. In some instances, the topical ophthalmological composition comprises about 1% (w/w) API. In some instances, the topical ophthalmological composition comprises about 5% to about 20% (w/w) MCT. In some instances, the topical ophthalmological composition is administered to the eye of the subject every about 6 to about 8 hours or three times a day. In some instances, the topical ophthalmological composition is administered to the subject once a day. In some instances, the topical ophthalmological composition is administered to the subject twice a day. In some instances, the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, or reducing one or more symptoms of the ocular condition. In some instances, the topical ophthalmological composition is non-aqueous. In some instances, the API is in a free base form. In some instances, the topical ophthalmological composition is absorbed transcorneally in the eye. In some instances, the topical ophthalmological composition is selectively absorbed transcorneally in the eye as compared to periorbitally.

In another aspect, described herein is a method for slowing myopia progression in a subject or for relieving vitreous floater symptoms in a subject, comprising administering to an eye of the subject a topical ophthalmological composition, wherein the topical ophthalmological composition comprises: about 0.001% to about 5% (w/w) API, wherein the API has a solubility in MCT of at least from about 0.01% to about 10% (w/w), and wherein the API is selected from atropine or an atropine mimetic; from about 1% to about 50% (w/w) of an MCT; and from about 50% to about 99% (w/w) of a semi-fluorinated alkane compound. In some instances, the topical ophthalmological composition comprises about 0.02% (w/w) API. In some instances, the topical ophthalmological composition comprises about 0.04% (w/w) API. In some instances, the topical ophthalmological composition comprises about 0.05% (w/w) API. In some instances, the topical ophthalmological composition comprises about 1% (w/w) API. In some instances, the topical ophthalmological composition comprises about 5% to about 20% (w/w) MCT. In some instances, the topical ophthalmological composition is administered to the eye of the subject every about 6 to about 8 hours or three times a day. In some instances, the topical ophthalmological composition is administered to the subject once a day. In some instances, the topical ophthalmological composition is administered to the subject twice a day. In some instances, the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for slowing myopia progression in a subject or for relieving vitreous floater symptoms in a subject. In some instances, the topical ophthalmological composition is non-aqueous. In some instances, the API is in a free base form. In some instances, the topical ophthalmological composition is absorbed transcorneally in the eye. In some instances, the topical ophthalmological composition is selectively absorbed transcorneally in the eye as compared to periorbitally.

In another aspect, described herein is a method for making the topical ophthalmological nano-emulsions described herein, comprising the steps of:
(a) combining an ocular API with MCT to form an MCT solution;
(b) combining said MCT solution with a semi-fluorinated alkane compound to form a mixture, wherein said mixture comprises from about 1% to about 50% said MCT solution and from about 50% to about 99% of said semi-fluorinated alkane compound; and
(c) emulsifying said mixture and reducing droplet particle size D90 within said mixture to less than 100 nm by physical agitation selected from high speed stirring, vortexing, sonicating, heating and stirring, or homogenizing the mixture until the droplet size reaches droplet particle size D90 of less than 100 nm.

In these and other embodiments, the method(s) can have the following optional features. The method can further comprise measuring the droplet particle size of the mixture during or following said physical agitation. Reducing droplet particle size D90 within said mixture to less than 100 nm can comprise reducing droplet particle size D90 within said mixture to less than 50 nm, less than 20 nm, less than 10 nm, less than 5 nm, or from about 2 to about 3 nm.

In some instances of the compositions, nano-emulsions, and methods herein, the API can be lifitegrast or a derivative, analogue, pharmaceutically acceptable salt, free base form, racemic mixture, or diastereomer or enantiomer thereof. In these and other instances, the compositions, nano-emulsions, and methods can have the following optional features. The MCT can be present in an amount of from about 1% to about 50% (w/w), optionally from about 1% to about 30% (w/w), from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a glyceryl tricaprylate/tricaprate and a glyceryl tricaprylate/tricaprylin. The lifitegrast or a derivative, analogue, pharmaceutically acceptable salt, free base form, racemic mixture, or diastereomer or enantiomer thereof can be present in the composition in an amount of from about 0.01 to about 10 mg/ml, optionally from about 0.01 to about 5 mg/ml, optionally from about 0.01 to about 1 mg/ml. The lifitegrast or a derivative, analogue, pharmaceutically acceptable salt, free base form, racemic mixture, or diastereomer or enantiomer thereof can be present in the composition in an amount of from about 0.05% to about 20% (w/w), optionally from about 0.05% to about 10% (w/w), optionally from about 0.05% to about 5% (w/w), optionally selected from about 0.05%, 0.1%, 0.5%, 1%, 2%, 3%, 4%, or 5% (w/w).

In some instances of the compositions, nano-emulsions, and methods herein, the API can be aceclidine, or a derivative, analogue, pharmaceutically acceptable salt, free base form, racemic mixture, or diastereomer or enantiomer thereof. In some instances of the methods described herein, the ocular condition can be presbyopia or myopia. In some instances, a method is provided for treating, slowing the progression of, improving one or more symptoms of, myopia or presbyopia in a subject, comprising administering to an eye of the subject a topical ophthalmological composition, wherein the topical ophthalmological composition comprises:
about 0.001% to about 10% (w/w) API, wherein the API is selected from aceclidine, or a derivative, analogue, pharmaceutically acceptable salt, free base form, racemic mixture, or diastereomer or enantiomer thereof,
from about 1% to about 50% (w/w) of an MCT; and
from about 50% to about 99% (w/w) of a semi-fluorinated alkane compound.

In these and other instances, the methods can have the following optional features. The topical ophthalmological composition can be administered to the eye of the subject every about 6 to about 8 hours or three times a day. The topical ophthalmological composition can be non-aqueous. The topical ophthalmological composition can be administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject.

The topical ophthalmological composition can be non-aqueous. The API can be in a free base form. The topical ophthalmological composition can be absorbed transcorneally in the eye. The topical ophthalmological composition can be selectively absorbed transcorneally in the eye as compared to periorbitally. In these and other instances, the compositions, nano-emulsions, and methods can have the following optional features. The MCT can be present in an amount of from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a glyceryl tricaprylate/tricaprate and a glyceryl tricaprylate/tricaprylin. The aceclidine, or a derivative, analogue, pharmaceutically acceptable salt, free base form, racemic mixture, or diastereomer or enantiomer thereof can be present in the composition in an amount of from about 1 to about 30 mg/ml, optionally from about 5 to about 20 mg/ml, optionally from about 10 to about 15 mg/ml. The aceclidine, or a derivative, analogue, pharmaceutically acceptable salt, free base form, racemic mixture, or diastereomer or enantiomer thereof can be present in the composition in an amount of from about 0.1% to about 10% (w/w), optionally from about 1% to about 3% (w/w), optionally about 1.75% (w/w). In some instances of the compositions, nano-emulsions, and methods herein, the API can be atropine, an atropine mimetic or analog, or a free base or salt form thereof. In these and other instances, the compositions, nano-emulsions, and methods can have the following optional features. The MCT can be present in an amount of from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a glyceryl tricaprylate/tricaprate and a glyceryl tricaprylate/tricaprylin. The atropine, atropine mimetic or analog, or a free base or salt form thereof can be present in the composition in an amount of from about 0.005 to about 1 mg/ml, optionally from about 0.01 to about 0.75 mg/ml, optionally from about 0.01 to about 0.1 mg/ml, optionally from about 0.05 to about 0.75 mg/ml, optionally from about 0.01 to about 0.03 mg/ml, optionally from about 0.1 to about 0.5 mg/ml. The atropine, atropine mimetic or analog, or a free base or salt form thereof can be present in the composition in an amount of from about 0.0005% to about 2% (w/w), optionally from about 0.001% to about 0.005%, 0.001% to about 0.01%, optionally from about 0.005% to about 0.5%, optionally from about 0.005% to about 0.1% (w/w), optionally from about 0.008% to about 0.02%, optionally about 0.01% (w/w).

In some instances of the compositions, nano-emulsions, and methods herein, the API can be AR-15512 ((1R,2S,5R)—N-(4-methoxyphenyl)-5-methyl-2-(1-methylethyl) cyclohexanecarboxamide) or a derivative, analogue, racemic mixture, or diastereomer or enantiomer thereof. In these and other instances, the compositions, nano-emulsions, and methods can have the following optional features. The MCT can be present in an amount of from about 0.1% to about 50% (w/w), optionally from about 0.1% to about 10% (w/w), from about 1% to about 30% (w/w), from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The AR-15512 ((1R,2S,5R)—N-(4-methoxyphenyl)-5-methyl-2-(1-methylethyl) cyclohexanecarboxamide) or a derivative, analogue, racemic mixture, or diastereomer or enantiomer thereof can be present in the composition in an amount of from about 0.001 mg/ml to 1 mg/ml, optionally 0.01 mg/ml to 1 mg/ml, 0.01 mg/ml to 0.1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, or about 0.1 mg/ml. The AR-15512 ((1R,2S,5R)—N-(4-methoxyphenyl)-5-methyl-2-(1-methylethyl) cyclohexanecarboxamide) or a derivative, analogue, racemic mixture, or diastereomer or enantiomer thereof can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).

In some instances of the compositions, nano-emulsions, and methods herein, the API can be AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof. In some instances of the methods described herein, the ocular condition can be presbyopia or myopia. In some instances, a method is provided for treating, slowing the progression of, improving one or more symptoms of, myopia or presbyopia in a subject, comprising administering to an eye of the subject a topical ophthalmological composition, wherein the topical ophthalmological composition comprises:
about 0.0001% to about 0.1% (w/w) API, wherein the API is selected from AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof,
from about 0.1% to about 50% (w/w) of an MCT; and
from about 50% to about 99% (w/w) of a semi-fluorinated alkane compound.

In these and other instances, the methods can have the following optional features. The topical ophthalmological composition can be administered to the eye of the subject every about 6 to about 8 hours or three times a day. The topical ophthalmological composition can be non-aqueous. The topical ophthalmological composition can be administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject. The topical ophthalmological composition can be non-aqueous. The API can be in a free base form. The topical ophthalmological composition can be absorbed transcorneally in the eye. The topical ophthalmological composition can be selectively absorbed transcorneally in the eye as compared to periorbitally. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof can be present in the composition in an amount of from about 1 to about 30 mg/ml, optionally from about 5 to about 20 mg/ml, optionally from about 10 to about 15 mg/ml. The AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).

In some instances of the compositions, nano-emulsions, and methods herein, the API can be AR-15512 ((1R,2S,5R)—N-(4-methoxyphenyl)-5-methyl-2-(1-methylethyl) cyclohexanecarboxamide), an AR-15512 mimetic or analog. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The AR-15512, AR-15512 mimetic or analog can be present in the composition in an amount of from about 0.005 to about 1 mg/ml, optionally from about 0.01 to about 0.75 mg/ml, optionally from about 0.01 to about 0.1 mg/ml, optionally from about 0.05 to about 0.75 mg/ml, optionally from about 0.01 to about 0.03 mg/ml, optionally from about 0.1 to about 0.5 mg/ml. The AR-15512, AR-15512 mimetic or analog can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).

In some embodiments, the compositions and methods provided herein can provide several advantages. First, in some instance, the compositions provided herein can provide increased solubility of the API. In some instances, the increased solubility the compositions provided herein can provide increased solubility of the API in non-aqueous formulations, for example increased solubility of the API in a SFA.

In some instances of the compositions, nano-emulsions, and methods herein, the API can be lotilaner, or a derivative, analogue, racemic mixture, or diastereomer or enantiomer thereof. In these and other instances, the compositions, nano-emulsions, and methods can have the following optional features. The MCT can be present in an amount of from about 0.1% to about 50% (w/w), optionally from about 0.1% to about 10% (w/w), from about 1% to about 30% (w/w), from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The lotilaner, or a derivative, analogue, racemic mixture, or diastereomer or enantiomer thereof can be present in the composition in an amount of from about 0.001 mg/ml to 10 mg/ml, optionally 0.01 mg/ml to 5 mg/ml, 0.01 mg/ml to 4 mg/ml, optionally 0.1 mg/ml to 5 mg/ml, optionally 1 mg/ml to 5 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 1 mg/ml, about 1.5 mg/ml, about 2 mg/ml, about 2.5 mg/ml, about 3 mg/ml, about 3.2 mg/ml, about 3.5 mg/ml, about 4 mg/ml, about 4.5 mg/ml, or about 5 mg/ml. The lotilaner, or a derivative, analogue, racemic mixture, or diastereomer or enantiomer thereof can be present in the composition in an amount of from about 0.0001% to 1% (w/w), optionally from about 0.001% to about 1% (w/w), optionally from about 0.0001% to 0.9%, optionally from about 0.0001% to 0.9%, optionally from about 0.0001% to 0.8%, optionally from about 0.0001% to 0.7%, optionally from about 0.0001% to 0.6%, optionally from about 0.0001% to 0.5%, optionally from about 0.0001% to 0.4%, optionally from about 0.0001% to 0.3%, optionally from about 0.001% to 0.9%, optionally from about 0.001% to 0.9%, optionally from about 0.001% to 0.8%, optionally from about 0.001% to 0.7%, optionally from about 0.001% to 0.6%, optionally from about 0.001% to 0.5%, optionally from about 0.001% to 0.4%, optionally from about 0.001% to 0.3%, optionally selected from about 0.01%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, or about 0.1% (w/w). In some instances, the lotilaner, or a derivative, analogue, racemic mixture, or diastereomer or enantiomer thereof can be present in the composition in an amount of about 0.25% (w/w).

In some instances of the compositions, nano-emulsions, and methods herein, the API can be lotilaner, or a derivative, analogue, racemic mixture, or diastereomer or enantiomer thereof. In some instances, a method is provided for aiding in eradication or reduction of parasite infestations of the eyes, ocular tissue, skin, eyelashes, or other body hair; for treating or reducing or relieving symptoms of certain diseases or conditions, such as ocular, skin, vector-borne, or other diseases cause by parasites; for treating or reducing or relieving symptoms of certain ocular diseases or conditions, such as blepharitis, meibomian gland disfunction, or rosacea caused by parasites such as Demodex mites; for treating or reducing or relieving symptoms of eyelid margin redness caused by Demodex mites, or improving eyelash health in a subject; for eradicating, reducing the population of, treating or reducing or relieving symptoms of lice, scabies, or bed bugs and associated skin, hair, scalp, or follicular conditions; for treating, slowing the progression of, improving one or more symptoms of, myopia or presbyopia in a subject, the method comprising administering to a subject in need thereof a topical ophthalmological composition, wherein the topical ophthalmological composition comprises:
about 0.0001% to about 1% (w/w) API, wherein the API is selected from lotilaner, or a derivative, analogue, racemic mixture, or diastereomer or enantiomer thereof,
from about 0.1% to about 50% (w/w) of an MCT; and
from about 50% to about 99% (w/w) of a semi-fluorinated alkane compound.
   In these and other instances, the methods can have the following optional features. The topical ophthalmological composition can be administered to the eye of the subject every about 6 to about 8 hours or three times a day. The topical ophthalmological composition can be non-aqueous. The topical ophthalmological composition can be administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject. The topical ophthalmological composition can be non-aqueous. The API can be in a free base form. The topical ophthalmological composition can be absorbed transcorneally in the eye. The topical ophthalmological composition can be selectively absorbed transcorneally in the eye as compared to periorbitally. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The lotilaner, or a derivative, analogue, racemic mixture, or diastereomer or enantiomer thereof can be present in the composition in an amount of from about 1 to about 30 mg/ml, optionally from about 5 to about 20 mg/ml, optionally from about 10 to about 15 mg/ml. In some instances, the lotilaner, or a derivative, analogue, racemic mixture, or diastereomer or an enantiomer thereof can be present in the composition in an amount of from 0.001 mg/ml to 10 mg/ml, optionally 0.01 mg/ml to 5 mg/ml, 0.01 mg/ml to 4 mg/ml, optionally 0.1 mg/ml to 5 mg/ml, optionally 1 mg/ml to 5 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 1 mg/ml, about 1.5 mg/ml, about 2 mg/ml, about 2.5 mg/ml, about 3 mg/ml, about 3.2 mg/ml, about 3.5 mg/ml, about 4 mg/ml, about 4.5 mg/ml, or about 5 mg/ml. In some instances, the lotilaner, or a derivative, analogue, racemic mixture, or diastereomer or an enantiomer thereof can be present in the composition in an amount of about 3.2 mg/ml. The lotilaner, or a derivative, analogue, racemic mixture, or diastereomer or enantiomer thereof can be present in the composition in an amount of from about 0.0001% to 1% (w/w), optionally from about 0.001% to about 1% (w/w), optionally from about 0.0001% to 0.9%, optionally from about 0.0001% to 0.9%, optionally from about 0.0001% to 0.8%, optionally from about 0.0001% to 0.7%, optionally from about 0.0001% to 0.6%, optionally from about 0.0001% to 0.5%, optionally from about 0.0001% to 0.4%, optionally from about 0.0001% to 0.3%, optionally from about 0.001% to 0.9%, optionally from about 0.001% to 0.9%, optionally from about 0.001% to 0.8%, optionally from about 0.001% to 0.7%, optionally from about 0.001% to 0.6%, optionally from about 0.001% to 0.5%, optionally from about 0.001% to 0.4%, optionally from about 0.001% to 0.3%, optionally selected from about 0.01%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, or about 0.1% (w/w). In some instances, the lotilaner, or a derivative, analogue, racemic mixture, or diastereomer or enantiomer thereof can be present in the composition in an amount of about 0.25% (w/w).

In some instances of the compositions, nano-emulsions, and methods herein, the API can be lotilaner, a lotilaner mimetic or analog. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The lotilaner, lotilaner mimetic or analog can be present in the composition in an amount of from 0.001 mg/ml to 10 mg/ml, optionally 0.01 mg/ml to 5 mg/ml, 0.01 mg/ml to 4 mg/ml, optionally 0.1 mg/ml to 5 mg/ml, optionally 1 mg/ml to 5 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 1 mg/ml, about 1.5 mg/ml, about 2 mg/ml, about 2.5 mg/ml, about 3 mg/ml, about 3.2 mg/ml, about 3.5 mg/ml, about 4 mg/ml, about 4.5 mg/ml, or about 5 mg/ml. In some instances, the lotilaner, or a derivative, analogue, racemic mixture, or diastereomer or an enantiomer thereof can be present in the composition in an amount of about 3.2 mg/ml. The lotilaner, or a derivative, analogue, racemic mixture, or diastereomer or enantiomer thereof can be present in the composition in an amount of from about 0.0001% to 1% (w/w), optionally from about 0.001% to about 1% (w/w), optionally from about 0.0001% to 0.9%, optionally from about 0.0001% to 0.9%, optionally from about 0.0001% to 0.8%, optionally from about 0.0001% to 0.7%, optionally from about 0.0001% to 0.6%, optionally from about 0.0001% to 0.5%, optionally from about 0.0001% to 0.4%, optionally from about 0.0001% to 0.3%, optionally from about 0.001% to 0.9%, optionally from about 0.001% to 0.9%, optionally from about 0.001% to 0.8%, optionally from about 0.001% to 0.7%, optionally from about 0.001% to 0.6%, optionally from about 0.001% to 0.5%, optionally from about 0.001% to 0.4%, optionally from about 0.001% to 0.3%, optionally selected from about 0.01%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, or about 0.1% (w/w). In some instances, the lotilaner, or a derivative, analogue, racemic mixture, or diastereomer or enantiomer thereof can be present in the composition in an amount of about 0.25% (w/w).

In some instances of the compositions, nano-emulsions, and methods herein, the API can be cyclosporine, or a derivative, analogue, pharmaceutically acceptable salt, free base form, racemic mixture, or diastereomer or enantiomer thereof. In these and other instances, the compositions, nano-emulsions, and methods can have the following optional features. The MCT can be present in an amount of from about 0.1% to about 50% (w/w), optionally from about 0.1% to about 10% (w/w), from about 1% to about 30% (w/w), from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The cyclosporine, or a derivative, analogue, pharmaceutically acceptable salt, free base form, racemic mixture, or diastereomer or enantiomer thereof can be present in the composition in an amount of from about 0.001 mg/ml to 10 mg/ml, optionally 0.01 mg/ml to 10 mg/ml, 0.01 mg/ml to 5 mg/ml, 0.01 mg/ml to 1 mg/ml, 0.1 mg/ml to 1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.65 mg/ml, about 0.7 mg/ml, about 0.8 mg/ml, about 0.9 mg/ml, about 1 mg/ml, about 5 mg/ml. In some instances, the cyclosporine, or a derivative, analogue, pharmaceutically acceptable salt, free base form, racemic mixture, or diastereomer or enantiomer thereof can be present in the composition in an amount of about 0.65 mg/ml. The cyclosporine, or a derivative, analogue, pharmaceutically acceptable salt, free base form, racemic mixture, or diastereomer or enantiomer thereof can be present in the composition in an amount of from about 0.0001% to 10% (w/w), optionally from about 0.01% to about 5%, optionally from about 0.01% to about 1%, optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.065%, about 0.07%, about 0.08%, about 0.08%, about 0.09%, or about 0.1% (w/w). In some instances, the cyclosporine, or a derivative, analogue, pharmaceutically acceptable salt, free base form, racemic mixture, or diastereomer or enantiomer thereof can be present in the composition in an amount of about 0.05% (w/w).

In some instances of the compositions, nano-emulsions, and methods herein, the API can be cyclosporine, or a derivative, analogue, pharmaceutically acceptable salt, free base form, racemic mixture, or diastereomer or enantiomer thereof. In some instances of the methods described herein, the ocular condition can be presbyopia or myopia. In some instances, a method is provided for treating, slowing the progression of, improving one or more symptoms of, dry eye syndrome or keratoconjunctivitis sicca, dry eye disease, inflammatory dry eye, redness, meibomian gland disfunction, myopia or presbyopia in a subject, comprising administering to an eye of the subject a topical ophthalmological composition, wherein the topical ophthalmological composition comprises:
about 0.0001% to about 10% (w/w) API, wherein the API is selected from cyclosporine, or a derivative, analogue, pharmaceutically acceptable salt, free base form, racemic mixture, or diastereomer or enantiomer thereof;
from about 0.1% to about 50% (w/w) of an MCT; and
from about 50% to about 99% (w/w) of a semi-fluorinated alkane compound.
   In these and other instances, the methods can have the following optional features. The topical ophthalmological composition can be administered to the eye of the subject every about 6 to about 8 hours or three times a day. The topical ophthalmological composition can be non-aqueous. The topical ophthalmological composition can be administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject. The topical ophthalmological composition can be non-aqueous. The API can be in a free base form. The topical ophthalmological composition can be absorbed transcorneally in the eye. The topical ophthalmological composition can be selectively absorbed transcorneally in the eye as compared to periorbitally. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The cyclosporine, or a derivative, analogue, pharmaceutically acceptable salt, free base form, racemic mixture, or diastereomer or enantiomer thereof can be present in the composition in an amount of from about 1 to about 30 mg/ml, optionally from about 5 to about 20 mg/ml, optionally from about 10 to about 15 mg/ml. The cyclosporine, or a derivative, analogue, pharmaceutically acceptable salt, free base form, racemic mixture, or diastereomer or enantiomer thereof can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w). In some instances, the cyclosporine, or a derivative, analogue, pharmaceutically acceptable salt, free base form, racemic mixture, or diastereomer or enantiomer thereof can be present in the composition in an amount of from about 0.001 mg/ml to 10 mg/ml, optionally 0.01 mg/ml to 10 mg/ml, 0.01 mg/ml to 5 mg/ml, 0.01 mg/ml to 1 mg/ml, 0.1 mg/ml to 1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.65 mg/ml, about 0.7 mg/ml, about 0.8 mg/ml, about 0.9 mg/ml, about 1 mg/ml, about 5 mg/ml. In some instances, the cyclosporine, or a derivative, analogue, pharmaceutically acceptable salt, free base form, racemic mixture, or diastereomer or enantiomer thereof can be present in the composition in an amount of about 0.65 mg/ml. In some instances, the cyclosporine, or a derivative, analogue, pharmaceutically acceptable salt, free base form, racemic mixture, or diastereomer or enantiomer thereof can be present in the composition in an amount of from about 0.0001% to 10% (w/w), optionally from about 0.01% to about 5%, optionally from about 0.01% to about 1%, optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.065%, about 0.07%, about 0.08%, about 0.08%, about 0.09%, or about 0.1% (w/w). In some instances, the cyclosporine, or a derivative, analogue, pharmaceutically acceptable salt, free base form, racemic mixture, or diastereomer or enantiomer thereof can be present in the composition in an amount of about 0.05% (w/w).

In some instances of the compositions, nano-emulsions, and methods herein, the API can be cyclosporine, a cyclosporine mimetic or analog. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The cyclosporine, cyclosporine mimetic or analog can be present in the composition in an amount of from about 0.005 to about 1 mg/ml, optionally from about 0.01 to about 0.75 mg/ml, optionally from about 0.01 to about 0.1 mg/ml, optionally from about 0.05 to about 0.75 mg/ml, optionally from about 0.01 to about 0.03 mg/ml, optionally from about 0.1 to about 0.5 mg/ml. The cyclosporine, cyclosporine mimetic or analog can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w). In some instances, the cyclosporine, or a derivative, analogue, pharmaceutically acceptable salt, free base form, racemic mixture, or diastereomer or enantiomer thereof can be present in the composition in an amount of from about 0.001 mg/ml to 10 mg/ml, optionally 0.01 mg/ml to 10 mg/ml, 0.01 mg/ml to 5 mg/ml, 0.01 mg/ml to 1 mg/ml, 0.1 mg/ml to 1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.65 mg/ml, about 0.7 mg/ml, about 0.8 mg/ml, about 0.9 mg/ml, about 1 mg/ml, about 5 mg/ml. In some instances, the cyclosporine, or a derivative, analogue, pharmaceutically acceptable salt, free base form, racemic mixture, or diastereomer or enantiomer thereof can be present in the composition in an amount of about 0.65 mg/ml. In some instances, the cyclosporine, or a derivative, analogue, pharmaceutically acceptable salt, free base form, racemic mixture, or diastereomer or enantiomer thereof can be present in the composition in an amount of from about 0.0001% to 10% (w/w), optionally from about 0.01% to about 5%, optionally from about 0.01% to about 1%, optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.065%, about 0.07%, about 0.08%, about 0.08%, about 0.09%, or about 0.1% (w/w). In some instances, the cyclosporine, or a derivative, analogue, pharmaceutically acceptable salt, free base form, racemic mixture, or diastereomer or enantiomer thereof can be present in the composition in an amount of about 0.05% (w/w).

In some instances of the compositions, nano-emulsions, and methods herein, the API can be ivermectin or a derivative, analogue, racemic mixture, or diastereomer or enantiomer thereof, optionally wherein the ivermectin is a mixture of ivermectin B1a and ivermectin B1b. In these and other instances, the compositions, nano-emulsions, and methods can have the following optional features. The MCT can be present in an amount of from about 0.1% to about 50% (w/w), optionally from about 0.1% to about 10% (w/w), from about 1% to about 30% (w/w), from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The ivermectin or a derivative, analogue, racemic mixture, or diastereomer or enantiomer thereof can be present in the composition in an amount of from about 0.001 mg/ml to 1 mg/ml, optionally 0.01 mg/ml to 1 mg/ml, 0.01 mg/ml to 0.1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, or about 0.1 mg/ml. In some instances, the ivermectin or a derivative, analogue, racemic mixture, or diastereomer or enantiomer thereof is present in the composition in an amount from 0.1 mg/ml to 50 mg/ml, optionally 0.1 mg/ml to 40 mg/ml, 0.1 mg/ml to 30 mg/ml, 0.1 mg/ml to 20 mg/ml, 0.1 mg/ml to 10 mg/ml, 1 mg/ml to 50 mg/ml, 10 mg/ml to 50 mg/ml, 20 mg/ml to 50 mg/ml, 30 mg/ml to 50 mg/ml, about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, or about 50 mg/ml. The ivermectin or a derivative, analogue, racemic mixture, or diastereomer or enantiomer thereof can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w). In some instances, the ivermectin or a derivative, analogue, racemic mixture, or diastereomer or enantiomer thereof is present in the composition in an amount from 0.01% to 5%, optionally 0.01% to 4%, 0.01% to 3%, 0.01% to 2%, 0.01% to 1%, 0.1% to 5%, 1% to 5%, 2% to 5%, 3% to 5%, about 0.1%, about 0.5%, about 0.1%, about 1.5%, about 2%, about 2.5%, about 3%, about 3.5%, about 4%, about 4.5%, or about 5%. In some instances, the ivermectin is a mixture of ivermectin B1a and ivermectin B1b

In some instances of the compositions, nano-emulsions, and methods herein, the API can be ivermectin or a derivative, analogue, racemic mixture, or diastereomer or enantiomer thereof, optionally wherein the ivermectin is a mixture of ivermectin B1a and ivermectin B1b. In some instances of the methods described herein, the ocular condition can be presbyopia or myopia. In some instances, a method is provided for treating, slowing the progression of, improving one or more symptoms of, demodex blepharitis, for aiding in eradication or reduction of parasite infestations of the eyes, ocular tissue, skin, eyelashes, or other body hair; for treating or reducing or relieving symptoms of certain diseases or conditions, such as ocular, skin, vector-borne, or other diseases cause by parasites; for treating or reducing or relieving symptoms of certain ocular diseases or conditions, such as blepharitis, meibomian gland disfunction, or rosacea caused by parasites such as Demodex mites; for treating or reducing or relieving symptoms of eyelid margin redness caused by Demodex mites, or improving eyelash health in a subject; for eradicating, reducing the population of, treating or reducing or relieving symptoms of lice, scabies, or bed bugs and associated skin, hair, scalp, or follicular conditions; for treating, slowing the progression of, improving one or more symptoms of, myopia or presbyopia in a subject, comprising administering to an eye of the subject a topical ophthalmological composition, wherein the topical ophthalmological composition comprises:
about 0.01% to about 5% (w/w) API, wherein the API is selected from ivermectin or a derivative, analogue, racemic mixture, or diastereomer or enantiomer thereof, optionally wherein the ivermectin is a mixture of ivermectin B1a and ivermectin B1b;
from about 0.1% to about 50% (w/w) of an MCT; and
from about 50% to about 99% (w/w) of a semi-fluorinated alkane compound.

In these and other instances, the methods can have the following optional features. The topical ophthalmological composition can be administered to the eye of the subject every about 6 to about 8 hours or three times a day. The topical ophthalmological composition can be non-aqueous. The topical ophthalmological composition can be administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject. The topical ophthalmological composition can be non-aqueous. The API can be in a free base form. The topical ophthalmological composition can be absorbed transcorneally in the eye. The topical ophthalmological composition can be selectively absorbed transcorneally in the eye as compared to periorbitally. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The axitinib, an axitinib mimetic or analog, or a free base or salt form thereof can be present in the composition in an amount of from about 1 to about 30 mg/ml, optionally from about 5 to about 20 mg/ml, optionally from about 10 to about 15 mg/ml. The ivermectin or a derivative, analogue, racemic mixture, or diastereomer or enantiomer thereof can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w). In some instances, the ivermectin or a derivative, analogue, racemic mixture, or diastereomer or enantiomer thereof is present in the composition in an amount from 0.1 mg/ml to 50 mg/ml, optionally 0.1 mg/ml to 40 mg/ml, 0.1 mg/ml to 30 mg/ml, 0.1 mg/ml to 20 mg/ml, 0.1 mg/ml to 10 mg/ml, 1 mg/ml to 50 mg/ml, 10 mg/ml to 50 mg/ml, 20 mg/ml to 50 mg/ml, 30 mg/ml to 50 mg/ml, about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, or about 50 mg/ml. In some instances, the ivermectin or a derivative, analogue, racemic mixture, or diastereomer or enantiomer thereof is present in the composition in an amount from 0.01% to 5%, optionally 0.01% to 4%, 0.01% to 3%, 0.01% to 2%, 0.01% to 1%, 0.1% to 5%, 1% to 5%, 2% to 5%, 3% to 5%, about 0.1%, about 0.5%, about 0.1%, about 1.5%, about 2%, about 2.5%, about 3%, about 3.5%, about 4%, about 4.5%, or about 5%. In some instances, the ivermectin is a mixture of ivermectin B1a and ivermectin B1b.

In some instances of the compositions, nano-emulsions, and methods herein, the API can be ivermectin, an ivermectin mimetic or analog. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The ivermectin, ivermectin mimetic or analog can be present in the composition in an amount of from about 0.005 to about 1 mg/ml, optionally from about 0.01 to about 0.75 mg/ml, optionally from about 0.01 to about 0.1 mg/ml, optionally from about 0.05 to about 0.75 mg/ml, optionally from about 0.01 to about 0.03 mg/ml, optionally from about 0.1 to about 0.5 mg/ml. The ivermectin, ivermectin mimetic or analog can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w). In some instances, the ivermectin or a derivative, analogue, racemic mixture, or diastereomer or enantiomer thereof is present in the composition in an amount from 0.1 mg/ml to 50 mg/ml, optionally 0.1 mg/ml to 40 mg/ml, 0.1 mg/ml to 30 mg/ml, 0.1 mg/ml to 20 mg/ml, 0.1 mg/ml to 10 mg/ml, 1 mg/ml to 50 mg/ml, 10 mg/ml to 50 mg/ml, 20 mg/ml to 50 mg/ml, 30 mg/ml to 50 mg/ml, about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, or about 50 mg/ml. In some instances, the ivermectin or a derivative, analogue, racemic mixture, or diastereomer or enantiomer thereof is present in the composition in an amount from 0.01% to 5%, optionally 0.01% to 4%, 0.01% to 3%, 0.01% to 2%, 0.01% to 1%, 0.1% to 5%, 1% to 5%, 2% to 5%, 3% to 5%, about 0.1%, about 0.5%, about 0.1%, about 1.5%, about 2%, about 2.5%, about 3%, about 3.5%, about 4%, about 4.5%, or about 5%.

In some instances of the compositions, nano-emulsions, and methods herein, the API can be axitinib, an axitinib mimetic or analog, or a free base or salt form thereof. In these and other instances, the compositions, nano-emulsions, and methods can have the following optional features. The MCT can be present in an amount of from about 0.1% to about 50% (w/w), optionally from about 0.1% to about 10% (w/w), from about 1% to about 30% (w/w), from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The axitinib, an axitinib mimetic or analog, or a free base or salt form thereof can be present in the composition in an amount of from about 0.001 mg/ml to 1 mg/ml, optionally 0.01 mg/ml to 1 mg/ml, 0.01 mg/ml to 0.1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, or about 0.1 mg/ml. The axitinib, an axitinib mimetic or analog, or a free base or salt form thereof can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).

In some instances of the compositions, nano-emulsions, and methods herein, the API can be axitinib, an axitinib mimetic or analog, or a free base or salt form thereof. In some instances of the methods described herein, the ocular condition can be presbyopia or myopia. In some instances, a method is provided for treating, slowing the progression of, improving one or more symptoms of, pinguecula, pterygium, an ocular condition associated with angiogenesis (e.g., hyperemia, neovascularization, pterygium, pinguecula, glaucoma filtration surgery and minimally invasive glaucoma surgery (MIGS), cornea transplant surgery with graft rejection, graft versus host disease, dry eye disease, atopic conjunctivitis, rosacea, ocular pemphigoid, Lyell's syndrome, Steven Johnson syndrome, viral infection (e.g. HSV-1), bacterial infection, fungal infection, parasitic infection, contact lens induced neovascularization, ulceration, alkali burns, and stem cell deficiency), myopia or presbyopia in a subject, comprising administering to an eye of the subject a topical ophthalmological composition, wherein the topical ophthalmological composition comprises:
about 0.0001% to about 0.1% (w/w) API, wherein the API is selected from axitinib, an axitinib mimetic or analog, or a free base or salt form thereof,
from about 0.1% to about 50% (w/w) of an MCT; and
from about 50% to about 99% (w/w) of a semi-fluorinated alkane compound.
   In these and other instances, the methods can have the following optional features. The topical ophthalmological composition can be administered to the eye of the subject every about 6 to about 8 hours or three times a day. The topical ophthalmological composition can be non-aqueous. The topical ophthalmological composition can be administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject. The topical ophthalmological composition can be non-aqueous. The API can be in a free base form. The topical ophthalmological composition can be absorbed transcorneally in the eye. The topical ophthalmological composition can be selectively absorbed transcorneally in the eye as compared to periorbitally. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The axitinib, an axitinib mimetic or analog, or a free base or salt form thereof can be present in the composition in an amount of from about 1 to about 30 mg/ml, optionally from about 5 to about 20 mg/ml, optionally from about 10 to about 15 mg/ml. In some instances, the axitinib, an axitinib mimetic or analog, or a free base or salt form thereof can be present in the composition in an amount of about 0.5 to about 1 mg/ml. The axitinib, an axitinib mimetic or analog, or a free base or salt form thereof can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w). In some instances, the axitinib, an axitinib mimetic or analog, or a free base or salt form thereof can be present in the composition in an amount of about 0.05 to about 0.1% (w/w).

In some instances of the compositions, nano-emulsions, and methods herein, the API can be axitinib, an axitinib mimetic or analog. In these and other instances, the compositions, nano-emulsions, and methods can have the following optional features. The MCT can be present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w). The semi-fluorinated alkane compound can be present in an amount of from about 70% to about 99% (w/w), optionally from about 80% to about 95% (w/w), optionally selected from about 80%, 85%, 90%, or 95% (w/w). The semi-fluorinated alkane compound can be selected from perfluorobutylheptane (F4H5) and perfluorohexyloctane (F6H8). The MCT can be selected from a coco-caprylate/caprate, a propylene glycol dicaprylocaprate, a glyceryl tricaprylate/tricaprate, and a glyceryl tricaprylate/tricaprylin. The axitinib, axitinib mimetic or analog can be present in the composition in an amount of from about 0.005 to about 1 mg/ml, optionally from about 0.01 to about 0.75 mg/ml, optionally from about 0.01 to about 0.1 mg/ml, optionally from about 0.05 to about 0.75 mg/ml, optionally from about 0.01 to about 0.03 mg/ml, optionally from about 0.1 to about 0.5 mg/ml. In some instances, the axitinib, axitinib mimetic or analog can be present in the composition in an amount of from about 0.5 to about 1 mg/ml. The axitinib, axitinib mimetic or analog can be present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w). In some instances, the axitinib, axitinib mimetic or analog can be present in the composition in an amount of from about 0.05% to 0.1% (w/w).

Second, in some instances of the compositions and methods provided herein, the compositions can provide better stability of the API as compared to, e.g., aqueous or other standard ocular formulations of the API. This provides several additional advantages, including, in some instances, the possibility to use certain APIs that were previously difficult to formulate for ocular use, or the prevention of formation of API-based impurities, or improved shelf life and storage time as compared to other standard ocular formulations or aqueous formulations of the API.

Third, in some instances, the compositions described herein can be made into nanoemulsions having droplet sizes below 100 nm, 50 nm, 15 nm, 10 nm, and even 5 nm. Without being bound by theory, it is believed the smaller droplet sizes can lead to improved longer term stability of the API in the formulation as compared to aqueous formulations, as well as increased availability of the API, improved tissue penetration and distribution of the API, and improved residence time in ocular tissues.

Fourth, in some instances, the dispenser provided herein can provide increased compatibility with the compositions described herein, e.g. compositions comprising MCT and/or SFA. The increased compatibility of the dispensers can lead to better stability of the compositions and of the API in the compositions, longer shelf life for the compositions, and improved delivery of the prescribed or desired amount of API to a patient's or subject's eye or ocular tissue.

Fifth, in some instances, the compositions (e.g., topical ophthalmological compositions or topical ophthalmological nano-emulsions) described herein can provide improved delivery of the API to the cornea, e.g., by transcorneal absorption, e.g., as compared to an aqueous composition comprising the API or an aqueous composition comprising a salt of the API. In some instances, the compositions described herein are selectively transcorneally absorbed (e.g., compared with periorbital absorption). The improved delivery of the API to the cornea can allow for lower doses to be administered to a subject, and/or more frequent lower doses. Thus, the improved delivery of the API to the cornea reduces one or more adverse side effects of an API and can be used to treat ocular conditions in subjects highly sensitive to the API.

Thus, in some instances, also described herein is a method of administering to a subject a topical ophthalmological composition for selective transcorneal absorption, the method comprising administering to an eye of the subject a composition (e.g., a topical ophthalmological composition or a topical ophthalmological nano-emulsion described here). In some instances, the topical ophthalmological composition is non-aqueous. In some instances, the API is in free base form. In some instances, the API is atropine.

In some instances, also described herein is a method of treating an ocular condition in a subject, the method comprising: (a) (i) selecting a subject in need of transcorneal absorption of an API; or (ii) selecting a subject having an ocular condition, wherein the ocular condition is in need of transcorneal absorption of an API; and (b) administering to an eye of the subject a composition described herein (e.g., a topical ophthalmological composition or a topical ophthalmological nano-emulsion). In some instances, the topical ophthalmological composition is non-aqueous. In some instances, the API is in free base form. In some instances, the API is atropine.

Subjects in need of transcorneal absorption of an API include subjects having high sensitivity to an API, subjects in need of a lower dose of an API, subjects having one or more adverse effects of an aqueous API (e.g., an adverse effect attributed to the API and not attributed to another component or feature of the composition comprising the API, e.g., pH or a preservative), subjects in need of treatment with an API having a target receptor in one or more of the cornea, the aqueous humor, or the iris, and subjects in need of treatment with an API that is associated with one or more adverse events in one or more of conjunctival, scleral, and ciliary body.

In some instances, also described herein is a method of selectively delivering an API to a cornea of an eye of a subject, the method comprising administering to the eye of the subject a composition described herein (e.g., a topical ophthalmological composition or a topical ophthalmological nano-emulsion). In some instances, the topical ophthalmological composition is non-aqueous. In some instances, the API is in free base form. In some instances, the API is atropine. In some instances, the composition is administered to the eye of the subject about every about 6 to about 8 hours or three times a day. In some instances, the composition is administered to the subject once a day. In some instances, the composition is administered to the subject twice a day. In some instances, the composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating an ocular condition or symptom thereof.

In some instances, also described herein is a method of treating an ocular condition in a subject, the method comprising administering to the subject a composition described herein (e.g., a topical ophthalmological composition or a topical ophthalmological nano-emulsion) comprising an API, wherein the composition is non-aqueous; and wherein the subject has failed to respond to treatment with an aqueous composition comprising the API, wherein the subject has had an adverse side effect from the API (or a salt thereof) after receiving an aqueous composition comprising the API (or a salt thereof), and/or wherein the subject is highly sensitive to an aqueous composition comprising the API. In some instances, the subject has had an adverse side effect from a salt of the API. In some instances, the subject has had an adverse side effect from the API in its free base form. In some instances, the API is in free base form. In some instances, the API is atropine. In some instances, the composition is administered to the eye of the subject about every about 6 to about 8 hours or three times a day. In some instances, the composition or the nano-emulsion is administered to the subject once a day. In some instances, the composition or the nano-emulsion is administered to the subject twice a day. In some instances, the topical ophthalmological composition or the topical ophthalmological nano-emulsion is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating the ocular condition.

In some instances, also described herein is method of formulating a topical ophthalmological composition for transcorneal absorption, the method comprising: (a) identifying an aqueous topical ophthalmological composition comprising an active pharmaceutical ingredient (API) for selective transcorneal absorption; (b) combining an API with medium chain triglyceride (MCT) and semi-fluorinated alkane compound to form a mixture; and (d) emulsifying said mixture; optionally wherein the topical ophthalmological composition for transcorneal absorption comprises about 0.001% to about 5% (w/w) API, from about 1% to about 50% (w/w) of the MCT, and from about 50% to about 99% (w/w) of the semi-fluorinated alkane compound, and optionally wherein the API has a solubility in medium chain triglyceride (MCT) of at least from about 0.01% to about 10% (w/w). In some instances, the topical ophthalmological composition comprises about 0.02% (w/w) API. In some instances, the topical ophthalmological composition comprises about 0.04% (w/w) API. In some instances, the topical ophthalmological composition comprises about 0.05% (w/w) API. In some instances, the topical ophthalmological composition comprises about 1% (w/w) API. In some instances, the topical ophthalmological composition comprises about 5% to about 20% (w/w) MCT. In some instances, the API is not atropine or a salt thereof. In some instances, the API is atropine. In some instances, the API is in a free base form. In some instances, the method further comprises, prior to step (a), identifying an aqueous topical ophthalmological composition comprising an API for selective transcorneal absorption. In some instances, the topical ophthalmological composition is non-aqueous.

This disclosure also relates to compositions and methods for treating ophthalmological conditions such as presbyopia. The compositions can comprise a muscarinic cholinergic receptor agonist and a semi-fluorinated alkane compound. In some instances the compositions can further comprise an organic cosolvent.

This disclosure is also based, at least in part, on a realization that certain compositions comprising semi-fluorinated alkane compounds can provide improved stability of muscarinic cholinergic receptor agonists for ophthalmological formulations. The formulations disclosed herein can retain efficacy and stability and can be used with minimal or no irritation in the eye of a subject.

Various compounds such as aceclidine, a muscarinic cholinergic receptor agonist, have been investigated for treatment of presbyopia, but these compounds can have problems with stability in traditional aqueous ophthalmological formulations. For example, aceclidine can break down rapidly in traditional aqueous formulations. Many attempts to improve stability of these compounds in pharmaceutical formulations can lead to eye irritation because of the compounds used to stabilize the active pharmaceutical ingredient. There remains a need for improved treatments for presbyopia, including compositions that retain stability and efficacy while also providing minimal or no irritation to the eye.

This disclosure also provides compositions comprising a muscarinic cholinergic receptor agonist and a semi-fluorinated alkane compound, as well as methods for treating presbyopia with the compositions disclosed herein. The compositions described herein provide improved stability of the muscarinic cholinergic receptor agonist without a negative effect on efficacy or eye irritation. In some instances, the formulations also provide the ability to use higher concentrations of the muscarinic cholinergic receptor agonist in the compositions.

In one aspect, described herein is a topical ophthalmological composition comprising: a therapeutically effective amount of a muscarinic cholinergic receptor agonist as an active pharmaceutical ingredient; and a semifluorinated alkane compound.

The composition can optionally include one or more of the following features. The semifluorinated alkane compound can be present as a liquid vehicle. The muscarinic cholinergic receptor agonist can be selected from the group consisting of aceclidine, pilocarpine, bethanechol, cevimeline, methacholine, xanomeline, and aprolidine. The semifluorinated alkane can be a compound of formula RFRH or of formula RFRHRF, wherein RF is a perfluorinated hydrocarbon with 15 or less carbon atoms, and wherein RH is a nonfluorinated hydrocarbon with 15 or less carbon atoms. The semifluorinated alkane can be selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyldecane (F6H10). The semifluorinated alkane can be F6H8 (perfluorohexyloctane). The semifluorinated alkane can be perfluorobutylheptane (F4H5).

The composition can optionally further comprise one or more organic cosolvents. The organic cosolvent can be selected from the group consisting of medium-chain triglycerides (MCT), liquid paraffin, vitamin E acetate, D-a-tocopherol, oleic acid, ethyl oleate and combinations thereof. The organic cosolvent can be medium-chain triglycerides (MCT) or medium-chain triglycerides (MCT) having two or three fatty acids each independently having an aliphatic tail of 6-12 carbon atoms.

In these and other embodiments, the composition can include the following optional features. The concentration of the organic cosolvent in the composition can be less than or equal to about 50% (w/w). The concentration of organic cosolvent in the composition can be from about 1% to 15% (w/w). The organic cosolvent can be present in a concentration of from about 1% (w/w) to about 50% (w/w) and the semi-fluorinated alkane compound is present in a concentration of from about 50% to about 99%. The muscarinic cholinergic receptor agonist can be present in a concentration of from about 0.1% to about 2.0% (w/w), 0.5% to about 2.0% (w/w), or 0.5% to about 1.5% (w/w). The muscarinic cholinergic receptor agonist can be aceclidine. The aceclidine can be in a free base form or a salt form. The aceclidine can be in free base form and the concentration of the aceclidine is from about 0.1% to about 2.0% (w/w), from about 0.5% to about 2.0% (w/w), or from about 0.5% to about 1.5% (w/w). The muscarinic cholinergic receptor agonist can be pilocarpine. The pilocarpine can be in a free base form or a salt form. The pilocarpine can be in free base form and the concentration of the pilocarpine is from about 0.1% to about 2.0% (w/w), from about 0.5% to about 2.0% (w/w), or from about 0.5% to about 1.5% (w/w). The topical ophthalmological composition can be a non-aqueous solution, a suspension, or an emulsion. The muscarinic cholinergic receptor agonist in the topical ophthalmological composition can be chemically stable at room temperature for at least 0.5 year, for at least 1 year, or for at least 2 years. The topical ophthalmological composition can cause minimal or no irritation in a mammalian eye.

In another aspect, provided herein are eye drops comprising any one of the topical ophthalmological compositions described herein.

In another aspect, provided herein are methods for treating presbyopia in a subject in need thereof, comprising administering any one of the topical ophthalmological compositions described herein. In such methods, the topical ophthalmological composition can optionally cause minimal or no irritation in the eye of the subject.

In another aspect, provided herein is a topical ophthalmological composition comprising: a therapeutically effective amount of aceclidine; and a semi-fluorinated alkane compound as liquid vehicle. The composition can optionally include one or more of the following features. The composition can further comprise medium-chain triglycerides (MCT) having two or three fatty acids each independently having an aliphatic tail of 6-12 carbon atoms. The aceclidine can be in free base form and can be present in a concentration of from about 0.1% to about 2.0% (w/w), from about 0.5% to about 2.0% (w/w), or from about 0.5% to about 1.5% (w/w).

In another aspect, provided herein is a topical ophthalmological composition comprising: a therapeutically effective amount of pilocarpine; and a semi-fluorinated alkane compound as liquid vehicle. The composition can optionally include one or more of the following features. The composition can further comprise medium-chain triglycerides (MCT) having two or three fatty acids each independently having an aliphatic tail of 6-12 carbon atoms. The pilocarpine can be in free base form and can be present in a concentration of from about 0.1% to about 2.0% (w/w), from about 0.5% to about 2.0% (w/w), or from about 0.5% to about 1.5% (w/w).

In another aspect, provided herein are methods for treating glaucoma in a subject in need thereof, comprising administering any one of the topical ophthalmological compositions described herein. In such methods, the topical ophthalmological composition can optionally cause minimal or no irritation in the eye of the subject.

In another aspect, provided herein are methods for improving the ability of a subject to focus on a near object, comprising administering any one of the topical ophthalmological compositions described herein. In such methods, the topical ophthalmological composition can optionally cause minimal or no irritation in the eye of the subject.

In another aspect, provided herein are methods for reducing intraocular pressure in an eye of a subject in need thereof, comprising administering any one of the topical ophthalmological compositions described herein. In such methods, the topical ophthalmological composition can optionally cause minimal or no irritation in the eye of the subject.

In some embodiments, the compositions and methods provided herein can provide several advantages. This disclosure is based on the discovery that muscarinic cholinergic receptor agonists, such as, e.g., aceclidine, can be dissolved, or form a stable emulsion in a semifluorinated alkane compound at sufficient concentration to have biological efficacy. The formulation of muscarinic cholinergic receptor agonists, such as aceclidine, in a semifluorinated alkane compound, such as F6H8, can be stable for prolonged times at room temperature. Such formulations can be made into useful products with sufficient shelf-life for medical use and regulatory approval. These formulations can be non-irritating in the eye when dosed at a concentration higher than what is needed for some indications.

In some embodiments, the compositions described herein can exhibit greater stability of the muscarinic cholinergic receptor agonist than, e.g., aqueous compositions of the same compound. In some embodiments, this improved stability can allow the use of compositions that are preservative free or free of additional excipients or other irritants used in aqueous formulations to confer stability, thus reducing the possibility of irritation, such as eye irritation, or allergic reactions related to the inclusion of such additives.

In some embodiments, the compositions described herein can exhibit improved pharmacokinetics profiles to modulate certain target tissues, such as, e.g., pupil tissue, for more effective treatments of certain conditions, such as, e.g., presbyopia.

Other features and advantages of the present application will be apparent from the following detailed description and figures, and from the claims.

### BRIEF DESCRIPTION OF THE DRAWINGS

FIG. 1 is a graph showing efficacy of an exemplary embodiment of a nano-emulsion according to Example 4.

FIG. 2 is a graph showing pupil diameter change in rabbits after administration of an exemplary embodiment of a nano-emulsion according to Example 4.

FIG. 3 is a graph showing pupil diameter in humans after administration of an exemplary embodiment of a nano-emulsion according to Example 4.

FIG. 4 is an exploded, schematic cross-sectional view of an exemplary embodiment of a dispenser as described herein.

FIG. 5 is a series of graphs depicting drug concentrations in ocular tissues at different times after topical ocular dosing of atropine-10% MCT-F6H8 to both eyes in rabbits (n=2).

FIG. 6 is series of graphs depicting drug residue in ocular tissues at different time after topical ocular dosing of atropine-10% MCT-F6H8 to both eyes in rabbits (n=4 eyes). Top (A): bars for each timepoint, from left to right, are: cornea, conjunctiva, sclera, aqueous humor, iris-ciliary, retina-choroid, and vitreous humor. Middle (B): bars for each timepoint, from left to right, are: cornea and conjunctiva. Bottom (C): bars for each timepoint, from left to right, are: sclera, aqueous humor, iris-ciliary, retina-choroid, and vitreous humor.

FIG. 7 is a GC chromatogram of an exemplary formulation according to Example 17.

FIG. 8A is a graph showing the effects of an exemplary formulation on pupil diameter, according to Example 21. *P<0.05 or **P<0.01 compared with baseline using the T-TEST.

FIG. 8B is a graph showing changes in pupil diameter with an exemplary formulation, according to Example 21. *P<0.05 or **P<0.01 compared with baseline using the T-TEST.

FIG. 9A is a graph showing effects on pupil diameter of three exemplary formulations according to Example 22.

FIG. 9B is a graph showing changes in pupil diameter of three exemplary formulations according to Example 22.

### DETAILED DESCRIPTION

Provided herein are compositions for topical delivery of ophthalmological active pharmaceutical ingredients (APIs), comprising medium chain triglycerides (MCT), a semi-fluorinated alkane (SFA), and an active pharmaceutical ingredient (API), and methods for making and using the compositions, such as methods for treating or slowing the progression of, or reducing one or more symptoms of, an ocular condition in a subject. Also provided herein are dispensers or containers comprising the compositions.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the term “about” means “approximately” (e.g., plus or minus approximately 10% of the indicated value).

As used herein, the term “contacting” refers to the bringing together of indicated moieties in an in vitro system or an in vivo system. For example, “contacting” the eye tissue with a compound of the invention includes the administration of a compound of the present invention to an individual or patient, such as a human, having the eye tissue in need of treatment, as well as, for example, introducing a compound of the invention into a sample containing a cellular or purified preparation containing the ocular tissue.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans. In some embodiment, a human subject may be of any age, for example, from 0 to 100 years old. As used herein, “pediatric subject” and “child” are used interchangeably and refer to subjects from 0 to 18 years old, for example, 1, 2, 5, 10, 12, or 14 years old.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

In some instances, provided herein are compositions comprising an active pharmaceutical ingredient (API), wherein the API is soluble in MCT, a medium chain triglyceride (MCT), and a semi-fluorinated alkane compound.

In some instances of the compositions described herein, the composition is non-aqueous. In some instances of the compositions described herein, the composition is an oil-in-oil emulsion.

The compositions described herein can include an active pharmaceutical ingredient (API) compound. APIs, their analogs, their salts, and their free base forms (where applicable) are included within the scope of the present invention. Such solvates include for example hydrates, alcoholates (e.g., ethanol solvate), and the like. APIs can exist in different polymorphic forms. Such forms are intended to be included within the scope of the present disclosure.

Exemplary, non-limiting APIs useful in the compositions and nano-emulsions described herein can include alpha agonists, antibiotics, anti-parasitics, pressure regulators, topical transient receptor potential melastatin 8 (TRPM8) agonists, inhibitors of inflammatory cell binding, corticosteroids, anticholinergic agents (e.g., antimuscarinics and antinicotinics), antiglaucoma agents (e.g., alpha agonists, beta-blockers, carbonic anhydrase inhibitors, miotics, prostaglandin agonists, and the like), antihistamines, antivirals, cycloplegics/mydriatics, mast cell stabilizers, miotics (e.g., cholinesterase inhibitors, direct-acting miotics, and the like), ophthalmic non-steroidal anti-inflammatory drugs (NSAIDs), cyclosporine, riboflavin 5′-phosphate ophthalmic compounds, and vascular endothelial growth factor (VEGF) and VEGF receptor inhibitors.

Exemplary, nonlimiting antibiotics can include Moxifloxacin, Levofloxacin, Ofloxacin, Besifloxacin, Gatifloxacin, Azithromycin, Tobramycin, Erythromycin, Bacitracin, Neomycin-Polymyxin B-Gramicidin, Neomycin-Polymyxin B-Bacitracin, Bacitracin-Polymyxin B, and Sulfacetamide.

Exemplary, nonlimiting anti-parasitics can include formamidine parasiticides or isooxazoline parasiticides, such as but not limited to fluralaner, sarolaner, lotilaner, afoxolaner, and/or fluxametamide.

An additional exemplary, nonlimiting anti-parasitic is ivermectin (e.g., ivermectin B1a, ivermectin B1b, or a combination thereof). In some instances, the anti-parasitic is ivermectin (e.g., ivermectin B1a, ivermectin B1b, or a combination thereof), or a derivative, analogue, and L- and D-isomer thereof, including but not limited to enantiomers, compositions comprising racemic mixtures, and enantiomerically pure compositions. In some instances, the anti-parasitic comprises a mixture of ivermectin B1 homologues (e.g., a mixture of ivermectin B1a and ivermectin B1b). In some instances, the anti-parasitic comprises a mixture of ivermectin B1a and ivermectin B1b. Ivermectin B1a has the structure of Compound (I):

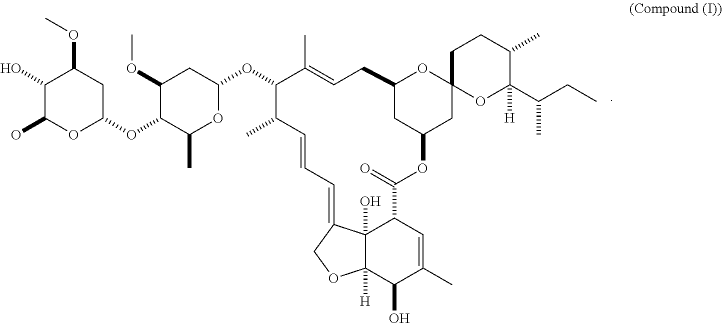

Ivermectin B1b has the structure of Compound (II):

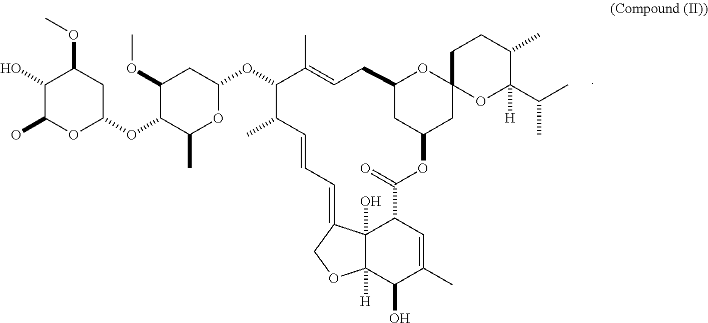

In some instances, the anti-parasitic comprises a mixture of ivermectin B1a and ivermectin B1b at a ivermectin B1a:ivermectin B1b ratio of about 1000:1, about 500:1, about 400:1, about 300:1, about 200:1, about 100:1, about 90:1, about 80:1, about 70:1, about 60:1, about 50:1, about 40:1, about 30:1, about 20:1, about 10:1, about 5:1, about 4:1, about 2:1, or about 1:1. In some instances, the anti-parasitic comprises a mixture of ivermectin B1a and ivermectin B1b, wherein the amount of ivermectin B1a in the anti-parasitic is about 1-, about 1.25-, about 1.5-, about 2-, about 3-, about 4-, about 5-, about 6-, about 7-, about 10-, about 11-, about 12-, about 13-, about 14-, about 15-, about 20-, about 25-, about 30-, about 35-, about 40-, about 45-, about 50-, about 55-, about 60-, about 70-, about 80-, about 90-, about 100-, about 200-, about 300-, about 400-, about 500-, about 750-, or about 1000-fold higher than the amount of ivermectin B1b in the anti-parasitic.

Exemplary, nonlimiting steroids and NSAIDs can include Fluorometholone, Loteprednol, Rimexolone, Prednisolone, and Difluprednate.

Exemplary, nonlimiting antimicrobials can include Natamycin, Ganciclovir, Trifluiridine, and Cysteamine.

Exemplary, nonlimiting pressure regulators can include Brimonidine, Apraclonidine Iopidine, Dorzolamide, Brinzolamide, Azopt, Timolol, Timoptic/Istalol/Betimol, Betaxolol, Betoptic, Levobunolol, Betagan, Metipranolol, Optipranolol, Latanoprost, Xalatan Green, Bimatoprost, Lumigan, Tafluprost, Zioptan, Latanoprostene, Vyzulta Turquoise, Pilocarpine, Carbachol, Isopto Carbachol, Netarsudil, and Rhopressa.

Exemplary, nonlimiting dilation and cycloplegia agents include Atropine, Isopto Atropine, Atropisol, Scopolamine, Isopto Hyoscine Red, Cyclopentolate, Cyclogyl, AK Pentolate, Tropicamide, Mydriacyl, Tropicacyl, and Phenylephrine.

In some instances, the API can be a muscarinic receptor agonist. Exemplary non-limiting muscarinic receptor agonists can include acetylcholine, arecoline, oxotremorine, muscarine, carbachol, nebracetam, bethanechol, pilocarpine, and desmethylclozapine, or a pharmaceutically acceptable salt thereof. Suitable examples of muscarinic receptor antagonists include atropine, hyoscyamine, scopolamine, diphenhydramine, dimenhydrinate, dicycloverine, tolterodine, oxybutynin, ipratropium, pirenzepine, telenzepine, tripitramine, gallamine, methoctramine, darifenacin, tiotropium, chlorpromazine, and haloperidol, a free base form, or a pharmaceutically acceptable salt thereof.

In some instances, the API can be a muscarinic receptor antagonist. Exemplary non-limiting muscarinic receptor antagonists can include atropine, hyoscyamine, scopolamine, diphenhydramine, dimenhydrinate, dicycloverine, tolterodine, oxybutynin, ipratropium, pirenzepine, telenzepine, tripitramine, gallamine, methoctramine, darifenacin, tiotropium, chlorpromazine, and haloperidol, a free base form, or a pharmaceutically acceptable salt thereof.

In some instances, the API is not a muscarinic receptor antagonist. In some instances, the API is not atropine or a salt thereof.

In some instances, the API is in a free base form.

In some instances, the API is in a free acid form.

In some instances, the API can be present in the compositions or nano-emulsions described herein in an amount of from about 0.001% to about 5% (w/w), from about 0.01% to about 5% (w/w), from about 0.1% to about 5% (w/w), from about 0.2% to about 50% (w/w), from about 0.50% to about 50% (w/w), from about 10% to about 50% (w/w), from about 2% to about 5% (w/w), from about 3% to about 5% (w/w), from about 4% to about 5% (w/w), from about 0.001% to about 2% (w/w), from about 0.001% to about 1% (w/w), from about 0.001% to about 0.9% (w/w), from about 0.001% to about 0.8% (w/w), from about 0.001% to about 0.7% (w/w), from about 0.001% to about 0.6% (w/w), from about 0.001% to about 0.5% (w/w), from about 0.001% to about 0.4% (w/w), from about 0.001% to about 0.3% (w/w), from about 0.001% to about 0.2% (w/w), from about 0.001% to about 0.1% (w/w), from about 0.01% to about 2% (w/w), from about 0.010% to about 10% (w/w), from about 0.010% to about 0.9% (w/w), from about 0.01% to about 0.8% (w/w), from about 0.01% to about 0.7% (w/w), from about 0.01% to about 0.6% (w/w), from about 0.01% to about 0.5% (w/w), from about 0.01% to about 0.4% (w/w), from about 0.01% to about 0.3% (w/w), from about 0.01% to about 0.2% (w/w), from about 0.01% to about 0.1% (w/w), from about 0.001% to about 2% (w/w), from about 0.001% to about 1% (w/w), from about 0.1% to about 0.9% (w/w), from about 0.1% to about 0.8% (w/w), from about 0.1% to about 0.7% (w/w), from about 0.1% to about 0.6% (w/w), from about 0.1% to about 0.5% (w/w), from about 0.1% to about 0.4% (w/w), from about 0.1% to about 0.3% (w/w), and from about 0.1% to about 0.2% (w/w). In some instances, the API can be present in the compositions or nano-emulsions described herein in an amount of about 0.02% (w/w). In some instances, the API can be present in the compositions or nano-emulsions described herein in an amount of about 0.04% (w/w). In some instances, the API can be present in the compositions or nano-emulsions described herein in an amount of about 0.05% (w/w). In some instances, the API can be present in the compositions or nano-emulsions described herein in an amount of about 1% (w/w).

In some instances of the compositions or nano-emulsions described herein, the API in the topical ophthalmological composition is chemically stable for at least 6 months, at least 1 year, at least 18 months, or at least 2 years.

In some instances, provided herein are compositions comprising a therapeutically effective amount of anti-parasitic compound as an active pharmaceutical ingredient; a semi-fluorinated alkyl alcohol; and a semi-fluorinated alkane compound. The compositions can, in some instances, be topical ophthalmological compositions. Anti-parasitic compounds can, in some instances, aid in eradication or reduction of parasite infestations of the eyes, ocular tissue, skin, eyelashes, or other body hair. In some instances, this can temporarily or permanently treat or reduce or relieve symptoms of certain diseases or conditions, such as ocular, skin, vector-borne, or other diseases cause by parasites. In some instances, anti-parasitic compounds can temporarily or permanently treat or reduce or relieve symptoms of certain ocular diseases or conditions, such as blepharitis, meibomian gland disfunction, or rosacea caused by parasites such as Demodex mites. In some instances, anti-parasitic compounds can temporarily or permanently treat or reduce or relieve symptoms of eyelid margin redness caused by Demodex mites, or improve eyelash health in a subject. In some instances, anti-parasitic compounds can temporarily or permanently eradicate, reduce the population of, treat or reduce or relieve symptoms of lice, scabies, or bed bugs and associated skin, hair, scalp, or follicular conditions. In some instances, the anti-parasitic compound can be an isooxazoline parasiticide or a formamidine parasiticide. In some instances, the anti-parasitic compound can be an isooxazoline parasiticide selected from fluralaner, sarolaner, lotilaner, afoxolaner, and/or fluxametamide, including derivatives, analogues, and L- and D-isomers thereof, including but not limited to enantiomers, compositions comprising racemic mixtures, and enantiomerically pure compositions. In some instances, the anti-parasitic compound can be ivermectin (e.g., ivermectin B1a, ivermectin B1b, or a combination thereof), or a derivative, analogue, isomer thereof, including but not limited to enantiomers, compositions comprising racemic mixtures, and enantiomerically pure compositions. In some instances, the anti-parasitic compound comprises a mixture of ivermectin B1 homologues (e.g., a mixture of ivermectin B1a and ivermectin B1b). In some instances, the anti-parasitic compound can be in free base form.

In some instances, the anti-parasitic compound can be present in an amount of from about 0.1% to about 5% (w/w), from about 0.1% to about 2.5% (w/w), from about 0.1% to about 2% (w/w), from about 0.1% to about 1.5% (w/w), from about 0.1% to about 1% (w/w), from about 0.1% to about 0.5% (w/w), from about 0.1% to about 0.45% (w/w), from about 0.1% to about 0.4% (w/w), from about 0.1% to about 0.35% (w/w), from about 0.1% to about 0.3% (w/w), from about 0.1% to about 0.25% (w/w), from about 0.1% to about 0.2% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.50% (w/w), from about 0.50% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), or about 2% (w/w).

In some instances, the anti-parasitic compound can be lotilaner or TP-03. In some instances, the lotilaner or TP-03 can be in a form including derivatives, analogues, and L- and D-isomers thereof, including but not limited to enantiomers, compositions comprising racemic mixtures, and enantiomerically pure compositions. In some instances, the lotilaner or TP-03 can be in free base form. In some instances, the lotilaner or TP-03 can be present in an amount of from about 0.1% to about 5% (w/w), from about 0.1% to about 2.5% (w/w), from about 0.1% to about 2% (w/w), from about 0.1% to about 1.5% (w/w), from about 0.1% to about 1% (w/w), from about 0.1% to about 0.5% (w/w), from about 0.10% to about 0.45% (w/w), from about 0.10% to about 0.4% (w/w), from about 0.10% to about 0.35% (w/w), from about 0.10% to about 0.3% (w/w), from about 0.1% to about 0.25% (w/w), from about 0.1% to about 0.2% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.50% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), or about 2% (w/w). In some instances, the lotilaner, or a derivative, analogue, racemic mixture, or diastereomer or enantiomer thereof can be present in the composition in an amount of about 0.25% (w/w).

In some instances, the anti-parasitic compound can be ivermectin (e.g., ivermectin B1a, ivermectin B1b, or a combination thereof). In some instances, the ivermectin can be in a form including derivatives, analogues, and L- and D-isomers thereof, including but not limited to enantiomers, compositions comprising racemic mixtures, and enantiomerically pure compositions. In some instances, the ivermectin can be a mixture of ivermectin B1a and ivermectin B1b. In some instances, the ivermectin (e.g., a mixture of ivermectin B1a and ivermectin B1b) can be present in an amount of from about 0.1% to about 5% (w/w), from about 0.1% to about 2.5% (w/w), from about 0.1% to about 2% (w/w), from about 0.1% to about 1.5% (w/w), from about 0.1% to about 1% (w/w), from about 0.1% to about 0.5% (w/w), from about 0.1% to about 0.45% (w/w), from about 0.10% to about 0.4% (w/w), from about 0.10% to about 0.35% (w/w), from about 0.10% to about 0.3% (w/w), from about 0.1% to about 0.25% (w/w), from about 0.1% to about 0.2% (w/w), from about 0.15% to about 0.5% (w/w), from about 0.15% to about 0.45% (w/w), from about 0.150% to about 0.4% (w/w), from about 0.150% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), or about 2% (w/w). In some instances, the anti-parasitic compound can be ivermectin (e.g., ivermectin B1a, ivermectin B1b, or a combination thereof), or a derivative, analogue, and L- and D-isomer thereof, including but not limited to enantiomers, compositions comprising racemic mixtures, and enantiomerically pure compositions. In some instances, the anti-parasitic compound comprises a mixture of ivermectin B1 homologues (e.g., a mixture of ivermectin B1a and ivermectin B1b). In some instances, the anti-parasitic compound comprises a mixture of ivermectin B1a and ivermectin B1b. In some instances, the anti-parasitic comprises a mixture of ivermectin B1a and ivermectin B1b at a ivermectin B1a:ivermectin B1b ratio of about 1000:1, about 500:1, about 400:1, about 300:1, about 200:1, about 100:1, about 90:1, about 80:1, about 70:1, about 60:1, about 50:1, about 40:1, about 30:1, about 20:1, about 10:1, about 5:1, about 4:1, about 2:1, or about 1:1. In some instances, the anti-parasitic comprises a mixture of ivermectin B1a and ivermectin B1b, wherein the amount of ivermectin B1a in the anti-parasitic is about 1-, about 1.25-, about 1.5-, about 2-, about 3-, about 4-, about 5-, about 6-, about 7-, about 10-, about 11-, about 12-, about 13-, about 14-, about 15-, about 20-, about 25-, about 30-, about 35-, about 40-, about 45-, about 50-, about 55-, about 60-, about 70-, about 80-, about 90-, about 100-, about 200-, about 300-, about 400-, about 500-, about 750-, or about 1000-fold higher than the amount of ivermectin B1b in the anti-parasitic. In some instances, the ivermectin or a derivative, analogue, racemic mixture, or diastereomer or enantiomer thereof is present in the composition in an amount from 0.1 mg/ml to 50 mg/ml, optionally 0.1 mg/ml to 40 mg/ml, 0.1 mg/ml to 30 mg/ml, 0.1 mg/ml to 20 mg/ml, 0.1 mg/ml to 10 mg/ml, 1 mg/ml to 50 mg/ml, 10 mg/ml to 50 mg/ml, 20 mg/ml to 50 mg/ml, 30 mg/ml to 50 mg/ml, about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, or about 50 mg/ml. In some instances, the ivermectin or a derivative, analogue, racemic mixture, or diastereomer or enantiomer thereof is present in the composition in an amount from 0.01% to 5%, optionally 0.01% to 4%, 0.01% to 3%, 0.01% to 2%, 0.01% to 1%, 0.1% to 5%, 1% to 5%, 2% to 5%, 3% to 5%, about 0.1%, about 0.5%, about 0.1%, about 1.5%, about 2%, about 2.5%, about 3%, about 3.5%, about 4%, about 4.5%, or about 5%.

In some instances, provided herein are compositions comprising a therapeutically effective amount of lifitegrast or a derivative, analogue, pharmaceutically acceptable salt, free base form, racemic mixture, or diastereomer or enantiomer thereof as an active pharmaceutical ingredient; a semi-fluorinated alkyl alcohol; and a semifluorinated alkane compound. The compositions can, in some instances, be topical ophthalmological compositions.

Lifitegrast is an N-acyl-L-alpha-amino acid obtained by formal condensation of the carboxy group of N-[2-(1-benzofuran-6-carbonyl)]-5,7-dichloro-1,2,3,4-tetrahydroisoquinoline-6-carboxylic acid with the amino group of 3-(methanesulfonyl)-L-phenylalanine. Lifitegrast can reduce inflammation by inhibiting inflammatory cell binding through inhibition of an integrin, lymphocyte function-associated antigen 1 (LFA-1), from binding to intercellular adhesion molecule 1 (ICAM-1). Lifitegrast, or a derivative, analogue, pharmaceutically acceptable salt, free base form, racemic mixture, or diastereomer or enantiomer thereof, can, in some instances, aid in treatment of dry eye syndrome, or keratoconjunctivitis sicca. In some instances, lifitegrast or a derivative, analogue, pharmaceutically acceptable salt, free base form, racemic mixture, or diastereomer or enantiomer thereof, can slow the progression of, treat, or reduce or relieve symptoms of certain ocular diseases or conditions, such as dry eye syndrome or keratoconjunctivitis sicca, dry eye disease, inflammatory dry eye, redness, or meibomian gland disfunction.

In some instances, the lifitegrast or a derivative, analogue, pharmaceutically acceptable salt, free base form, racemic mixture, or diastereomer or enantiomer thereof, can be present in an amount of from about 0.1% to about 10% (w/w), from about 0.1% to about 9% (w/w), from about 0.1% to about 8% (w/w), from about 0.1% to about 7% (w/w), from about 0.1% to about 6% (w/w), from about 0.1% to about 5% (w/w), from about 0.2% to about 10% (w/w), from about 0.3% to about 10% (w/w), from about 0.4% to about 10% (w/w), from about 0.5% to about 10% (w/w), from about 0.6% to about 10% (w/w), from about 0.7% to about 10% (w/w), from about 0.8% to about 10% (w/w), from about 0.9% to about 10% (w/w), from about 1% to about 10% (w/w), from about 1.5% to about 10% (w/w), from about 2% to about 10% (w/w), from about 2.5% to about 10% (w/w), from about 3% to about 10% (w/w), from about 3.5% to about 10% (w/w), from about 4% to about 10% (w/w), from about 4.5% to about 10% (w/w), from about 5% to about 10% (w/w), from about 5.5% to about 10% (w/w), from about 6% to about 10% (w/w), from about 6.5% to about 10% (w/w), from about 7% to about 10% (w/w), from about 7.5% to about 10% (w/w), from about 8% to about 10% (w/w), from about 8.5% to about 10% (w/w), from about 9% to about 10% (w/w), from about 9.5% to about 10% (w/w), from about 1% to about 5% (w/w), from about 2% to about 8% (w/w), from about 3% to about 9% (w/w), from about 4% to about 9% (w/w), from about 0.1% to about 2.5% (w/w), from about 0.1% to about 2% (w/w), from about 0.1% to about 1.5% (w/w), from about 0.1% to about 1% (w/w), from about 0.1% to about 0.5% (w/w), from about 0.1% to about 0.45% (w/w), from about 0.1% to about 0.4% (w/w), from about 0.10% to about 0.35% (w/w), from about 0.10% to about 0.3% (w/w), from about 0.10% to about 0.25% (w/w), from about 0.1% to about 0.2% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.30% to about 1.30% (w/w), from about 0.30% to about 10% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.10% (w/w), about 0.2% (w/w), about 0.30% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w), about 2.5% (w/w), about 3% (w/w), about 3.5% (w/w), about 4% (w/w), about 4.5% (w/w), about 5% (w/w), or about 5.5% (w/w). In some instances, the lifitegrast or a derivative, analogue, pharmaceutically acceptable salt, free base form, racemic mixture, or diastereomer or enantiomer thereof, can be present in an amount of from about 0.1% to about 10% (w/w).

In some instances, provided herein are compositions comprising a therapeutically effective amount of AR-15512 ((1R,2S,5R)—N-(4-methoxyphenyl)-5-methyl-2-(1-methylethyl) cyclohexanecarboxamide), or a derivative, analogue, pharmaceutically acceptable salt, free base form, racemic mixture, or diastereomer or enantiomer thereof as an active pharmaceutical ingredient; a semi-fluorinated alkyl alcohol; and a semi-fluorinated alkane compound. The compositions can, in some instances, be topical ophthalmological compositions.

AR-15512 can act as a potent and selective agonist of the TRPM8 (transient receptor potential melastatin 8) cold thermoreceptor. TRPM8 is a calcium-permeable ion channel, serves as a detector of cold in humans, and is involved in the regulation of blink rate and tear production. Stimulation of this receptor can lead to restoration of tear film volume and a reduction of ocular discomfort in patients suffering from dry eye symptoms or dry eye syndrome.

AR-15512, or a derivative, analogue, pharmaceutically acceptable salt, free base form, racemic mixture, or diastereomer or enantiomer thereof, can, in some instances, aid in treatment of dry eye syndrome, or keratoconjunctivitis sicca. In some instances, AR-15512 or a derivative, analogue, pharmaceutically acceptable salt, free base form, racemic mixture, or diastereomer or enantiomer thereof, can slow the progression of, treat, or reduce or relieve symptoms of certain ocular diseases or conditions, such as dry eye syndrome or keratoconjunctivitis sicca, dry eye disease, inflammatory dry eye, redness, or meibomian gland disfunction.

In some instances, the AR-15512 or a derivative, analogue, racemic mixture, or diastereomer or enantiomer thereof, can be present in an amount of from about 0.0001% to about 5% (w/w), from about 0.0001% to about 0.1% (w/w), from about 0.1% to about 2.5% (w/w), from about 0.1% to about 2% (w/w), from about 0.1% to about 1.5% (w/w), from about 0.10% to about 10% (w/w), from about 0.0010% to about 0.010% (w/w), from about 0.001% to about 0.009% (w/w), from about 0.001% to about 0.008% (w/w), from about 0.001% to about 0.007% (w/w), from about 0.001% to about 0.006% (w/w), from about 0.001% to about 0.005% (w/w), from about 0.001% to about 0.004% (w/w), from about 0.001% to about 0.003% (w/w), from about 0.001% to about 0.002% (w/w), from about 0.0001% to about 0.001% (w/w), from about 0.0001% to about 0.0009% (w/w), from about 0.0001% to about 0.0008% (w/w), from about 0.0001% to about 0.0007% (w/w), from about 0.0001% to about 0.0006% (w/w), from about 0.0001% to about 0.0005% (w/w), from about 0.0001% to about 0.0004% (w/w), from about 0.0001% to about 0.0003% (w/w), from about 0.0001% to about 0.0002% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.10% to about 1.7% (w/w), from about 0.10% to about 1.30% (w/w), from about 0.1% to about 0.5% (w/w), from about 0.1% to about 0.45% (w/w), from about 0.10% to about 0.4% (w/w), from about 0.10% to about 0.35% (w/w), from about 0.1% to about 0.3% (w/w), from about 0.1% to about 0.25% (w/w), from about 0.1% to about 0.2% (w/w), from about 0.15% to about 0.5% (w/w), from about 0.15% to about 0.45% (w/w), from about 0.15% to about 0.4% (w/w), from about 0.15% to about 0.35% (w/w), from about 0.150% to about 0.3% (w/w), from about 0.150% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.30% to about 1.50% (w/w), from about 1.50% to about 2% (w/w), about 0.10% (w/w), about 0.2% (w/w), about 0.30% (w/w), about 0.4% (w/w), about 0.50% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w), about 2.5% (w/w), about 3% (w/w), about 3.5% (w/w), about 4% (w/w), about 4.5% (w/w), about 5% (w/w), about 5.5% (w/w), about 0.001% (w/w), about 0.002% (w/w), about 0.003% (w/w), about 0.004% (w/w), about 0.005% (w/w), about 0.0011% (w/w), about 0.0012% (w/w), about 0.0013% (w/w), about 0.0014% (w/w), about 0.0015% (w/w), about 0.0016% (w/w), about 0.0017% (w/w), about 0.0018% (w/w), about 0.0019% (w/w), about 0.002% (w/w), about 0.0021% (w/w), about 0.0022% (w/w), about 0.0023% (w/w), about 0.0024% (w/w), about 0.0025% (w/w), about 0.0026% (w/w), about 0.0027% (w/w), about 0.0028% (w/w), about 0.0029% (w/w), about 0.0030% (w/w), about 0.0031% (w/w), about 0.0032% (w/w), about 0.0033% (w/w), about 0.0034% (w/w), about 0.0035% (w/w), about 0.0036% (w/w), about 0.0037% (w/w), about 0.0038% (w/w), about 0.0039% (w/w), about 0.0001% (w/w), about 0.0002% (w/w), about 0.0003% (w/w), about 0.0004% (w/w), about 0.0005% (w/w), about 0.0006% (w/w), about 0.0007% (w/w), about 0.0008% (w/w), or about 0.0009% (w/w).

In some instances, provided herein are compositions comprising a therapeutically effective amount of lotilaner, or a derivative, analogue, racemic mixture, or diastereomer or enantiomer thereof as an active pharmaceutical ingredient; a semi-fluorinated alkyl alcohol; and a semi-fluorinated alkane compound. The compositions can, in some instances, be topical ophthalmological compositions.

In some instances, the lotilaner, or a derivative, analogue, racemic mixture, or diastereomer or enantiomer thereof, can be present in an amount of from about 0.0001% to about 5% (w/w), from about 0.1% to about 2.5% (w/w), from about 0.1% to about 2% (w/w), from about 0.10% to about 1.5% (w/w), from about 0.10% to about 10% (w/w), from about 0.001% to about 0.01% (w/w), from about 0.001% to about 0.009% (w/w), from about 0.001% to about 0.008% (w/w), from about 0.001% to about 0.007% (w/w), from about 0.001% to about 0.006% (w/w), from about 0.001% to about 0.005% (w/w), from about 0.001% to about 0.004% (w/w), from about 0.001% to about 0.003% (w/w), from about 0.001% to about 0.002% (w/w), from about 0.0001% to about 0.001% (w/w), from about 0.0001% to about 0.0009% (w/w), from about 0.0001% to about 0.0008% (w/w), from about 0.0001% to about 0.0007% (w/w), from about 0.0001% to about 0.0006% (w/w), from about 0.0001% to about 0.0005% (w/w), from about 0.0001% to about 0.0004% (w/w), from about 0.0001% to about 0.0003% (w/w), from about 0.0001% to about 0.0002% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.10% to about 1.7% (w/w), from about 0.1% to about 1.3% (w/w), from about 0.1% to about 0.5% (w/w), from about 0.1% to about 0.45% (w/w), from about 0.1% to about 0.4% (w/w), from about 0.1% to about 0.35% (w/w), from about 0.10% to about 0.30% (w/w), from about 0.10% to about 0.25% (w/w), from about 0.1% to about 0.2% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.30% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.30% to about 1.30% (w/w), from about 0.30% to about 10% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.10% (w/w), about 0.2% (w/w), about 0.30% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w), about 2.5% (w/w), about 3% (w/w), about 3.5% (w/w), about 4% (w/w), about 4.5% (w/w), about 5% (w/w), about 5.5% (w/w), about 0.001% (w/w), about 0.002% (w/w), about 0.003% (w/w), about 0.004% (w/w), about 0.005% (w/w), about 0.0011% (w/w), about 0.0012% (w/w), about 0.0013% (w/w), about 0.0014% (w/w), about 0.0015% (w/w), about 0.0016% (w/w), about 0.0017% (w/w), about 0.0018% (w/w), about 0.0019% (w/w), about 0.002% (w/w), about 0.0021% (w/w), about 0.0022% (w/w), about 0.0023% (w/w), about 0.0024% (w/w), about 0.0025% (w/w), about 0.0026% (w/w), about 0.0027% (w/w), about 0.0028% (w/w), about 0.0029% (w/w), about 0.0030% (w/w), about 0.0031% (w/w), about 0.0032% (w/w), about 0.0033% (w/w), about 0.0034% (w/w), about 0.0035% (w/w), about 0.0036% (w/w), about 0.0037% (w/w), about 0.0038% (w/w), about 0.0039% (w/w), about 0.0001% (w/w), about 0.0002% (w/w), about 0.0003% (w/w), about 0.0004% (w/w), about 0.0005% (w/w), about 0.0006% (w/w), about 0.0007% (w/w), about 0.0008% (w/w), or about 0.0009% (w/w). In some instances, the lotilaner, or a derivative, analogue, racemic mixture, or diastereomer or enantiomer thereof can be present in the composition in an amount of about 0.25% (w/w).

In some instances, provided herein are compositions comprising a therapeutically effective amount of cyclosporine, or a derivative, analogue, pharmaceutically acceptable salt, free base form, racemic mixture, or diastereomer or enantiomer thereof as an active pharmaceutical ingredient; a semi-fluorinated alkyl alcohol; and a semi-fluorinated alkane compound. The compositions can, in some instances, be topical ophthalmological compositions.

Cyclosporine, or a derivative, analogue, pharmaceutically acceptable salt, free base form, racemic mixture, or diastereomer or enantiomer thereof, can, in some instances, aid in treatment of dry eye syndrome, or keratoconjunctivitis sicca. In some instances, cyclosporine or a derivative, analogue, pharmaceutically acceptable salt, free base form, racemic mixture, or diastereomer or enantiomer thereof, can slow the progression of, treat, or reduce or relieve symptoms of certain ocular diseases or conditions, such as dry eye syndrome or keratoconjunctivitis sicca, dry eye disease, inflammatory dry eye, redness, or meibomian gland disfunction.

In some instances, the cyclosporine or a derivative, analogue, pharmaceutically acceptable salt, free base form, racemic mixture, or diastereomer or enantiomer thereof, can be present in an amount of from about 0.0001% to about 5% (w/w), from about 0.1% to about 2.5% (w/w), from about 0.1% to about 2% (w/w), from about 0.1% to about 1.5% (w/w), from about 0.1% to about 1% (w/w), from about 0.001% to about 0.01% (w/w), from about 0.001% to about 0.009% (w/w), from about 0.001% to about 0.008% (w/w), from about 0.001% to about 0.007% (w/w), from about 0.001% to about 0.006% (w/w), from about 0.001% to about 0.005% (w/w), from about 0.001% to about 0.004% (w/w), from about 0.001% to about 0.003% (w/w), from about 0.001% to about 0.002% (w/w), from about 0.0001% to about 0.001% (w/w), from about 0.0001% to about 0.0009% (w/w), from about 0.0001% to about 0.0008% (w/w), from about 0.0001% to about 0.0007% (w/w), from about 0.0001% to about 0.0006% (w/w), from about 0.0001% to about 0.0005% (w/w), from about 0.0001% to about 0.0004% (w/w), from about 0.0001% to about 0.0003% (w/w), from about 0.0001% to about 0.0002% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.5% (w/w), from about 0.1% to about 0.45% (w/w), from about 0.10% to about 0.4% (w/w), from about 0.10% to about 0.35% (w/w), from about 0.1% to about 0.3% (w/w), from about 0.1% to about 0.25% (w/w), from about 0.1% to about 0.2% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.50% (w/w), from about 0.50% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w), about 2.5% (w/w), about 3% (w/w), about 3.5% (w/w), about 4% (w/w), about 4.5% (w/w), about 5% (w/w), about 5.5% (w/w), about 0.001% (w/w), about 0.002% (w/w), about 0.003% (w/w), about 0.004% (w/w), about 0.005% (w/w), about 0.0011% (w/w), about 0.0012% (w/w), about 0.0013% (w/w), about 0.0014% (w/w), about 0.0015% (w/w), about 0.0016% (w/w), about 0.0017% (w/w), about 0.0018% (w/w), about 0.0019% (w/w), about 0.002% (w/w), about 0.0021% (w/w), about 0.0022% (w/w), about 0.0023% (w/w), about 0.0024% (w/w), about 0.0025% (w/w), about 0.0026% (w/w), about 0.0027% (w/w), about 0.0028% (w/w), about 0.0029% (w/w), about 0.0030% (w/w), about 0.0031% (w/w), about 0.0032% (w/w), about 0.0033% (w/w), about 0.0034% (w/w), about 0.0035% (w/w), about 0.0036% (w/w), about 0.0037% (w/w), about 0.0038% (w/w), about 0.0039% (w/w), about 0.0001% (w/w), about 0.0002% (w/w), about 0.0003% (w/w), about 0.0004% (w/w), about 0.0005% (w/w), about 0.0006% (w/w), about 0.0007% (w/w), about 0.0008% (w/w), or about 0.0009% (w/w). some instances, the cyclosporine, or a derivative, analogue, pharmaceutically acceptable salt, free base form, racemic mixture, or diastereomer or enantiomer thereof can be present in the composition in an amount of about 0.05% (w/w).

In some instances, provided herein are compositions comprising a therapeutically effective amount of a pressure regulator, such as Brimonidine, Apraclonidine Iopidine, Dorzolamide, Brinzolamide, Azopt, Timolol, Timoptic/Istalol/Betimol, Betaxolol, Betoptic, Levobunolol, Betagan, Metipranolol, Optipranolol, Latanoprost, Xalatan Green, Bimatoprost, Lumigan, Tafluprost, Zioptan, Latanoprostene, Vyzulta Turquoise, Pilocarpine, Carbachol, Isopto Carbachol, Netarsudil, and Rhopressa, or a derivative, analogue, pharmaceutically acceptable salt, free base form, racemic mixture, or diastereomer or enantiomer thereof as an active pharmaceutical ingredient; a semi-fluorinated alkyl alcohol; and a semi-fluorinated alkane compound. The compositions can, in some instances, be topical ophthalmological compositions. Pressure regulators, can, in some instances, aid in treatment of glaucoma. In some instances, pressure regulators can slow the progression of, treat, or reduce or relieve symptoms of certain ocular diseases or conditions, such as glaucoma, ocular hypertension, or high fluid pressure in an eye of a subject. In some instances, pressure regulators can lower fluid pressure in an eye of a subject.

In some instances, the pressure regulator, such as Brimonidine or a derivative, analogue, pharmaceutically acceptable salt, free base form, racemic mixture, or diastereomer or enantiomer thereof, can be present in an amount of from about 0.0001% to about 5% (w/w), from about 0.1% to about 2.5% (w/w), from about 0.1% to about 2% (w/w), from about 0.1% to about 1.5% (w/w), from about 0.002% to about 0.3% (w/w), from about 0.0025% to about 0.25% (w/w), from about 0.1% to about 1% (w/w), from about 0.001% to about 0.01% (w/w), from about 0.001% to about 0.009% (w/w), from about 0.001% to about 0.008% (w/w), from about 0.001% to about 0.007% (w/w), from about 0.001% to about 0.006% (w/w), from about 0.001% to about 0.005% (w/w), from about 0.001% to about 0.004% (w/w), from about 0.001% to about 0.003% (w/w), from about 0.001% to about 0.002% (w/w), from about 0.0001% to about 0.001% (w/w), from about 0.0001% to about 0.0009% (w/w), from about 0.0001% to about 0.0008% (w/w), from about 0.0001% to about 0.0007% (w/w), from about 0.0001% to about 0.0006% (w/w), from about 0.0001% to about 0.0005% (w/w), from about 0.0001% to about 0.0004% (w/w), from about 0.0001% to about 0.0003% (w/w), from about 0.0001% to about 0.0002% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.10% to about 1.7% (w/w), from about 0.1% to about 1.3% (w/w), from about 0.1% to about 0.5% (w/w), from about 0.1% to about 0.45% (w/w), from about 0.1% to about 0.4% (w/w), from about 0.1% to about 0.35% (w/w), from about 0.10% to about 0.30% (w/w), from about 0.10% to about 0.25% (w/w), from about 0.1% to about 0.2% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 10% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w), about 2.5% (w/w), about 3% (w/w), about 3.5% (w/w), about 4% (w/w), about 4.5% (w/w), about 5% (w/w), about 5.5% (w/w), about 0.001% (w/w), about 0.002% (w/w), about 0.003% (w/w), about 0.004% (w/w), about 0.005% (w/w), about 0.0011% (w/w), about 0.0012% (w/w), about 0.0013% (w/w), about 0.0014% (w/w), about 0.0015% (w/w), about 0.0016% (w/w), about 0.0017% (w/w), about 0.0018% (w/w), about 0.0019% (w/w), about 0.002% (w/w), about 0.0021% (w/w), about 0.0022% (w/w), about 0.0023% (w/w), about 0.0024% (w/w), about 0.0025% (w/w), about 0.0026% (w/w), about 0.0027% (w/w), about 0.0028% (w/w), about 0.0029% (w/w), about 0.0030% (w/w), about 0.0031% (w/w), about 0.0032% (w/w), about 0.0033% (w/w), about 0.0034% (w/w), about 0.0035% (w/w), about 0.0036% (w/w), about 0.0037% (w/w), about 0.0038% (w/w), about 0.0039% (w/w), about 0.0001% (w/w), about 0.0002% (w/w), about 0.0003% (w/w), about 0.0004% (w/w), about 0.0005% (w/w), about 0.0006% (w/w), about 0.0007% (w/w), about 0.0008% (w/w), or about 0.0009% (w/w).

In some instances, the Brimonidine or a derivative, analogue, pharmaceutically acceptable salt, free base form, racemic mixture, or diastereomer or enantiomer thereof, can be present in an amount of from about 0.0025% to about 0.25% (w/w).

In some instances, provided herein are compositions comprising a therapeutically effective amount of a muscarinic cholinergic receptor agonist as an active pharmaceutical ingredient; a semi-fluorinated alkyl alcohol; and a semi-fluorinated alkane compound. The compositions can, in some instances, be topical ophthalmological compositions.

Muscarinic cholinergic receptor agonists can, in some instances, cause pupil contraction to alter the focusing depth of field of an eye. In some instances, this can temporarily relieve symptoms of certain eye diseases or conditions such as presbyopia. In some instances, through the muscarinic cholinergic receptors, the agonists can lower intraocular pressure (IOP). This mechanism can, in some instances, be used to treat glaucoma. In some instances, the muscarinic cholinergic receptor agonist can be selected from the group consisting of aceclidine, pilocarpine, bethanechol, cevimeline, methacholine, xanomeline, and aprolidine, or combinations thereof. In some instances, the muscarinic cholinergic receptor agonist can be in salt form. In some instances, the muscarinic cholinergic receptor agonist can be in free base form.

In some instances, the muscarinic cholinergic receptor agonist can be present in an amount of from about 0.1% to about 5% (w/w), from about 0.1% to about 2.5% (w/w), from about 0.1% to about 2% (w/w), from about 0.1% to about 1.5% (w/w), from about 0.1% to about 1% (w/w), from about 0.1% to about 0.5% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.50% (w/w), from about 0.50% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), or about 2% (w/w).

In some instances, the muscarinic cholinergic receptor agonist can be aceclidine. In some instances, the aceclidine can be in salt form, such as, for example aceclidine sulfate. In some instances, the aceclidine can be in salt form, such as, for example aceclidine hydrochloride. In some instances, the aceclidine can be in free base form. In some instances, the aceclidine can be present in an amount of from about 0.01% to about 10% (w/w), from about 0.01% to about 9% (w/w), from about 0.01% to about 8% (w/w), from about 0.01% to about 7% (w/w), from about 0.01% to about 6% (w/w), from about 0.01% to about 5% (w/w), from about 0.01% to about 4% (w/w), from about 0.01% to about 3% (w/w), from about 0.01% to about 2% (w/w), from about 0.01% to about 1% (w/w), from about 0.01% to about 0.9% (w/w), from about 0.01% to about 0.8% (w/w), from about 0.01% to about 0.7% (w/w), from about 0.01% to about 0.6% (w/w), from about 0.01% to about 0.5% (w/w), from about 0.01% to about 0.4% (w/w), from about 0.01% to about 0.3% (w/w), from about 0.01% to about 0.2% (w/w), from about 0.01% to about 0.1% (w/w), from about 1% to about 10% (w/w), from about 1% to about 9% (w/w), from about 1% to about 8% (w/w), from about 1% to about 7% (w/w), from about 1% to about 6% (w/w), from about 1% to about 5% (w/w), from about 1% to about 4% (w/w), from about 1% to about 3% (w/w), from about 1% to about 2% (w/w), from about 0.01% to about 1%, 0.01% to about 0.95% (w/w), 0.01% to about 0.9% (w/w), 0.01% to about 0.85% (w/w), 0.01% to about 0.8% (w/w), 0.01% to about 0.75% (w/w), 0.01% to about 0.7% (w/w), 0.010% to about 0.650% (w/w), 0.01% to about 0.6% (w/w), 0.010% to about 0.55% (w/w), 0.01% to about 0.5% (w/w), 0.01% to about 0.45% (w/w), 0.01% to about 0.4% (w/w), 0.01% to about 0.350% (w/w), 0.01% to about 0.30% (w/w), 0.01% to about 0.25% (w/w), 0.010% to about 0.2% (w/w), 0.010% to about 0.150% (w/w), 0.010% to about 0.1% (w/w), 0.01% to about 0.09% (w/w), 0.01% to about 0.08% (w/w), 0.01% to about 0.07% (w/w), 0.01% to about 0.06% (w/w), 0.01% to about 0.05% (w/w), 0.01% to about 0.04% (w/w), 0.01% to about 0.03% (w/w), 0.01% to about 0.02% (w/w), from about 0.1% to about 50% (w/w), from about 0.1% to about 2.5% (w/w), from about 0.1% to about 2% (w/w), from about 0.1% to about 1.5% (w/w), from about 0.1% to about 1% (w/w), from about 0.1% to about 0.5% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), or about 2% (w/w). In some instances, the aceclidine can be present in an amount of from about 0.01% to about 10% (w/w).

In some instances, the muscarinic cholinergic receptor agonist can be pilocarpine. In some instances, the pilocarpine can be in salt form, such as, for example pilocarpine hydrochloride. In some instances, the pilocarpine can be in salt form, such as, for example pilocarpine nitrate. In some instances, the pilocarpine can be in free base form. In some instances, the pilocarpine can be present in an amount of from about 0.1% to about 2% (w/w), from about 0.1% to about 1.5% (w/w), from about 0.1% to about 1% (w/w), from about 0.1% to about 0.5% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.30% to about 1.50% (w/w), from about 1.50% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), or about 2% (w/w).

In some instances, the API can be a tyrosine kinase inhibitor. In some instances, the tyrosine kinase inhibitor can be a multi-kinase inhibitor (MKI) that inhibits one or more selected from the group consisting of Vascular Endothelial Growth Factor Receptors (VEGFR) 1, 2, and 3 and Platelet-derived Growth Factor Receptor (PDGFR), α, β, and Lyn.

In some instances, the API can be a multikinase inhibitor selected from afatinib, amuvatinib, axitinib, cabozantinib, canertinib, cediranib, ceritinib, crenolanib, crizotinib, dabrafenib, dacomitinib, dasatinib, erlotinib, foretinib, gefitinib, golvatinib, ibrutinib, icotinib, idelalisib, imatinib, lapatinib, lenvatinib, neratinib, nilotinib, nintedanib, palbociclib, pazopanib, ponatinib, quizartinib, regorafenib, ruxolitinib, sorafenib, sunitinib, tandutinib, tivantinib, tivozanib, trametinib, vandetanib, vatalanib, vemurafenib, or combinations thereof. In some instances, the multikinase inhibitor can be selected from axitinib, nintedanib, and pazopanib. In some instances, the multikinase inhibitor can be axitinib, and optionally can be present in the compositions described herein in an amount of from about 0.0001% to about 5% (w/w), from about 0.0005% to about 2% (w/w), or from about 0.001% to about 1% (w/w). In some instances, the multikinase inhibitor can be axitinib, and optionally can be present in the compositions described herein in an amount of from about 0.001% to about 1% (w/w). In some instances, the multikinase inhibitor can be nintedanib, and optionally can be present in the compositions described herein in an amount of from about 0.0001% to about 5% (w/w), from about 0.0005% to about 2% (w/w), from about 0.001% to about 1% (w/w), or from about 0.01% to about 1% (w/w). In some instances, the multikinase inhibitor can be nintedanib, and optionally can be present in the compositions described herein in an amount of from about 0.01% to about 1% (w/w). In some instances, the multikinase inhibitor can be pazopanib, and optionally can be present in the compositions described herein in an amount of from about 0.0001% to about 5% (w/w), from about 0.0005% to about 2% (w/w), or from about 0.001% to about 1% (w/w).

In some instances, the API can be present in the compositions described herein in an amount of from about 0.0001% to about 5% (w/w), from about 0.1% to about 2.5% (w/w), from about 0.1% to about 2% (w/w), from about 0.1% to about 1.5% (w/w), from about 0.1% to about 1% (w/w), from about 0.001% to about 0.01% (w/w), from about 0.001% to about 0.009% (w/w), from about 0.001% to about 0.008% (w/w), from about 0.001% to about 0.007% (w/w), from about 0.001% to about 0.006% (w/w), from about 0.001% to about 0.005% (w/w), from about 0.001% to about 0.004% (w/w), from about 0.001% to about 0.003% (w/w), from about 0.001% to about 0.002% (w/w), from about 0.0001% to about 0.001% (w/w), from about 0.0001% to about 0.0009% (w/w), from about 0.0001% to about 0.0008% (w/w), from about 0.0001% to about 0.0007% (w/w), from about 0.0001% to about 0.0006% (w/w), from about 0.0001% to about 0.0005% (w/w), from about 0.0001% to about 0.0004% (w/w), from about 0.0001% to about 0.0003% (w/w), from about 0.0001% to about 0.0002% (w/w), from about 0.15% to about 0.5% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.5% (w/w), from about 0.1% to about 0.45% (w/w), from about 0.1% to about 0.4% (w/w), from about 0.10% to about 0.35% (w/w), from about 0.10% to about 0.3% (w/w), from about 0.1% to about 0.25% (w/w), from about 0.1% to about 0.2% (w/w), from about 0.15% to about 0.50% (w/w), from about 0.15% to about 0.45% (w/w), from about 0.15% to about 0.4% (w/w), from about 0.15% to about 0.35% (w/w), from about 0.15% to about 0.3% (w/w), from about 0.15% to about 0.25% (w/w), from about 0.15% to about 0.2% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w), about 2.5% (w/w), about 3% (w/w), about 3.5% (w/w), about 4% (w/w), about 4.5% (w/w), about 5% (w/w), about 5.5% (w/w), about 0.001% (w/w), about 0.002% (w/w), about 0.003% (w/w), about 0.004% (w/w), about 0.005% (w/w), about 0.0011% (w/w), about 0.0012% (w/w), about 0.0013% (w/w), about 0.0014% (w/w), about 0.0015% (w/w), about 0.0016% (w/w), about 0.0017% (w/w), about 0.0018% (w/w), about 0.0019% (w/w), about 0.002% (w/w), about 0.0021% (w/w), about 0.0022% (w/w), about 0.0023% (w/w), about 0.0024% (w/w), about 0.0025% (w/w), about 0.0026% (w/w), about 0.0027% (w/w), about 0.0028% (w/w), about 0.0029% (w/w), about 0.0030% (w/w), about 0.0031% (w/w), about 0.0032% (w/w), about 0.0033% (w/w), about 0.0034% (w/w), about 0.0035% (w/w), about 0.0036% (w/w), about 0.0037% (w/w), about 0.0038% (w/w), about 0.0039% (w/w), about 0.0001% (w/w), about 0.0002% (w/w), about 0.0003% (w/w), about 0.0004% (w/w), about 0.0005% (w/w), about 0.0006% (w/w), about 0.0007% (w/w), about 0.0008% (w/w), or about 0.0009% (w/w).

The compositions described herein can include a semifluorinated alkane (SFA). A semifluorinated alkane is an amphiphilic liquid with two mutually immiscible moieties (hydrocarbon segment and perfluorinated segment) bound covalently. In some instances, the semifluorinated alkane can be a partially fluorinated alkane compound having a formula CF₃(CF₂)m(CH₂)ₙCH₃. In some instances, m is an integer from 1 to 10, and n is an integer from 1 to 10. For example, m is can be 1, 2, 3, 4, 5, or 6. In another example, n can be 1, 2, 3, 4, 5, or 6. Suitable examples of semifluorinated alkane compounds include perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10). In some instances, the SFA is perfluorohexyloctane (F6H8).

The structure of F6H8 is shown below:

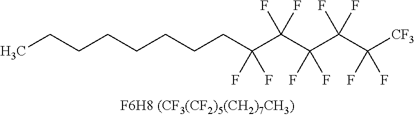 [SMILES: CCCCCCCCC(F)(F)C(F)(F)C(F)(F)C(F)(F)C(F)(F)C(F)(F)F]

In some instances, the SFA can be present in the compositions in an amount of from about 50% to about 99% (w/w), from about 60% to about 99% (w/w), from about 65% to about 99% (w/w), from about 70% to about 99% (w/w), from about 75% to about 99% (w/w), from about 80% to about 99% (w/w), from about 85% to about 99% (w/w), from about 90% to about 99% (w/w), from about 95% to about 99% (w/w), from about 50% to about 90% (w/w), from about 60% to about 90% (w/w), from about 70% to about 90% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 89% (w/w), about 88% (w/w), about 87% (w/w), about 86% (w/w), about 85% (w/w), about 80% (w/w), about 75% (w/w), about 70% (w/w), about 65% (w/w), about 60% (w/w), about 55% (w/w), or about 50% (w/w).

The compositions described herein can include medium chain triglycerides (MCT). Medium-chain triglycerides (MCTs) are triglycerides of fatty acids. The fatty acids have an aliphatic chain of 6-12 carbon atoms, and can be, for example, hexanoic acid, octanoic acid, decanoic acid, and dodecanoic acid. The MCTs can be a single triglyceride or a mixture of triglycerides. Representative chemical structures of the MCTs are shown below.

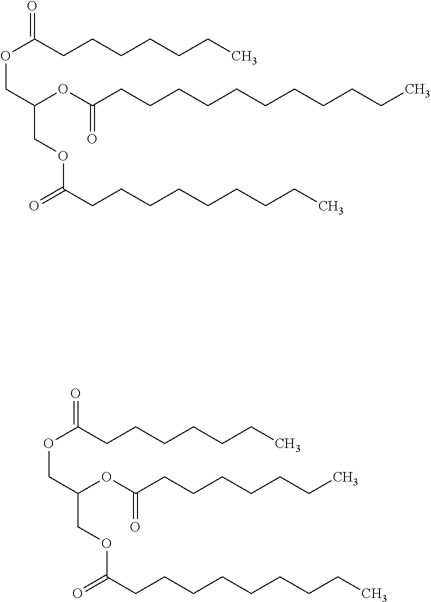 [SMILES: CCCCCCCCCCCC(=O)OC(COC(=O)CCCCCCC)COC(=O)CCCCCCCCC ; CCCCCCCCCC(=O)OCC(COC(=O)CCCCCCC)OC(=O)CCCCCCC]

In some instances, the fatty acids in the MCT can comprise hexanoic acid, octanoic acid, decanoic acid, dodecanoic acid or combinations of two or more thereof. In some instances, the fatty acids in the MCT consist essentially of hexanoic acid, octanoic acid, decanoic acid, dodecanoic acid or combinations thereof.

In some embodiments, the MCT can be present in the compositions in an amount of from about 0.1% to about 50% (w/w), from about 0.1% to about 10% (w/w), from about 1% to about 30% (w/w), from about 1% to about 25% (w/w), from about 1% to about 20% (w/w), from about 1% to about 15% (w/w), from about 1% to about 10% (w/w), from about 5% to about 20% (w/w), from about 5% to about 15% (w/w), from about 10% to about 15% (w/w), from about 10% to about 20% (w/w), from about 10% to about 30% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), or about 1% (w/w). In some embodiments, the MCT can be present in the compositions in an amount of from about 5% to about 20% (w/w) MCT. In some embodiments, the MCT can be present in the compositions in an amount of from about 0.1% to about 10% (w/w) MCT. Applicant has discovered that MCT in these amounts offers a balance of ocular tolerability, such as non-irritation, along with, in some instances, desired solubility of the API. In some instances of the nano-emulsion compositions provided herein, Applicant has discovered that these amounts of MCT also balance the desired droplet size with the ocular tolerability of the compositions.

The compositions disclosed herein may, in some instances, also comprise one or more additional therapeutic agents, excipients, or diluents including, but not limited to, absorbents, anti-irritants, preservatives, antioxidants, coloring agents/pigments, emollients (moisturizers), emulsifiers, film-forming/holding agents, prescription drugs, surfactants/detergent cleansing agents, penetration enhancers, viscosity enhancers, and thickeners.

The compositions of the present application can include those suitable for any acceptable route of administration. Suitable examples of acceptable routes of administration include ocular, topical ocular, ocular injection, and the like. In some embodiments, the topical composition is an ophthalmic composition, e.g., for intraocular administration. Suitable examples of ophthalmic compositions include eye drops, emulsions, nanoemulsions, and oils. Any one of these ophthalmic compositions can be administered directly to the cornea or surface of the eye, using, for example, a plastic or latex applicator (e.g., a single-use applicator), an eye dropper, a glass pipette, or a rubber bulb.

In some instances, the composition has a pH of about 6 to about 8.

In some instances, the compositions (e.g., topical ophthalmic compositions or topical ophthalmic nano-emulsions) of the present application are absorbed transcorneally or are selectively absorbed transcorneally. The phrases “absorbed via the transcorneal route,” “absorbed via transcorneal pathway,” and “absorbed transcorneally” are used interchangeably and relate to transcorneal absorption. In transcorneal absorption, a drug typically penetrates the eye through cornea to the aqueous humor to the iris. In the periorbital absorption, drug typically penetrates the eye through the conjunctival to the scleral to the ciliary body. In some instances, a composition described herein is absorbed transcorneally (e.g., at least 5%, at least 10%, at least 20%, at least 30%, at least 40%, at least 50%, at least 60%, or at least 70% of the composition is absorbed transcorneally, e.g., as determined by an assay known in the art or described in the working examples herein). In some instances, a composition described herein is selectively absorbed transcorneally (e.g., at least 5%, at least 10%, at least 20%, at least 30%, at least 40%, at least 50%, at least 60%, or at least 70% more of the composition is absorbed transcorneally as compared to the amount absorbed periorbitally, e.g., as determined by an assay known in the art or described in the working examples herein, or at least 1.5-fold, at least 2-fold, at least 3-fold, at least 4-fold, at least 5-fold, at least 10-fold, or at least 20-fold more of the composition is absorbed transcorneally as compared to the amount absorbed periorbitally, e.g., as determined by an assay known in the art or described in the working examples herein). In some instances, the amount of the composition that is absorbed transcorneally is at least 5%, at least 10%, at least 20%, at least 30%, at least 40%, at least 50%, at least 60%, or at least 70% greater than the amount that is absorbed periorbitally.

In some instances, the API of a composition of the present application is absorbed transcorneally or is selectively absorbed transcorneally. In some instances, an API described herein is absorbed transcorneally (e.g., at least 5%, at least 10%, at least 20%, at least 30%, at least 40%, at least 50%, at least 60%, or at least 70% of the API is absorbed transcorneally, e.g., as determined by an assay known in the art or described in the working examples herein). In some instances, an API described herein is selectively absorbed transcorneally (e.g., at least 5%, at least 10%, at least 20%, at least 30%, at least 40%, at least 50%, at least 60%, or at least 70% more of the API is absorbed transcorneally as compared to the amount absorbed periorbitally, e.g., as determined by an assay known in the art or described in the working examples herein, or at least 1.5-fold, at least 2-fold, at least 3-fold, at least 4-fold, at least 5-fold, at least 10-fold, or at least 20-fold more of the API is absorbed transcorneally as compared to the amount absorbed periorbitally, e.g., as determined by an assay known in the art or described in the working examples herein). In some instances, the amount of the API that is absorbed transcorneally is at least 5%, at least 10%, at least 20%, at least 30%, at least 40%, at least 50%, at least 60%, or at least 70% greater than the amount that is absorbed periorbitally.

In some instances, the compositions are prepared by uniformly and intimately bringing into association the API as disclosed herein with the MCT and SFA as disclosed herein.

In some instances, the compositions can be in the form of an organic composition without aqueous components.

This disclosure also provides nano-emulsions as described herein and methods for making the nano-emulsions. Applicant has surprisingly discovered that it is possible to form nano-emulsions of a composition described herein, such as compositions comprising an API, MCT, and SFA. In some instances, the nano-emulsions can be oil-in-oil emulsions. In some instances, the nano-emulsions can have a droplet size D90 of less than 100 nm.

“Droplet size” of an emulsion refers to the peak droplet size or the mean droplet size, of the dispersed phase. “Droplet size D90” refers to the case that 90% of the particles have a size smaller than the indicated size. Droplet size can be measured by methods known in the art, e.g., by light scattering after dilution in water using a High-Performance Particle Sizer.

In some instances, the nano-emulsions can have a droplet size D90 of less than about 50 nm, less than about 50 nm, less than about 50 nm, less than about 50 nm, less than about 50 nm, less than about 50 nm, less than about 50 nm, or less than about 50 nm, or from about 1 to 20 nm, from about 1 to 10 nm, from about 1 to 5 nm, or from about 2 to 3 nm. Without being bound by theory, it is believed that the smaller oil droplet particle sizes can increase surface area and residential time of the API in ocular tissues, thus increases API concentrations in ocular tissues and increasing efficacy at the target tissue and decreasing systemic exposure to the API.

In some instances, the methods for making the nano emulsions can comprise combining an ocular API with MCT to form an MCT solution; combining said MCT solution with a semi-fluorinated alkane compound to form a mixture, wherein said mixture comprises from about 1% to about 50% (w/w) said MCT solution and from about 50% to about 99% (w/w) of said semi-fluorinated alkane compound; and emulsifying said mixture and reducing droplet particle size D90 within said mixture to less than 100 nm by physical agitation selected from high speed stirring, vortexing, sonicating, heating and stirring, or homogenizing the mixture until the droplet size reaches droplet particle size D90 of less than 100 nm, less than about 50 nm, less than about 50 nm, less than about 50 nm, less than about 50 nm, less than about 50 nm, less than about 50 nm, less than about 50 nm, or less than about 50 nm, or from about 1 to 20 nm, from about 1 to 10 nm, from about 1 to 5 nm, or from about 2 to 3 nm. In some instances, the methods for making the nano emulsions can comprise uniformly and intimately bringing into association the API as disclosed herein with the MCT and SFA as disclosed herein, and emulsifying said mixture and reducing droplet particle size D90 within said mixture to less than 100 nm by physical agitation as described herein.

In some instances, the methods can further comprise measuring the droplet particle size of the mixture during or following said physical agitation.

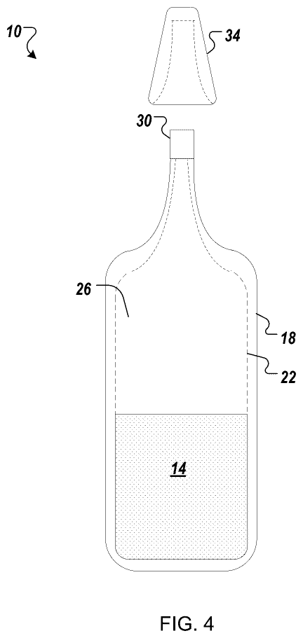

Also provided herein are dispensers comprising the compositions or nano-emulsions described herein. In one example of FIG. 4, an example dispenser 10 contains a liquid 14 of the compositions or nano-emulsions. Applicant surprisingly discovered that the compositions and nanoemulsions described herein are less stable in low density polyethylene (LDPE) and in polypropylene (PP) than in other materials such as glass or polyethylene terephthalate (PET). Without being bound by theory it is believed that LDPE and PET material absorb one or more components of the compositions or nano-emulsions described herein, thereby reducing the content of the active pharmaceutical ingredient (API) over time.

The dispenser 10 includes a container portion 18 having an interior wall 22 defining an interior volume 26 and adapted to contact and store a liquid 14 within at least a portion of the interior volume 26; a dispensing portion 30 adapted to dispense one or more drops of a liquid 14 out of the container portion 18; and, contained within the interior volume 26, a liquid 14 comprising compositions or nano-emulsion as described herein, or a composition comprising about 0.001% to about 5% (w/w) API, wherein the API has a solubility in MCT of at least from about 0.01% to about 10% (w/w); from about 1% to about 50% (w/w) of a medium chain triglyceride (MCT); and from about 50% to about 99% (w/w) of a semi-fluorinated alkane compound.

In some instances, the interior wall 22 of the dispenser 10 comprises less than about 10%, less than about 5%, or less than about 1% of one or more of low density polyethylene (LDPE) or polypropylene (PP). In some instances, the interior wall 22 does not comprise low density polyethylene (LDPE) or polypropylene (PP). In some instances, the interior wall 22 comprises glass or polyethylene terephthalate. In some instances, the interior wall 22 consists essentially of glass or polyethylene terephthalate (PET).

In some instances, the concentration of the API in the compositions or nano-emulsions of the liquid 14 remains more than 90% of the initial API concentration after storage in the dispenser 10 for a period of 6 months. In some instances, the concentration of the API in the compositions or nano-emulsions of the liquid 14 remains more than 90% of the initial API concentration after storage in the dispenser 10 for a period of 12 months.

In some instances, the container portion 18 and the dispensing portion 30 can each, independently, comprise or consist of the same material or different materials. In some instances, the container portion 18 and the dispensing portion 30 can each, independently, consist essentially of glass or polyethylene terephthalate (PET). In some instances, the dispensing portion 30 can comprise low density polyethylene (LDPE) or polypropylene (PP), particularly where the compositions or nano-emulsions of the liquid 14 do not typically come into contact with the dispensing portion 30 during normal storage of the compositions or nano-emulsions of the liquid 14, but come into contact with the dispensing portion 30 briefly during dispensation, such as when a drop is passing through the dispensing portion 30 from the container portion 18 during administration to an eye of a subject. Without being bound by theory, it is believed such limited contact of the compositions or nano-emulsions of the liquid 14 with LDPE or PET will have little or no effect on the compositions or nano-emulsions of the liquid 14.

In some instances, the dispenser 10 is adapted to dispense drops each independently having a volume of from about 5 μl to about 40 μl.

The compositions and nano-emulsions described herein can be conveniently presented in a unit dosage form or a multi-dosage form (e.g., the dispenser 10). In some embodiments, compositions and formulations described herein can be conveniently presented in a dosage form (unit or multiple) that can be used with or in an automated dosing apparatus or regimen.

In some instances, the dispenser 10 may optionally have a cap 34 that is removable from the container portion 18. For example, the cap 34 can cover the dispensing portion 30 during storage.

This disclosure also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

In some embodiments, a kit can comprise a dispensing device preloaded with a composition as described herein.

Also provided herein are methods for treating ocular diseases or conditions using the compositions, dispensers, and methods described herein. In some instances, the ocular diseases or conditions can be related to the cornea or corneal tissues. In some instances, the ocular disease or condition is preferably treated with a composition that is absorbed (e.g., selectively absorbed) transcorneally.

Exemplary, nonlimiting ocular diseases that can be treated with the compositions, dispensers, and methods described herein include chemical burns or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatricizing disease such as Ocular Cicatricial Pemphigoid, Stevens Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD).

In some instances, the disease or condition can be a front of eye disease or condition. Exemplary, nonlimiting front of eye diseases or conditions that can be treated with the compositions, dispensers, and methods described herein include neurotrophic keratitis, glaucoma, elevated intraocular pressure, ocular hypertension, presbyopia, myopia, ocular rosacea, dry eye disease, meibomian gland dysfunction, blepharitis, allergic conjunctivitis, atopic keratoconjunctivitis, vernal keratoconjunctivitis, pterygium, pinguecula, corneal transplant rejection, graft versus host disease, ocular allergy, uveitis, anterior uveitis, Behcet's disease, Sjogren's syndrome, Stevens-Johnson syndrome, ocular cicatricial pemphigoid, chronic ocular surface inflammation caused by viral infection, herpes simplex keratitis, atopic conjunctivitis, Lyell's syndrome, neovascularization induced by viral, bacterial, fungal, or parasitic infection, contact lens induced neovascularization, ulceration, alkali burns, stem cell deficiency, neovascular glaucoma, Steven Johnson syndrome, tumor in the eye, aphakia, pseudophakia, astigmatism, blepharospasm, cataract, conjunctival diseases, corneal diseases, corneal ulcer, eyelid diseases, lacrimal apparatus diseases, pupil disorders, refractive disorders, strabismus.

In some instances, the disease or condition can be a back of eye disease or condition.

In some instances the methods can include a method for treating, slowing the progression of, or reducing one or more symptoms of an ocular condition in a subject comprising administering the topical ophthalmological compositions or the nano-emulsion compositions described herein to the eye of a subject. In some instances the methods can include a method of delivering an API to an eye of a subject comprising administering the topical ophthalmological compositions or the nano-emulsion compositions described herein. Administering can in some instances include contacting the eye with the composition.

In some instances the methods can include a method for slowing myopia progression in a subject or for relieving vitreous floater symptoms in a subject, comprising administering the topical ophthalmological compositions or the nano-emulsion compositions described herein to the subject, wherein the API is a muscarinic receptor antagonist, such as, for example, atropine, an atropine mimetic or analog, or a free base or salt form thereof.

In some instances, the methods further comprise (a) (i) selecting a subject in need of transcorneal absorption of an API; or (ii) selecting a subject having an ocular condition, wherein the ocular condition is in need of transcorneal absorption of an API.

In some instances of the methods, the API is selectively delivered to the cornea of the subject.

In some instances of the methods, the subject has failed to respond to treatment with an aqueous composition comprising the API of the composition or a salt thereof. Failure to respond to treatment includes disease progression and failure to achieve clinically significant symptom reduction(s) after treatment.

In some instances of the methods, the subject has had an adverse side effect from the API after receiving an aqueous composition comprising the API. In some instances of the methods, the subject has had an adverse side effect from the API after receiving an aqueous composition comprising a salt of the API. An adverse side effect from the API includes adverse side effects attributed to the API and not to other components or features of a composition comprising the API (e.g., a preservative or pH). Adverse side effects include headache and ocular irritation.

In some instances of the methods, the subject is highly sensitive to an aqueous composition comprising the API or a salt thereof.

In some instances, the API can be present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician. For example, in some embodiments, where the compositions described herein may be used to treat a disease in an eye of the patient, such as myopia, the treating physician may adjust the dosage and dosage regimen based on the current progression of the disease in the patient, the patient's age, or other related factors.

In some instances, the API is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of the API when administered as an aqueous topical ophthalmological composition for the same method of use. The decrease in dose is not due to differences in weight between free base form and acid form of an API. For example, in some instances of a method of treating an ocular condition in a subject, the API is administered to the subject at a dose at least 30% less than a dose of the API in an aqueous topical ophthalmological composition for treating the ocular condition.

In some instances, the API dosages can be administered on a daily basis (e.g., as a single dose or as two or more divided doses, e.g., once daily, two times daily, three times daily, or four times daily) or non-daily basis (e.g., every other day, every two days, every three days, once weekly, twice weekly, once every two weeks, once a month). In some instances, the API dosages can be administered once every about 6 to about 8 hours. In some instances, the API dosages can be administered three times a day. In some instances, the API dosages can be administered once a day.

Also provided herein are compositions comprising: a therapeutically effective amount of a muscarinic cholinergic receptor agonist as an active pharmaceutical ingredient; and a semifluorinated alkane compound. The semifluorinated alkane compound can, in some embodiments be the liquid vehicle of the composition. In some embodiments, the composition can further comprise an organic cosolvent. The compositions can provide stable formulations for muscarinic cholinergic receptor agonists.

In some instances, provided herein are compositions comprising a therapeutically effective amount of a muscarinic cholinergic receptor agonist as an active pharmaceutical ingredient; and a semifluorinated alkane compound. The compositions can, in some instances, be topical ophthalmological compositions.

Muscarinic cholinergic receptor agonists can, in some instances, cause pupil contraction to alter the focusing depth of field of an eye. In some instances, this can temporarily relieve symptoms of certain eye diseases or conditions such as presbyopia. In some instances, through the muscarinic cholinergic receptors, the agonists can lower intraocular pressure (IOP). This mechanism can, in some instances, be used to treat glaucoma. In some instances, the muscarinic cholinergic receptor agonist can be selected from the group consisting of aceclidine, pilocarpine, bethanechol, cevimeline, methacholine, xanomeline, and aprolidine, or combinations thereof. In some instances, the muscarinic cholinergic receptor agonist can be in salt form. In some instances, the muscarinic cholinergic receptor agonist can be in free base form.

In some instances, the muscarinic cholinergic receptor agonist can be present in an amount of from 0.1% to about 5% (w/w), from about 0.1% to about 2.5% (w/w), from about 0.1% to about 2% (w/w), from about 0.1% to about 1.5% (w/w), from about 0.1% to about 1% (w/w), from about 0.1% to about 0.5% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w).

In some instances, the muscarinic cholinergic receptor agonist can be aceclidine. In some instances, the aceclidine can be in salt form, such as, for example aceclidine sulfate. In some instances, the aceclidine can be in salt form, such as, for example aceclidine hydrochloride. In some instances, the aceclidine can be in free base form. In some instances, the aceclidine can be present in an amount of from 0.1% to about 5% (w/w), from about 0.10% to about 2.5% (w/w), from about 0.10% to about 2% (w/w), from about 0.1% to about 1.5% (w/w), from about 0.1% to about 1% (w/w), from about 0.1% to about 0.5% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.5% (w/w), from about 0.5% to about 2% (w/w), from about 0.5% to about 0.7% (w/w), from about 0.7% to about 1% (w/w), from about 1% to about 1.3% (w/w), from about 1.3% to about 1.5% (w/w), from about 1.5% to about 2% (w/w), about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w).

In some instances, the muscarinic cholinergic receptor agonist can be pilocarpine. In some instances, the pilocarpine can be in salt form, such as, for example pilocarpine hydrochloride. In some instances, the pilocarpine can be in salt form, such as, for example pilocarpine nitrate. In some instances, the pilocarpine can be in free base form. In some instances, the pilocarpine can be present in an amount of from about 0.1% to about 2% (w/w), from about 0.1% to about 1.5% (w/w), from about 0.1% to about 1% (w/w), from about 0.1% to about 0.5% (w/w), from about 0.1% to about 1.7% (w/w), from about 0.1% to about 1.3% (w/w), from about 0.1% to about 0.7% (w/w), from about 0.1% to about 2% (w/w), from about 0.3% to about 2% (w/w), from about 0.5% to about 2% (w/w), from about 0.7% to about 2% (w/w), from about 1% to about 2% (w/w), from about 1.3% to about 2% (w/w), from about 1.5% to about 2% (w/w), from about 1.7% to about 2% (w/w), from about 0.3% to about 1% (w/w), from about 0.3% to about 1.3% (w/w), from about 0.3% to about 1% (w/w), from about 0.5% to about 1% (w/w), from about 0.5% to about 0.7% (w/w), from about 0.5% to about 1% (w/w), from about 0.5 to about 1.50% (w/w), from about 0.50% to about 2% (w/w), from about 0.5% to about 0.7% (w/w), from about 0.7% to about 1% (w/w), from about 1% to about 1.3% (w/w), from about 1.3% to about 1.50% (w/w), from about 1.5% to about 2% (w/w), about 0.10% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.3% (w/w), about 1.5% (w/w), about 1.7% (w/w), about 2% (w/w) In some instances, the compositions comprise a semifluorinated alkane compound. The semifluorinated alkane compound can act, in some instances, as a liquid vehicle. A semifluorinated alkane compound is an amphiphilic liquid with two mutually immiscible moieties (hydrocarbon segment and perfluorinated segment) bound covalently. The semi-fluorinated alkane compound can, in some instances, have a formula of RFRH or a formula of RFRHRF; RF is a perfluorinated hydrocarbon with 1 to 15 carbon atoms, and wherein RH is a non-fluorinated hydrocarbon with 1 to 15 carbon atoms.

Examples of semifluorinated alkanes that can be useful in the compositions described herein include perfluorobutylpentane (F4H5), perfluorobutylhexane (F4H6), perfluorohexylbutane (F6H4), perfluorohexylhexane (F6H6), perfluorohexyloctane (F6H8), and perfluorohexyldecane (F6H10); preferably, perfluorobutylpentane (F4H5), perfluorohexylhexane (F6H6), and perfluorohexyloctane (F6H8).

The structure of F6H8 is shown below.

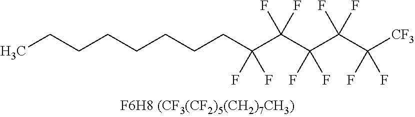 [SMILES: CCCCCCCCC(F)(F)C(F)(F)C(F)(F)C(F)(F)C(F)(F)C(F)(F)F]

In some instances, the semifluorinated alkane can be present in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

In some instances, the semifluorinated alkane is F6H8 (perfluorohexyloctane). In some instances, the semifluorinated alkane is perfluorobutylheptane (F4H5).

In some instances, the compositions can further comprise one or more organic cosolvents. The cosolvent can, in some instances, work to further improve solubility of the muscarinic cholinergic receptor agonist. The organic cosolvent can, in some instances, be selected from the group consisting of medium-chain triglycerides (MCT), liquid paraffin, vitamin E acetate, D-a-tocopherol, oleic acid, ethyl oleate and combinations thereof.

In some instances, the organic co-solvent can be present in an amount of from about 1% to about 50% (w/w), from about 1% to about 50% (w/w), from about 5% to about 50% (w/w), from about 10% to about 50% (w/w), from about 15% to about 50% (w/w), from about 20% to about 50% (w/w), from about 25% to about 50% (w/w), from about 30% to about 50% (w/w), from about 30% to about 50% (w/w), from about 35% to about 50% (w/w), from about 40% to about 50% (w/w), from about 45% to about 50% (w/w), from about 1% to about 50% (w/w), from about 1% to about 45% (w/w), from about 1% to about 40% (w/w), from about 1% to about 35% (w/w), from about 1% to about 30% (w/w), from about 1% to about 25% (w/w), from about 1% to about 20% (w/w), from about 1% to about 15% (w/w), from about 1% to about 10% (w/w), from about 1% to about 5% (w/w), from about 3% to about 5% (w/w), from about 5% to about 7% (w/w), from about 7% to about 9% (w/w), from about 9% to about 11% (w/w), from about 11% to about 13% (w/w), from about 13% to about 15% (w/w), from about 1% to about 3% (w/w), from about 15% to about 20% (w/w), from about 20% to about 25% (w/w), from about 25% to about 30% (w/w), about 15% (w/w), about 14% (w/w), about 13% (w/w), about 12% (w/w), about 11% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w).

In some instances, the compositions can comprise a combination of one or more organic cosolvents as described herein and one or more SFAs as described herein. In some instances, the cosolvent can be present in an amount of about 1% (w/w) and the SFA can be present in an amount of about 99% (w/w); the cosolvent can be present in an amount of about 2% (w/w) and the SFA can be present in an amount of about 98% (w/w); the cosolvent can be present in an amount of about 5% (w/w) and the SFA can be present in an amount of about 95% (w/w); the cosolvent can be present in an amount of about 7% (w/w) and the SFA can be present in an amount of about 93% (w/w); the cosolvent can be present in an amount of about 10% (w/w) and the SFA can be present in an amount of about 90% (w/w); the cosolvent can be present in an amount of about 13% (w/w) and the SFA can be present in an amount of about 87% (w/w); the cosolvent can be present in an amount of about 15% (w/w) and the SFA can be present in an amount of about 85% (w/w); the cosolvent can be present in an amount of about 17% (w/w) and the SFA can be present in an amount of about 83% (w/w); the cosolvent can be present in an amount of about 20% (w/w) and the SFA can be present in an amount of about 80% (w/w); the cosolvent can be present in an amount of about 23% (w/w) and the SFA can be present in an amount of about 77% (w/w); the cosolvent can be present in an amount of about 25% (w/w) and the SFA can be present in an amount of about 75% (w/w); the cosolvent can be present in an amount of about 27% (w/w) and the SFA can be present in an amount of about 73% (w/w); the cosolvent can be present in an amount of about 30% (w/w) and the SFA can be present in an amount of about 70% (w/w); the cosolvent can be present in an amount of about 33% (w/w) and the SFA can be present in an amount of about 67% (w/w); the cosolvent can be present in an amount of about 35% (w/w) and the SFA can be present in an amount of about 65% (w/w); the cosolvent can be present in an amount of about 37% (w/w) and the SFA can be present in an amount of about 63% (w/w); the cosolvent can be present in an amount of about 40% (w/w) and the SFA can be present in an amount of about 60% (w/w); the cosolvent can be present in an amount of about 43% (w/w) and the SFA can be present in an amount of about 57% (w/w); the cosolvent can be present in an amount of about 45% (w/w) and the SFA can be present in an amount of about 55% (w/w); the cosolvent can be present in an amount of about 47% (w/w) and the SFA can be present in an amount of about 53% (w/w); the cosolvent can be present in an amount of about 50% (w/w) and the SFA can be present in an amount of about 50% (w/w); SFA can be the only vehicle and the SFA can be present in an amount of about 98% (w/w); SFA can be the only vehicle and the SFA can be present in an amount of about 98.5% (w/w); SFA can be the only vehicle and the SFA can be present in an amount of about 98.6% (w/w); SFA can be the only vehicle and the SFA can be present in an amount of about 98.7% (w/w); SFA can be the only vehicle and the SFA can be present in an amount of about 98.8% (w/w); or SFA can be the only vehicle and the SFA can be present in an amount of about 98.9% (w/w).

In some instances, the concentration of the organic cosolvent in the composition is less than or equal to about 50% (w/w). In some instances, the concentration of organic cosolvent in the composition is from about 1% to 15% (w/w).

In some instances, the organic cosolvent can be medium-chain triglycerides (MCT). In some instances, the medium-chain triglycerides (MCT) can have two or three fatty acids each independently having an aliphatic tail of 6-12 carbon atoms.

In some instances, the MCT can be present in an amount of from about 1% to about 50% (w/w), from about 1% to about 50% (w/w), from about 5% to about 50% (w/w), from about 10% to about 50% (w/w), from about 15% to about 50% (w/w), from about 20% to about 50% (w/w), from about 25% to about 50% (w/w), from about 30% to about 50% (w/w), from about 30% to about 50% (w/w), from about 35% to about 50% (w/w), from about 40% to about 50% (w/w), from about 45% to about 50% (w/w), from about 1% to about 50% (w/w), from about 1% to about 45% (w/w), from about 1% to about 40% (w/w), from about 1% to about 35% (w/w), from about 1% to about 30% (w/w), from about 1% to about 25% (w/w), from about 1% to about 20% (w/w), from about 1% to about 15% (w/w), from about 1% to about 10% (w/w), from about 1% to about 50% (w/w), from about 30% to about 50% (w/w), from about 50% to about 7% (w/w), from about 7% to about 9% (w/w), from about 9% to about 11% (w/w), from about 11% to about 13% (w/w), from about 13% to about 15% (w/w), from about 1% to about 3% (w/w), from about 15% to about 20% (w/w), from about 20% to about 25% (w/w), from about 25% to about 30% (w/w), about 15% (w/w), about 14% (w/w), about 13% (w/w), about 12% (w/w), about 110% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w)).

In some instances, the concentration of the MCT in the composition is less than or equal to about 50% (w/w). In some instances, the concentration of the MCT in the composition is from about 1% to 15% (w/w).

In some instances, the compositions can comprise a combination of one or more MCTs as described herein and one or more SFAs as described herein. In some instances, the MCT can be present in an amount of about 1% (w/w) and the SFA can be present in an amount of about 99% (w/w); the MCT can be present in an amount of about 2% (w/w) and the SFA can be present in an amount of about 98% (w/w); the MCT can be present in an amount of about 5% (w/w) and the SFA can be present in an amount of about 95% (w/w); the MCT can be present in an amount of about 7% (w/w) and the SFA can be present in an amount of about 93% (w/w); the MCT can be present in an amount of about 10% (w/w) and the SFA can be present in an amount of about 90% (w/w); the MCT can be present in an amount of about 13% (w/w) and the SFA can be present in an amount of about 87% (w/w); the MCT can be present in an amount of about 15% (w/w) and the SFA can be present in an amount of about 85% (w/w); the MCT can be present in an amount of about 17% (w/w) and the SFA can be present in an amount of about 83% (w/w); the MCT can be present in an amount of about 20% (w/w) and the SFA can be present in an amount of about 80% (w/w); the MCT can be present in an amount of about 23% (w/w) and the SFA can be present in an amount of about 77% (w/w); the MCT can be present in an amount of about 25% (w/w) and the SFA can be present in an amount of about 75% (w/w); the MCT can be present in an amount of about 27% (w/w) and the SFA can be present in an amount of about 73% (w/w); the MCT can be present in an amount of about 30% (w/w) and the SFA can be present in an amount of about 70% (w/w); the MCT can be present in an amount of about 33% (w/w) and the SFA can be present in an amount of about 67% (w/w); the MCT can be present in an amount of about 35% (w/w) and the SFA can be present in an amount of about 65% (w/w); the MCT can be present in an amount of about 37% (w/w) and the SFA can be present in an amount of about 63% (w/w); the MCT can be present in an amount of about 40% (w/w) and the SFA can be present in an amount of about 60% (w/w); the MCT can be present in an amount of about 43% (w/w) and the SFA can be present in an amount of about 57% (w/w); the MCT can be present in an amount of about 45% (w/w) and the SFA can be present in an amount of about 55% (w/w); the MCT can be present in an amount of about 47% (w/w) and the SFA can be present in an amount of about 53% (w/w); the MCT can be present in an amount of about 50% (w/w) and the SFA can be present in an amount of about 50% (w/w); SFA can be the only vehicle and the SFA can be present in an amount of about 98% (w/w); SFA can be the only vehicle and the SFA can be present in an amount of about 98.5% (w/w); SFA can be the only vehicle and the SFA can be present in an amount of about 98.6% (w/w); SFA can be the only vehicle and the SFA can be present in an amount of about 98.7% (w/w); SFA can be the only vehicle and the SFA can be present in an amount of about 98.8% (w/w); or SFA can be the only vehicle and the SFA can be present in an amount of about 98.9% (w/w).

The pharmaceutical compositions may also comprise one or more additional therapeutic agents, excipients, or diluents including, but not limited to, absorbents, anti-irritants, preservatives, antioxidants, coloring agents/pigments, emollients (moisturizers), emulsifiers, film-forming/holding agents, prescription drugs, surfactants/detergent cleansing agents, penetration enhancers, viscosity enhancers, and thickeners.

The pharmaceutical compositions of the present application can include those suitable for any acceptable route of administration. Suitable examples of acceptable routes of administration include topical, ocular, topical ocular, injection, and the like.

Compositions and formulations described herein can be conveniently presented in a unit dosage form or a multi-dosage form. In some instances, compositions and formulations described herein can be conveniently presented in a dosage form (unit or multiple) that can be used with or in an automated dosing apparatus or regimen.

In some instances, the topical compositions of the present disclosure can be prepared and used in the form of a non-aqueous solution, a suspension, or an emulsion. In some instances, the topical compositions can be in an emulsion form. In some instances, the topical compositions can be in an organic solution. In some instances, the compositions can be in the form of an organic composition without aqueous components.

In some instances, the topical composition is an ophthalmic composition, e.g., for intraocular administration. Suitable examples of topical ophthalmological compositions described herein are a non-aqueous solution, a suspension, or an emulsion.

In some instances, the compositions described herein can be stable compositions. The compositions can provide chemical stability of the muscarinic cholinergic receptor agonist in the composition for at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, or more, at room temperature.

In some instances, the compositions described herein can be non-irritating. The compositions described herein can, in some instances, cause minimal or no irritation in a mammalian eye.

Any one of these ophthalmic compositions can be administered directly to the eye, using, for example, a plastic or latex applicator (e.g., a single-use applicator), an eye dropper, a glass pipette, or a rubber bulb.

This disclosure also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

In some instances, a kit can comprise a dispensing device preloaded with a composition as described herein.

Also provided herein are methods for treating ocular diseases or conditions using the compositions, and methods described herein. In some instances, the ocular diseases or conditions can be presbyopia.

In some instances, methods are providing for administering, to the eye of a subject, an effective amount of a muscarinic cholinergic receptor agonist. In some instances, the compositions described herein can be in the form of an eye drop. In some instances, the methods can further comprise applying the eye drop to an eye of the subject.

The invention will be further described in the following examples, which do not limit the scope of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1: Droplet Sizes in Atropine Nano-Emulsions

Atropine free base nano-emulsion compositions were prepared. Table 1 below shows oil droplet particle size distribution of MCT/F6H8 nano-emulsions below 10 nm.

TABLE 1
Oil Droplet Particle Size Distribution of CBT-009 Nano-Emulsions

| | |
|---|---|
| | Average oil droplet particle |
| Composition of Atropine | size distribution (nm) |

| Nano-emulsion | D 50 | D 90 |
|---|---|---|
| 3% MCT:97% F6H8 | 2.2 | 3.4 |
| 8% MCT:92% F6H8 | 1.4 | 2.2 |
| 10% MCT:90% F6H8 | 1.7 | 2.5 |
| 20% MCT:80% F6H8 | 2.2 | 3.8 |
| 50% MCT:50% F6H8 | 4.7 | 7.1 |

### Example 2: Systemic Exposure from Atropine Nano-Emulsions

An atropine free base nano-emulsion was analyzed for systemic exposure after ocular administration in rabbits. Table 2 below shows that 0.025% CBT-009 in 10% MCT:90% F6H8 nano-emulsion had lower systemic exposure of atropine as compared to the 0.03% atropine sulfate aqueous solution when both formulations were dosed twice daily for 28 days in rabbits.

TABLE 2
Systemic Exposure of Atropine Following Twice Daily Administration
of 0.02% Atropine Free Base Nano-emulsion and 0.03% Atropine
Sulfate Aqueous Solution in Rabbits for 28 Days

| | Systemic Exposure |
|---|---|
| | AUC/Dose (ng*hr/mL/mg) |
| Dose Formulation | Day 28 ⁽²⁾ |
| 0.025% atropine free base in | 12.1 ± 4.0 |
| 10% MCT:90% F6H8 nano-emulsion | |
| 0.03% atropine sulfate aqueous | 20.9 ± 9.7 |
| solution | |

### Example 3: Ocular Tissue Distribution of Atropine Free Base Nano-Emulsions

Table 3 below shows significant atropine concentrations in rabbit's ocular tissues when 12 μL of 0.0200 atropine free base nano-emulsion was dosed as compared to rabbit's ocular tissue concentrations when 50 CL of atropine sulfate aqueous solution was dosed.

TABLE 3
Atropine Concentrations in Ocular Tissues Following One Drop Administration of 0.02%
Atropine Free Base Nano-emulsion and 1% Atropine Sulfate Aqueous Solution in Rabbits

| | Atropine Concentration in Ocular Tissues | | | | | | |
|---|---|---|---|---|---|---|---|
| | | | | | | Retina- | |
| | | Conjunctiva | Cornea | AH | ICB | Choroid | Sclera |
| Dose Formulation | Parameter | (ng/g) | (ng/g) | (ng/mL) | (ng/g) | (ng/g) | (ng/g) |
| 0.02% atropine | Conc. at 4 hr | 13.1 | 90.0 | 14.4 | 23.7 | 13.8 | 5.16 |
| free base in 10% | Conc./Dose/Eye ⁽³⁾ | 5460 | 37500 | 6000 | 9880 | 5750 | 2150 |
| MCT:90% F6H8 ⁽¹⁾ | | | | | | | |
| 1% atropine | Conc. at 5 hr | 19.3 | ~5 | ~0.6 | ~3 | ~1 | ~3 |
| sulfate aqueous | Conc./Dose/Eye ⁽³⁾ | 38.6 | 10.0 | 1.2 | 6.0 | 2.0 | 6.0 |
| formulation ⁽²⁾ | | | | | | | |

Conc. = concentration; MCT = Medium Chain Triglyceride; F6H8 = Perfluorohexyloctane; AH = Aqueous Humor; ICB = Iris Ciliary Body.
⁽¹⁾ 0.02% atropine free base in 10% MCT:90% F6H8 was dosed 12 μL drop size/eye.
⁽²⁾ 1% atropine sulfate aqueous solution was dosed with 50 μL drop size/eye. Tissue concentrations were estimated from visual graphs (reference article Wang_2019).
⁽³⁾ Concentration/Dose was calculated based on dose concentration and drop size per eye.

### Example 4

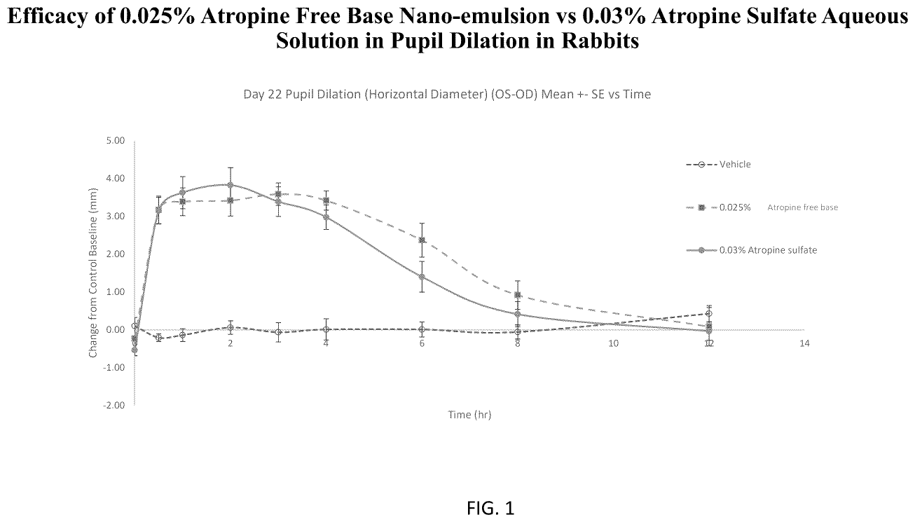

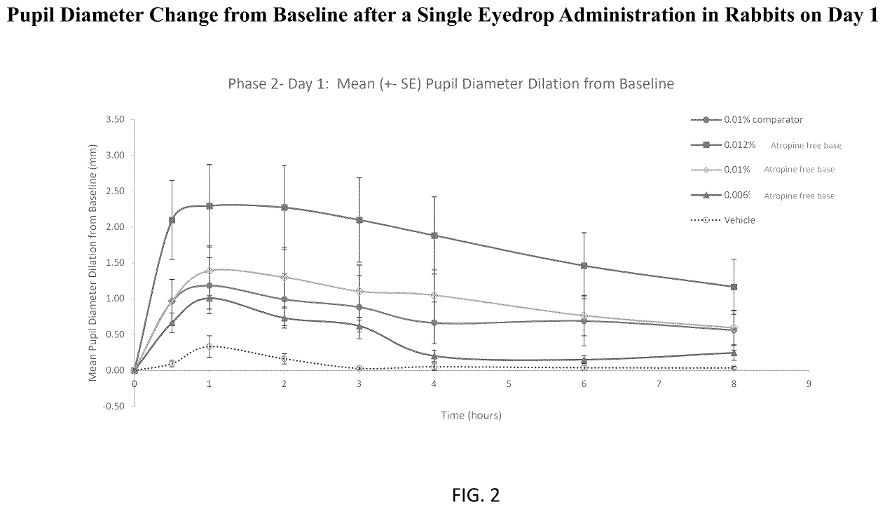

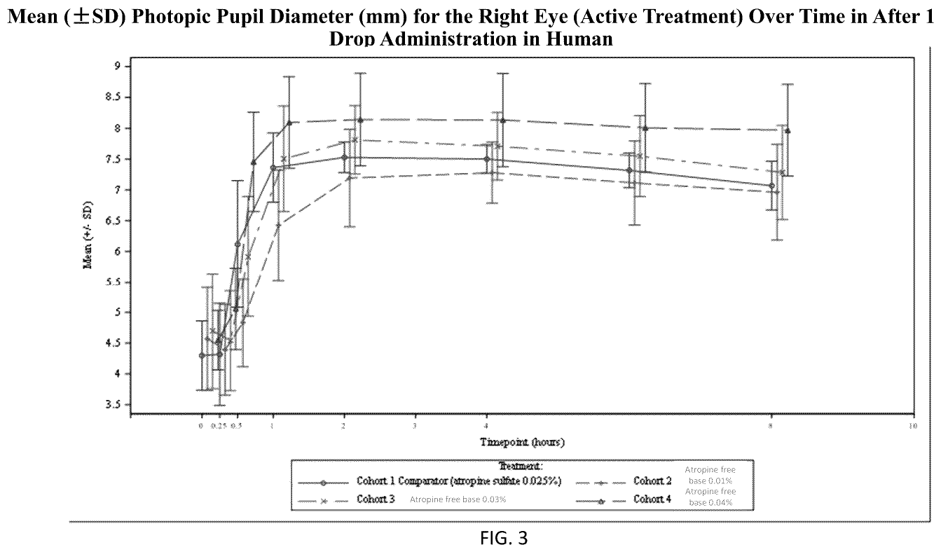

FIG. 1 shows efficacy of atropine in dilating pupil size when atropine free base nano-emulsions and atropine sulfate aqueous solution were dosed twice daily for 22 days. 0.025% atropine free base nano-emulsion shows pro-longed pupil dilation as compared to 0.03% atropine sulfate aqueous solution. FIG. 2 shows that 0.01% and 0.012% atropine free base nano-emulsions had superior efficacy in pupil dilation as compared to 0.01% atropine sulfate aqueous solution after 1 drop administration in rabbits on Day 1. FIG. 3 shows that 0.02% atropine free base nano-emulsion dosed at 12 μL had superior efficacy in pupil dilation as compared to 0.03% atropine sulfate aqueous solution dosed with 40 μL in human.

### Example 5

Atropine free base Nano-emulsions are as safe as atropine sulfate aqueous solutions. Atropine free base nano-emulsions, 0.01, 0.02, and 0.04% have been shown to be safe and well tolerated in rabbits when dosed twice daily for 6 months, as shown in Table 4 below.

0.02% atropine free base Nano-emulsion had been shown safe and well tolerated in human when dosed once daily for 28 days, as shown in Table 5 below. Treatment Emergent Adverse Events (TEAEs) such as vision blurred, glare reported in the clinical trial is believed pharmacologically related to pupil dilation of atropine.

TABLE 4
Summary of Ocular TEAEs Following
One Drop Administration in Human

| | Cohort 1 | | |
|---|---|---|---|
| | Comparator | Cohort 2 | Cohort 3 |
| | (atropine | atropine free | atropine free |
| | sulfate 0.025%) | base 0.01% | base 0.02% |
| System Organ Class | (N = 8) | (N = 8) | (N = 8) |
| Preferred Term | n (%) m | n (%) m | n (%) m |
| | Right Eye (Active Treatment) | | |
| At least one TEAE | 5 (62.5) 7 | 2 (25.0) 3 | 4 (50.0) 5 |
| Eye disorders | 5 (62.5) 7 | 2 (25.0) 3 | 4 (50.0) 5 |
| Vision blurred | 3 (37.5) 3 | 0 | 3 (37.5) 3 |
| Conjunctival hyperemia | 2 (25.0) 2 | 2 (25.0) 2 | 2 (25.0) 2 |
| Glare | 1 (12.5) 1 | 0 | 0 |
| Conjunctival haemorrhage | 0 | 1 (12.5) 1 | 0 |
| Dry eye | 1 (12.5) 1 | 0 | 0 |
| | Left Eye (Vehicle Treatment) | | |
| At least one TEAE | 3 (37.5) 3 | 3 (37.5) 3 | 2 (25.0) 2 |
| Eye disorders | 3 (37.5) 3 | 3 (37.5) 3 | 2 (25.0) 2 |
| Conjunctival hyperemia | 2 (25.0) 2 | 3 (37.5) 3 | 0 |
| Dry eye | 1 (12.5) 1 | 0 | 2 (25.0) 2 |

Abbreviations: m = number of events; MedDRA = Medical Dictionary for Regulatory Activities; TEAE = treatment-emergent adverse event.
Notes:
A TEAE was defined as an adverse event that occurred following the first administration of IP or, if it started prior to the administration of IP, continued afterwards, and worsened in severity.
If a participant had multiple occurrences of a TEAE, the participant was presented only once in the participant count (n) column for a given system organ class and preferred term. Number of event occurrences was presented under the occurrence (m) column.
Events are coded using MedDRA Version 25.0.

TABLE 5
Summary of Ocular TEAEs Following QD Daily Dose of 0.02%
Atropine Free Bse Nano-emulsion for 28 days in Human

| | | Cohort 2 |
|---|---|---|
| | Cohort 1 | atropine free base |
| | Vehicle | 0.02% |
| System Organ Class | (N = 22) | (N = 27) |
| Preferred Term | n (%) m | n (%) m |
| | Right Eye (Treated) | |
| Participants with at least one TEAE | 14 (63.6) 59 | 25 (92.6) 150 |
| Eye disorders | 11 (50.0) 23 | 24 (88.9) 103 |
| Vision blurred | 1 (4.5) 3 | 23 (85.2) 33 |
| Photophobia | 0 | 9 (33.3) 21 |
| Lacrimation increased | 4 (18.2) 8 | 4 (14.8) 16 |
| Conjunctival hyperemia | 1 (4.5) 1 | 4 (14.8) 4 |
| Eye pruritus | 1 (4.5) 2 | 4 (14.8) 8 |
| Dry eye | 2 (9.1) 3 | 1 (3.7) 1 |
| Eye pain | 0 | 4 (14.8) 4 |
| Foreign body sensation in eyes | 1 (4.5) 1 | 3 (11.1) 3 |
| Glare | 0 | 4 (14.8) 5 |
| Eye irritation | 2 (9.1) 2 | 0 |
| General disorders and administration | 6 (27.3) 36 | 6 (22.2) 47 |
| site conditions | | |
| Administration site irritation | 2 (9.1) 13 | 1 (3.7) 1 |
| Administration site pain | 3 (13.6) 19 | 6 (22.2) 33 |
| | Left Eye (Untreated) | |
| Participants with at least one TEAE | 4 (18.2) 6 | 4 (14.8) 5 |
| Eye disorders | 4 (18.2) 6 | 4 (14.8) 5 |
| Dry eye | 2 (9.1) 3 | 1 (3.7) 1 |
| Vision blurred | 0 | 2 (7.4) 2 |

Abbreviations: m = number of events; MedDRA = Medical Dictionary for Regulatory Activities; TEAE = treatment-emergent adverse event.
Notes:
A TEAE was defined as an adverse event that occurred following the first administration of IP or, if it started prior to the administration of IP, continued afterwards, and worsened in severity.
If a participant had multiple occurrences of a TEAE, the participant was presented only once in the participant count (n) column for a given system organ class and preferred term. Number of event occurrences was presented under the occurrence (m) column.
Events are coded using MedDRA Version 25.0.

### Example 6

Atropine free base Nano-emulsions are more stable than atropine aqueous solution. Stability of atropine free base Nano-emulsion shows that atropine is stable in the emulsion system for beyond 1 year, whereas atropine sulfate aqueous solutions tend to have low pH to maintain stability beyond 6 months. However, low pH aqueous solutions tend to cause irritation in the eyes (Berton et al_2020).

TABLE 6
Summary of Ongoing In-Lab Development Stability for Representative Atropine
Free Base Nano-emulsions (10% MCT:90% F6H8) in PET Dropper Bottles

| Atropine free | | | | | | | | |
|---|---|---|---|---|---|---|---|---|
| base Conc. | | | Relative Assay % | | | | | |
| (% w/w) | Container | Storage | Initial | | | | | |
| (Lot#) | Type | Condition | (T₀) | 1 M | 3 M | 6 M | 9 M | 12 M |
| 0.01% | PET | 25° C./40% RH | 100.0 | 101.3 | 102.2 | 103.2 | 100.4 | 102.0 |
| S20220411-07 | | 40° C./25% RH | 100.0 | 101.3 | 101.7 | 100.3 | 100.2 | 101.8 |
| 0.02% | PET | 25° C./40% RH | 100.0 | 101.5 | 100.4 | 100.4 | 100.8 | 100.7 |
| S20220411-08 | | 40° C./25% RH | 100.0 | 100.9 | 102.5 | 101.6 | 100.8 | 99.6 |
| 0.04% | PET | 25° C./40% RH | 100.0 | 101.8 | 100.4 | 102.6 | 101.1 | 100.6 |
| S20220411-09 | | 40° C./25% RH | 100.0 | 100.9 | 99.2 | 101.0 | 99.4 | 102.9 |
| Atropine free | | | Average Oil Droplet Particle Size | | | | | |
| base Conc. | | | Distribution (n.m.) | | | | | |
| (% w/w) | Container | Storage | Initial | | | | | |
| (Lot#) | Type | Condition | (T₀) | 1 M | 3 M | 6 M | 9 M | 12 M |
| 0.01% | PET | 25° C./40% RH | 1.7 | 2.5 | 2.5 | 9.0 | NT | 2.3 |
| S20220411-07 | | 40° C./25% RH | 1.7 | 2.4 | 2.3 | 6.7 | NT | 2.4 |
| 0.02% | PET | 25° C./40% RH | 1.7 | 2.4 | 2.3 | 9.3 | NT | 2.5 |
| S20220411-08 | | 40° C./25% RH | 1.7 | 2.3 | 2.4 | 6.3 | NT | 2.4 |
| 0.04% | PET | 25° C./40% RH | 2.1 | 2.5 | 2.4 | 8.7 | NT | 2.4 |
| S20220411-09 | | 40° C./25% RH | 2.1 | 2.4 | 2.4 | 5.7 | NT | 2.4 |

n.m. = nanometer;
NT = Not Tested;
% RH = % Relative Humidity;
PET = Polyethylene Terephthalate;
M = Month

### Example 7

Atropine free base Nano-emulsions are more stable and compatible with PET container system as shown in Table 6 below. Atropine free base Nano-emulsions are less stable and less compatible with LDPE and PP container systems. Tables 7 below show stability of atropine free base Nano-emulsions in different types of container systems. Without being bound by theory, it is believed the atropine free base Nano-emulsion extracted plastic materials of LDPE and PP thus atropine absorbed into the surface area of the container system as seen in assay dropped in LDPE and PP containers. The LDPE and PP plastic polymer materials were detected in samples of atropine free base nano-emulsions.

TABLE 7
Summary of In-Lab Development Stability for Representative Atropine Free
Base Nano-emulsions (10% MCT:90% F6H8) in LDPE and PP Dropper Bottles

| CBT-009 Conc. | Container | Storage | Initial | | | | |
|---|---|---|---|---|---|---|---|
| (% w/w) | Type | Condition | (T₀) | 1 M | 3 M | 6 M | 12 M |
| 0.01% CBT-009 | LDPE | 25° C./40% RH | 100.0 | 97.4 | 93.4 | 89.6 | NT |
| | | 40° C./25% RH | 100.0 | 92.9 | 84.0 | NT | NT |
| 0.025% CBT-009 | LDPE | 25° C./40% RH | 100.0 | 99.4 | 94.0 | 90.9 | NT |
| | | 40° C./25% RH | 100.0 | 93.8 | 87.3 | NT | NT |
| 0.04% CBT-009 | LDPE | 25° C./40% RH | 100.0 | 96.7 | 96.0 | 90.1 | NT |
| | | 40° C./25% RH | 100.0 | 93.2 | 89.6 | NT | NT |
| 0.01% CBT-009 | PP | 25° C./40% RH | 100.0 | 97.5 | 97.4 | 96.4 | 94.7 |
| | | 40° C./25% RH | 100.0 | 94.7 | 93.5 | 91.4 | 90.3 |
| 0.02% CBT-009 | PP | 25° C./40% RH | 100.0 | 98.3 | 97.3 | 97.0 | 90.6 |
| | | 40° C./25% RH | 100.0 | 97.0 | 95.0 | 91.8 | 86.3 |
| 0.04% CBT-009 | PP | 25° C./40% RH | 100.0 | 98.1 | 98.6 | 96.3 | 93.8 |
| | | 40° C./25% RH | 100.0 | 95.4 | 94.0 | 92.4 | 87.0 |

NT = Not Tested;
% RH = % Relative Humidity;
PP = Polypropylene,
LDPE = Low Density Ethylene;
M = Month

### Example 8

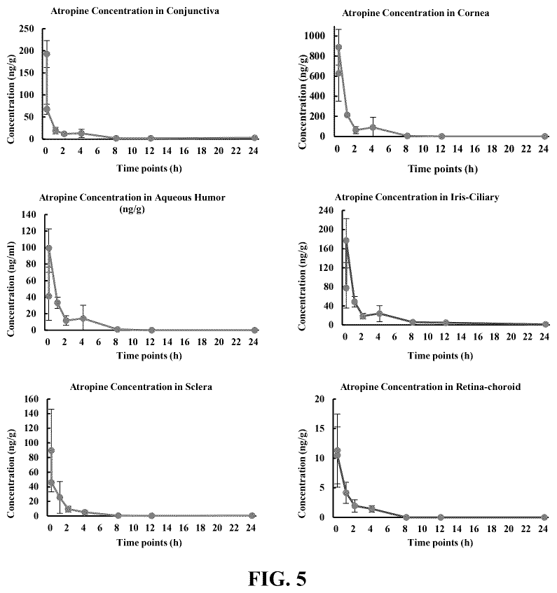

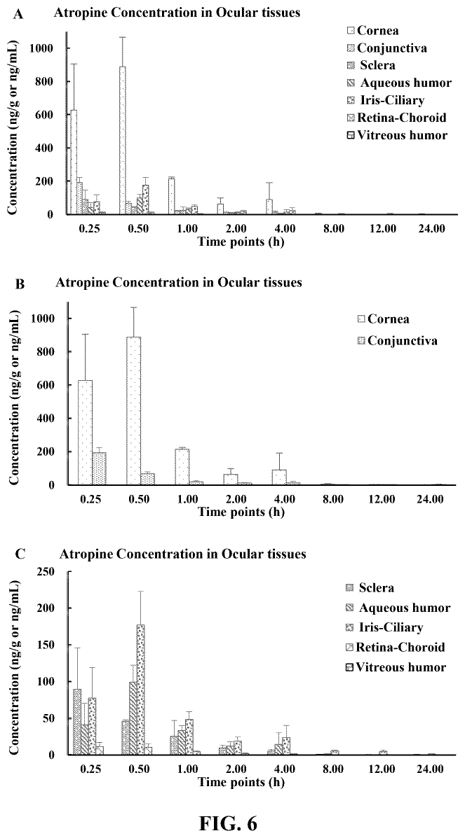

After LC-MS/MS measurement and calculation of the results, it can be seen that the distribution of drug concentration of atropine in ocular tissues at different times after topical ocular administration of 0.02% atropine-10% MCT-F6H8 (0.0043 mg in each eye, which is equivalent to the administration of 40-50 L of atropine sulfate drops at 0.01% in each eye) is as follows in FIG. 5 and FIG. 6 and Table 8. The drug concentrations of atropine in plasma at different times are shown in Table 9 below. Drug concentrations in different parts of ocular tissues showed that the drug was rapidly absorbed in the eye after administration, reached to the concentration peak after 0.25-0.5 h and then gradually decreased, only a small amount of the drug existed in various parts of the ocular tissue after 8 h-12 h. There was almost no drug in the vitreous humor, and only a small amount of the drug was in the retina and the choroid after drug administration. Drug concentrations in plasma showed concentration peak at 0.25 h after administration, followed by rapid metabolism of the drug, reached to the lowest detection limit after 1 h.

TABLE 8
Drug Concentrations in Vitreous Humor at Different
Time After Topical Ocular Dosing of Atropine-10%
MCT-F6H8 to Both Eyes in Rabbits (n = 2)

| Number | C_{0.25 hr (ng/mL)} | C_{0.5 hr (ng/mL)} | C_{1 hr (ng/mL)} | C_{2 hr (ng/mL)} |
|---|---|---|---|---|
| 1-L | 2.04 | BLQ | BLQ | BLQ |
| 1-R | BLQ | BLQ | BLQ | BLQ |
| 2-L | BLQ | BLQ | BLQ | BLQ |
| 2-R | BLQ | BLQ | BLQ | BLQ |
| Number | C_{4 hr (ng/mL)} | C_{8 hr (ng/mL)} | C_{12 hr (ng/mL)} | C_{24 hr (ng/mL)} |
| 1-L | BLQ | BLQ | BLQ | BLQ |
| 1-R | 0.476 | BLQ | BLQ | BLQ |
| 2-L | BLQ | BLQ | BLQ | BLQ |
| 2-R | BLQ | BLQ | BLQ | BLQ |

Abbreviation: BLQ = below limit of quantification (0.04 ng/mL).

TABLE 9
Plasma Concentrations at Different Time After Topical
Ocular Dosing of Atropine-10% MCT-F6H8 to Both
Eyes in Rabbits (n = 2) and PK Parameters

| Number | C_{0.25 hr (ng/mL)} | C_{0.5 hr (ng/mL)} | C_{1 hr (ng/mL)} | C_{2 hr (ng/mL)} |
|---|---|---|---|---|
| 1 | 1.06 | 0.432 | 0.100 | BLQ |
| 2 | 0.738 | 0.432 | 0.073 | BLQ |
| Averages | 0.899 | 0.432 | 0.086 | BLQ |
| Number | C_{4 hr (ng/mL)} | C_{8 hr (ng/mL)} | C_{12 hr (ng/mL)} | C_{24 hr (ng/mL)} |
| 1 | BLQ | BLQ | BLQ | BLQ |
| 2 | BLQ | BLQ | BLQ | BLQ |
| Averages | BLQ | BLQ | BLQ | BLQ |

| | |
|---|---|
| PK Parameters | Cₘₐₓ: 0.899 ng/mL; Tₘₐₓ: 0.3 h; |
| | AUC_{0-24 h}(h*ng/mL): 0.422; |
| | AUC₀₋ₗₐₛₜ(h*ng/mL): 0.379 |

Abbreviation: BLQ = below limit of quantification (0.04 ng/mL); T_{1/2} and AUC_{0-inf} could not be calculated due to lack of sufficient data.

TABLE 10
Pharmacokinetic parameters in Ocular Tissues After Topical Ocular Administration
of Atropine-10% MCT-F6H8 to Both Eyes in Rabbits (n = 4 eyes)

| | Dose | | | | | | |
|---|---|---|---|---|---|---|---|
| | (mg/each | Cₘₐₓ | Tₘₐₓ | T_{1/2} | AUC_{0-24 h} | AUC₀₋ₜₗₐₛₜ | AUC_{0-inf} |
| Ocular Tissues | eye) | (ng/g) | (h) | (h) | (h*ng/g) | (h*ng/g) | (h*ng/g) |
| Cornea | 0.0043 | 888 | 0.5 | NR | 930 | 930 | NR |
| Conjunctiva | 0.0043 | 193 | 0.25 | NR | 173 | 173 | NR |
| Iris/Ciliary | 0.0043 | 177 | 0.5 | 7.3 | 266 | 266 | 279 |
| Body | | | | | | | |
| Aqueous humor | 0.0043 | 99.4 | 0.5 | 1.5 | 123 | 121 | 123 |
| Sclera | 0.0043 | 89.6 | 0.25 | NR | 88.3 | 88.3 | NR |
| Retina/Choroid | 0.0043 | 11.3 | 0.25 | NR | 16.7 | 13.8 | NR |

Note:
NR is not reportable (Rsq < 0.85%); Pharmacokinetic parameters in the vitreous could not be calculated statistically due to lack of data points.

As shown in FIG. 6 and Table 10, ocular tissues reached the drug peak at 0.25-0.5 h after topical administration of 0.02% Atropine-10% MCT-F6H8, and the atropine concentration in different tissues was cornea>conjunctiva>iris-ciliary body>aqueous humor>sclera>retina>vitreous body. Generally, there are two pathways to absorb the drug of topical administration, including transcorneal pathway and conjunctival-scleral pathway. The corneal absorption pathway is following cornea, aqueous humor, iris-ciliary body, and vitreous body, and the conjunctival absorption pathway is following conjunctiva, sclera, and choroid-retina. There was almost no drug in the vitreous body, so it is difficult for atropine to be delivered to the retina via the corneal route through the vitreous body, and a small concentration of the drug was existed in the retina, which is most likely absorbed by the conjunctiva and then delivered to the retina-choroid via the sclera.

Comparing the drug concentration and delivery in ocular tissues after administration atropine sulfate eye drops described in Wang et al., Acta Ophthalmol, 2019(97):238-247 and Ji et al., Pharmaceutical Research, 38(11):1931-1946, it is difficult for atropine sulfate eye drops to be absorbed and transferred to the posterior segment of the eye through the corneal pathway, and the drug is mainly absorbed through the conjunctiva-sclera pathway and transmitted to the choroid-retina via the sclera. This is due to the fact that atropine sulfate has a pKa of 9.43, after contact with tears on the ocular surface, making it difficult to be absorbed through the cornea. The distribution of drug concentration in each ocular tissue at different times after ocular administration of 0.02% atropine-10% MCT-F6H8 showed that atropine-10% MCT-F6H8 was mainly absorbed through the corneal route. This is because the Log P of atropine freebase of is 1.83, which has a good corneal permeability, coupled with the good surface diffusion and tear film stability of F6H8, the drug concentration in the cornea is higher than the drug concentration in the conjunctiva within 8 h after administration.

The mechanism of atropine action in controlling myopia progression is still unclear, and it is mostly believed that there were complex interactions between atropine and multiple receptors on different tissues of the eye, slowing down the deepening of myopic refractive error and axial progression through multiple pathways. According to the above drug distribution in ocular tissues at different times after administration and the results of the above-mentioned efficacy study, it can be seen that atropine was mostly metabolized in the ocular tissues and its effect disappeared after 8 h administration of Atropine-10% MCT-F6H8.

### Example 9: Solubility of Aceclidine in Exemplary Formulations

### 9.1 Experimental

An appropriate amount of aceclidine was dissolved in various MCT (to obtain a mixed solution), then SFA (F6H8 or F4H5) was added to the solution in the indicated proportions and the solution was mixed well with magnetic stirring. The obtained mixture was filtered through 0.22 μm filter and the filtrated solution was analyzed by GC.

### 9.2 Chromatographic Conditions

### Instrument: Agilent-8860(CGC-001)

| Column: | HP-5, 30 m*0.32 mm * 0.5 μm | | |
|---|---|---|---|
| Column | 50° C. | Sample injector | 280° C. |
| temperature: | | temperature: | |
| Detector | 300° C. | Constant current | 2.0 mL/min |
| temperature: | | mode, flow rate: | |
| Injection volume: | 1 μL | Run time: | 45.3 min |
| Split ratio: | 30:1 | Detector: | FID |
| | Rate ° C./ min | Temperature/° C. | Hold/ min |
| TPD | | 50 | 2 |
| | 6 | 280 | 5 |

### 9.3 Results

TABLE 11
Aceclidine solubility (mg/g)

| | Solubility |
|---|---|
| Solvents | RT |

| | | |
|---|---|---|
| MCT | 100% MCT | 320.9(Unsaturated) |
| | 10% MCT/90% F6H8 | 17.0(Unsaturated) |
| Captex 355 | 100% Captex 355 | 203.4(Unsaturated) |
| EP/NF/JPE * | 10% Captex 355/F6H8 | 21.4(Unsaturated), |
| | | 1.5%-2% is stable |
| | | and uniform |
| Captex | 100% Captex 8000 | 164.0(Unsaturated) |
| 8000NF ** | 10% Captex 8000/F6H8 | 17.0(Unsaturated), |
| | | 2% is stable and |
| | | uniform |
| Captex 300 | 100% Captex 300 | 66.7(Unsaturated) |
| EP/NF * | | |

* Captex 355 EP/NF/JPE and Captex 300 EP/NF are each a Glyceryl Tricaprylate/Tricaprate.
** Captex 8000NF is a Glyceryl Tricaprylate, Tricaprylin.

### Example 10: Solubility of Lifitegrast in Exemplary Formulations

### 10.1 Experimental

An appropriate amount of lifitegrast was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 10.2 Chromatographic Conditions

Chromatographic column: Agilent ZORBAX SB-C18, 4.6*250 mm, 5 μm
Mobile phase A: 0.1% Phosphoric Acid-H2O
Mobile phase B: Acetonitrile
Flow rate: 1.0 mL/min Wavelength: UV/220 nm
Injection volume: 10 μL Column temperature: 35° C.

| Time/min | Mobile phase A | Mobile phase B |
|---|---|---|
| 0.01 | 80 | 20 |
| 30.00 | 20 | 80 |
| 30.01 | 80 | 20 |
| 40.00 | 80 | 20 |

Since the auxiliary peaks of MCM and MC8-2 overlapped with the main peaks in the above gradient method, the analytical methods for MCM and MC8-2 were adjusted as follows:
Chromatographic column: Agilent ZORBAX SB-C18, 4.6*250 mm, 5 μm
Mobile phase A: 0.1% Phosphoric Acid-H2O
Mobile phase B: Acetonitrile
Flow rate: 1.0 mL/min Wavelength: UV/220 nm
Injection volume: 10 μL Column temperature: 35° C.

| Time/min | Mobile phase A | Mobile phase B |
|---|---|---|
| 0.01 | 80 | 20 |
| 30.00 | 40 | 60 |
| 30.01 | 80 | 20 |
| 40.00 | 80 | 20 |

### 10.3 Results

TABLE 12
Lifitegrast Saturated solubility (mg/g)

| | | Saturated |
|---|---|---|
| | | Solubility |
| Solvents | | RT |
| MCT | 10% MCT/90% F6H8 | 0 |
| | 20% MCT/80% F6H8 | 0 |
| | 50% MCT/50% F6H8 | 0.004 |
| Captex 355 | 100% Captex 355 | 0.27 |
| EP/NF/JPE* | | |
| Captex 8000NF* | 100% Captex 8000 | 0.56 |
| Captex 300 EP/NF* | 100% Captex 300 | 0.28 |

*Captex 355 EP/NF/JPE and Captex 300 EP/NF are each a Glyceryl Tricaprylate/Tricaprate.
**Captex 8000NF is a Glyceryl Tricaprylate, Tricaprylin.

### Example 11: Solubility of Atropine in Exemplary Formulations

### 11.1 Experimental

An appropriate amount of atropine was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 11.2 Chromatographic Conditions

Chromatographic column: Shimadzu Shim-pack GIST C18-AQ HP 3 μm, 4.6 mm (ID)x 100 mm (L), 3 μm
Guard column: Shimadzu Shim-pack GIST HP (G) C18 3 m, 2.1 mm (ID)x 10 mm (L), 3 μm
Mobile phase A: 0.02% TFA/water
Mobile phase B: 0.02% TFA/acetonitrile
Flow rate: 0.5 mL/min Wavelength: 220 nm
Injection volume: 5 μL Column temperature: 30° C.
Time (min) % Mobile Phase A % Mobile Phase B

| Time (min) | % Mobile Phase A | % Mobile Phase B |
|---|---|---|
| 0 | 100 | 0 |
| 10 | 60 | 40 |
| 20 | 1 | 99 |
| 21 | 100 | 0 |
| 30 | 100 | 0 |

### 11.3 Results

TABLE 13
Atropine Saturated solubility (mg/g)

| | | Saturated | Saturated |
|---|---|---|---|
| | | Solubility | Solubility |
| Solvents | | RT | 2-8° C. |
| MCT | 100% MCT | 7.2 | 6.3 |
| | 10% MCT/90% | 0.7 | 0.22 |
| | F6H8 | | |
| | 10% MCT/90% | 1.6 | 0.54 |
| | F4H5 | | |
| | 20% MCT/80% | 0.7 | 0.29 |
| | F6H8 | | |
| | 20% MCT/80% | 1.7 | 0.63 |
| | F4H5 | | |
| Captex 355 | 100% Captex 355 | 7.9 | 5.6 |
| EP/NF/JPE | 10% Captex | 0.69 | There was no |
| | 355/90% F6H8 | | precipitation |
| | | | at 2-8° C. |
| Captex 8000NF | 100% Captex 8000 | 11.4 | 6.7 |
| | 10% Captex | 0 h: 1.04 | 48 h: 0.27 |
| | 8000/90% F6H8 | 24 h: 0.58 | 5 days: 0.23 |
| Captex 300 EP/NF | 100% Captex 300 | 8.9 | There was no |
| | | | precipitation |
| | | | at 2-8° C. |
| | 10% Captex | 0.8 | 0.35 |
| | 300/90% F6H8 | | |

*Captex 355 EP/NF/JPE and Captex 300 EP/NF are each a Glyceryl Tricaprylate/Tricaprate.
**Captex 8000NF is a Glyceryl Tricaprylate, Tricaprylin.

### Example 12: Solubility of Acoltremon (AR-15512) in Exemplary Formulations

### 12.1 Experimental

An appropriate amount of Acoltremon (AR-15512) was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 12.2 Chromatographic Conditions

Chromatographic column: Shimadau Shim-pack GIST C18 5 um, 4.6 mm (ID)×250 mm (L), 5 μm
Mobile phase A: 0.05% (v/v) TFA/water
Mobile phase B: 0.05% (v/v) TFA/acetonitrile
Flow rate: 1.0 mL/min Wavelength: 214 nm
Injection volume: 10 μL Column temperature: 45° C.
Time (min) % Mobile Phase A % Mobile Phase B

| Time (min) | % Mobile Phase A | % Mobile Phase B |
|---|---|---|
| 0.01 | 60 | 40 |
| 25 | 10 | 90 |
| 25.01 | 60 | 40 |
| 35 | 60 | 40 |

### 12.3 Results

TABLE 14
Acoltremon (AR-15512) Saturated solubility (mg/g)

| | | Saturated |
|---|---|---|
| | | Solubility |
| Solvents | | RT |
| MCT | 100% MCT | 3.12 |
| | 10% MCT/90% F6H8 | 0.29 |
| | 10% MCT/90% F4H5 | 0.28 |
| | 20% MCT/80% F6H8 | 0.55 |
| | 20% MCT/80% F4H5 | 0.55 |
| Captex 170EP | 100% Captex 170 | 2.03 |
| | 10% Captex 17090%/F6H8 | 0.22 |
| | 20% Captex 170/80% F6H8 | 0.43 |
| | 10% Captex 170/90% F4H5 | 0.21 |
| | 20% Captex 170/80% F4H5 | 0.42 |
| Captex 200P | 100% Captex 200P | 3.21 |
| | 10% Captex | 0.36 |
| | 200P/90% F6H8 | |
| | 20% Captex 200P / | 0.53 |
| | 80% F6H8 | |
| | 10% Captex 200P / | 0.36 |
| | 90% F4H5 | |
| | 20% Captex 200P / | 0.66 |
| | 80% F4H5 | |
| F6H8 | 100% F6H8 | 0.055 |
| F4H5 | 100% F4H5 | 0.064 |
| Captex 8000 | 100% Captex 8000 | 1.84 |
| Captex 355 | 10% Captex 355/90% F6H8 | 0.7 |

Captex 170EP is a Coco-Caprylate/Caprate
Captex 200P is a Propylene Glycol Dicaprylocaprate
Captex 8000 is a Glyceryl Tricaprylate, Tricaprylin
Captex 355 is a Glyceryl Tricaprylate/Tricaprate

### Example 13: Solubility of Lotilaner in Exemplary Formulations

### 13.1 Experimental

An appropriate amount of Lotilaner was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 13.2 Chromatographic Conditions

Chromatographic column: SHIMADAU Shim-pack GIST C18 5 um, 4.6 mm (ID)×250 mm (L), 5 μm
Mobile phase A: 0.05% (v/v) TFA in water
Mobile phase B: 0.05% (v/v) TFA in acetonitrile
Flow rate: 1.0 mL/min Wavelength: 214 nm
Injection volume: 10 μL Column temperature: 45° C.
Time (min) % Mobile Phase A % Mobile Phase B

| Time (min) | % Mobile Phase A | % Mobile Phase B |
|---|---|---|
| 0.01 | 60 | 40 |
| 25 | 10 | 90 |
| 25.01 | 60 | 40 |
| 35 | 60 | 40 |

### 13.3 Results

TABLE 15
Lotilaner solubility (mg/g)

| | | Solubility |
|---|---|---|
| Solvents | | RT |
| MCT | 100% MCT (Unsaturated) | 25.47 |
| | 10% MCT/90% F6H8 | 2.7 |
| | 20% MCT/80% F6H8 | 5.42 |
| Captex 8000NF | 100% Captex 8000 (Unsaturated) | 29.04 |
| | 10% Captex 8000/90% F6H8 | 3.47 |
| Captex 300 EP/NF | 100% Captex 300 (Unsaturated) | 11.32 |
| | 10% Captex 300/90% F6H8 | 1.21 |

### Example 14: Solubility of Brimonidine in Exemplary Formulations

### 14.1 Experimental

An appropriate amount of Brimonidine was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 14.2 Chromatographic Conditions

Chromatographic column: SHIMADZU Shim-pack GIST C18 5 um, 4.6 mm×250 mm, 5 μm
Mobile phase A: 0.05% TFA-H2O
Mobile phase B: 0.05% TFA-Acetonitrile
Flow rate: 1.0 mL/min Wavelength: UV/214 nm
Injection volume: 5 μL Column temperature: 45° C.

| Time/min | Mobile phase A | Mobile phase B |
|---|---|---|
| 0.01 | 95 | 5 |
| 10 | 85 | 15 |
| 15 | 85 | 15 |
| 15.01 | 95 | 5 |
| 25 | 95 | 5 |

### 14.3 Results

TABLE 16
Brimonidine Saturated solubility (mg/g)

| | | Saturated |
|---|---|---|
| | | Solubility |
| Solvents | | RT |
| MCT | 10% MCT/90% F6H8 | 0 |
| | 10% MCT/90% F4H5 | 0 |
| | 20% MCT/80% F6H8 | 0.002 |
| | 20% MCT/80% F4H5 | 0.002 |
| Captex 355 | 100% Captex 355 | 0.04 |
| EP/NF/JPE | | |
| Captex 8000NF | 100% Captex 8000 | 0.05 |
| Captex 300 EP/NF | 100% Captex 300 | 0.1 |

### Example 15: Solubility of Cyclosporine in Exemplary Formulations

### 15.1 Experimental

An appropriate amount of Cyclosporine was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 15.2 Chromatographic Conditions

Chromatographic column: SHIMADZU Shim-pack GIST C18 5 μm, 4.6 mm×250 mm, 5 μm
Mobile phase A: 0.05% (v/v) TFA in water
Mobile phase B: 0.05% (v/v) TFA in Acetonitrile
Flow rate: 1.0 mL/min Wavelength: 214 nm
Injection volume: 10 μL Column temperature: 45° C.
Time (min) % Mobile Phase A % Mobile Phase B

| Time (min) | % Mobile Phase A | % Mobile Phase B |
|---|---|---|
| 0.01 | 60 | 40 |
| 25 | 10 | 90 |
| 25.01 | 60 | 40 |
| 35 | 60 | 40 |

### 15.3 Results

TABLE 17
Cyclosporine solubility (mg/g)

| | | Solubility | Clear or |
|---|---|---|---|
| Solvents | | RT | Stratification |
| MCT | 100% MCT (Not | 53.28 | N/A |
| | Saturated) | | |
| | 10% MCT/90% F6H8 | 2.65 | Clear |
| | 20% MCT/80% F6H8 | 8.98 | Clear |
| Captex 8000NF | 100% Captex 8000 | 47.35 | N/A |
| | 10% Captex | 3.13 | Clear |
| | 8000/90% F6H8 | | |
| Captex 300 | 100% Captex 300 | 28.28 | N/A |
| EP/NF | 10% Captex | 2.7 | Clear |
| | 300/90% F6H8 | | |

### Example 16: Solubility of Ivermectin (CBT-273) in Exemplary Formulations

### 16.1 Experimental

An appropriate amount of Ivermectin (CBT-273) was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 16.2 Chromatographic Conditions

Chromatographic column: SHIMADAU Shim-pack GIST C18 5 μm, 4.6 (ID) mm×250 mm (L), 5 μm
Mobile phase: Acetonitrile: Methanol: Water=55:35:10 (v:v:v)
Flow rate: 1.0 mL/min Wavelength: 214 nm
Injection volume: 10 μL Column temperature: 45° C.

### 16.3 Results

TABLE 18
Ivermectin (CBT-273) Saturated solubility (mg/g)

| | | Saturated |
|---|---|---|
| | | Solubility |
| Solvents | | RT |
| F6H8 | 100% F6H8 | 0.04 |
| F4H5 | 100% F4H5 | 0.12 |
| Captex | 100% Captex 8000 | 34.62 |
| 8000 | 10% Captex 8000/90% F6H8 | 1.36 |
| Captex 355 | 100% Captex 355 | 26.51 |
| | 10% Captex 355/90% F6H8 | 0.46 |
| | 20% Captex 355/80% F6H8 | 3.87 |
| | 10% Captex 355/90% F4H5 | 0.61 |
| | 20% Captex 355/80% F4H5 | 3.03 |
| Captex 300 | 100% Captex ® 300 | 27.91 |
| | 10% Captex 300/90% F6H8 | 1.35 |

### Example 17: Dissolution of Aceclidine in F6H8

Methods: Formulations of aceclidine free base were investigated according to the following procedure:

### 1. Determining the Solubility of Aceclidine in F6H8

5 mg aceclidine was added into a PP tube, 1 ml of F6H8 was added, the PP tube was shaken for about 5 minutes and aceclidine was completely dissolved in the solution. Further 18 mg aceclidine was added, and shaking was continued until no insoluble substance was found. The PP tube was centrifuged for 3 minutes, a small amount of yellowish brown oil droplets were observed to be floating. The phenomenon disappeared after re-shaking.

### 2. Preparing GC Samples

The above formulations were centrifuged and the supernatants filtered through 0.45 micron filters without further dilution. One sample was prepared from each solvent for GC analysis.

### 3. Analyzing the GC Samples

The samples were analyzed using a Agilent 8890 GC method with an HP-5 30 m*0.32 mm*0.5 um GC column (150 mm×2.1 mm FD.). The GC protocols and parameters are listed below:

### Parameters:

Gas Flow rate: 2 ml/min

Injection Volume: 0.5 ul (Direct injection)

Column Temp: 70° C.

Inject Temp: 280° C.

Detector Temp (FID): 300° C.

Gradient: 70° C. (hold 2 min)→12° C./min→280° C. (hold 7 min)

Gas Ratio: 30:1

N2: 25 ml/min

H2: 30 ml/min

Air: 300 ml/min

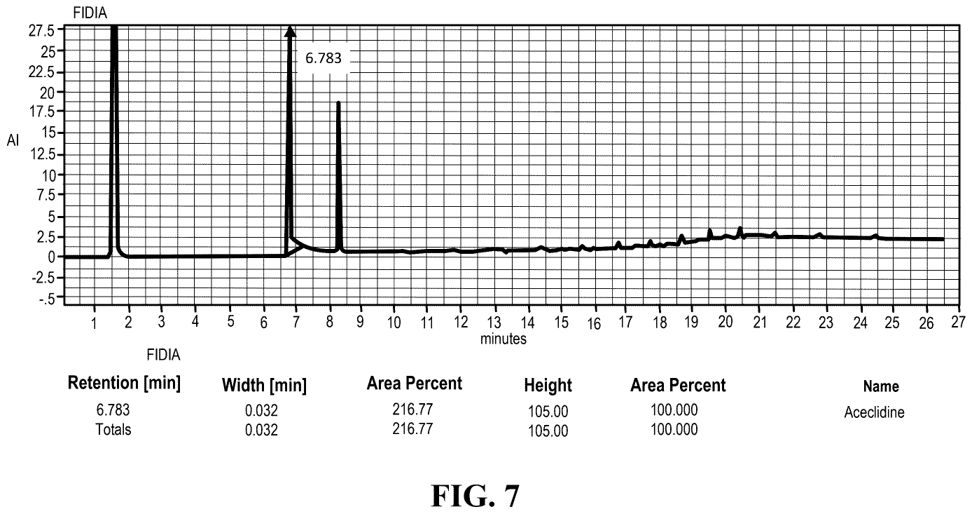

The aceclidine peak is at retention time 6.783 minutes as shown in the chromatograph in FIG. 7.

### Results

The solubility of aceclidine in F6H8 based on the GC method described above was determined to be around 1.5% (w/v).

### Example 18: Solubility of Aceclidine in MCT

### Sample Preparation

20 mg aceclidine was added into a PP tube, and 1 ml MCT was added. The PP tube was shaken for about 5 minutes and the resulting solution became clear. Further 40 mg of aceclidine was added. The PP tube was shaken again. A small amount of transparent oil droplets were found to be insoluble. After centrifugation for 3 minutes, a small amount of yellowish-brown oil droplets were observed to be on floating. The phenomenon disappeared after re-shaking. A small sample was taken and solubility was determined according to the GC method described in Example 17.

The solubility of aceclidine in MCT based on the GC method described in Example 17 was determined to be around 5% (w/v).

### Example 19: Solubility of Aceclidine in 10% MCT in F6H8

Sample preparation was prepared and analyzed based on the protocols described in Examples 17 and 18. The solubility of aceclidine in 10% MCT in F6H8 was determined to be around 2% (w/v).

### Example 20: Stability of a Formulation, Aceclidine in F6H8

### Methods

Aceclidine is dissolved in F6H8 as described in Example 17. The level of aceclidine is measured by the GC method at 25° C. at 1, 3, 6, 9 and 12 months. The aceclidine in the formulation is defined as stable if the level is maintained between 90-110% of the original level.

### Results

During the study period, aceclidine remains stable.

### Example 21: In Vivo Pharmacological Potency in a Rabbit Model

### Method

Aceclidine ophthalmic formulation: 2% aceclidine in F6H8 formulation with MCT as a cosolvent (ratio: MCT:F6H8 1:9)

Animals: Dutch Belted Rabbit

3 female rabbits were given 1 drop of 2% aceclidine once to both eyes on Day 1. Pupil diameter was measured in both eyes of all animals before dosing initiation. The pupil diameter of both eyes of all animals was measured at baseline, 0.5 h, 1 h, 2 h, 4 h, 6 h, 8 h, 12 h and 24 h after dosing.

### Results

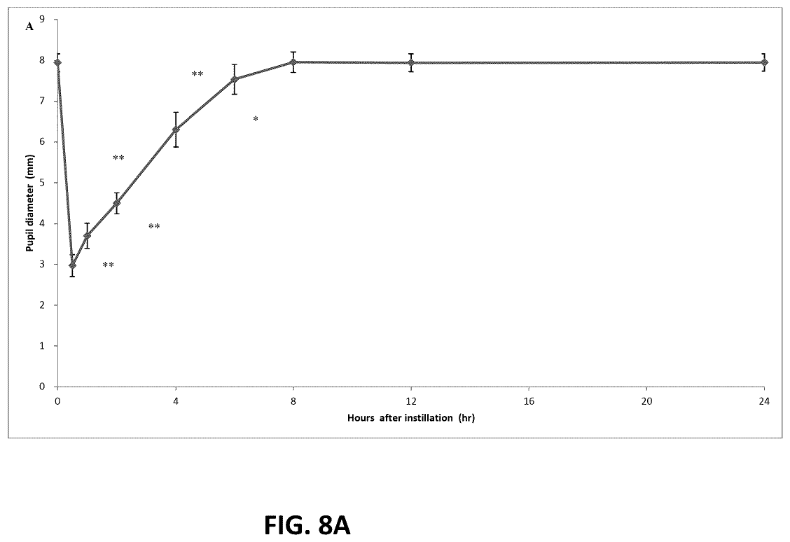

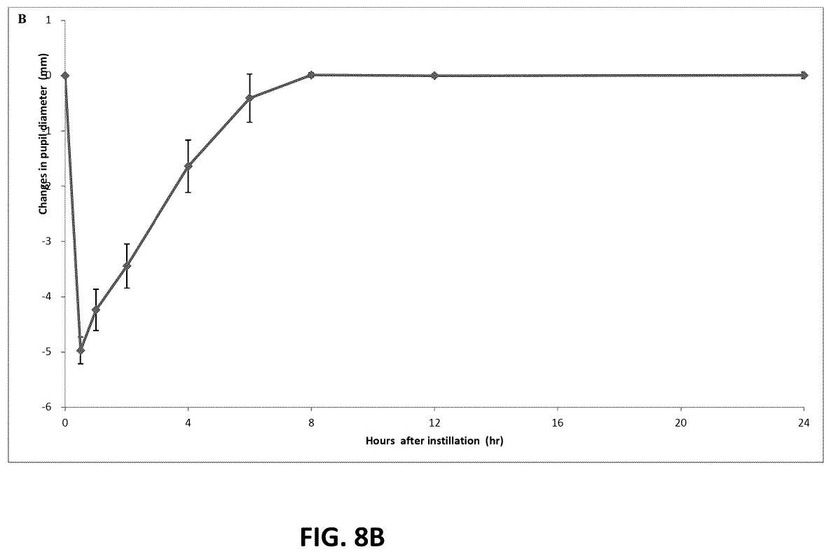

The baseline pupil diameter was 7.94±0.22 mm. 2% aceclidine caused significant pupil contraction from 0.5 hour after instillation (Tab.19, FIG. 8). Compared with the pupil diameter at baseline, significant pupil contraction was noted after the 2% aceclidine ophthalmic formulation treatment prepared as above at the time points of 0.5 h, 1 h, 2 h, 4 h, 6 h and 8 h post dosing (p≤0.05 or 0.01) with pupil diameter changes of −4.97, −4.24, −3.44, −1.64 and −0.40 mm. The peak effect (Emax) of pupil diameter contraction with −4.97 mm occurred at 0.5 hour post-dose.

TABLE 19
Changes in pupil diameter after a single instillation administration
of 2% aceclidine F8H8 MCT (ratio 9:1) formulation
Pupil diameter or changes (mm)

| | Hours (hr) | | | | | | | | |
|---|---|---|---|---|---|---|---|---|---|
| | 0 | 0.5 | 1 | 2 | 4 | 6 | 8 | 12 | 24 |
| | N (Eye) | | | | | | | | |
| | 6 | 6 | 6 | 6 | 6 | 6 | 6 | 6 | 6 |
| Mean | 7.94 | 2.97** | 3.70** | 4.50** | 6.30** | 7.54* | 7.96 | 7.94 | 7.95 |
| Pupil diameter | | | | | | | | | |
| SD | 0.22 | 0.27 | 0.31 | 0.26 | 0.42 | 0.36 | 0.25 | 0.22 | 0.21 |
| Mean- | 0.00 | −4.97 | −4.24 | −3.44 | −1.64 | −0.40 | 0.01 | 0.002 | 0.01 |
| Changes | | | | | | | | | |
| SD | 0.00 | 0.24 | 0.37 | 0.40 | 0.47 | 0.44 | 0.04 | 0.04 | 0.06 |

For pupil diameter,
*P < 0.05 or
**P < 0.01 compared with baseline using the T-TEST.
No statistical comparison was made for pupil diameter changes.

### Example 22: In Vivo Pharmacological Potency in a Rabbit Model

The Effects of Aceclidine on Pupil Diameter after One-Time Topical Instillation

### Method

Test Article: 0.5% aceclidine, 1% aceclidine, 2% aceclidine in F6H8 ophthalmic formulation

Animals: Dutch Belted Rabbits

3 female rabbits per group were given 1 drop of 0.5% aceclidine, 1% aceclidine or 2% aceclidine, respectively, in F6H8, once to both eyes. Pupil diameter was measured in both eyes of all animals before dosing initiation as the baseline. The pupil diameter of both eyes of all animals was measured at 0.5 h, 1 h, 2 h, 4 h, 6 h, 8 h, 12 h and 24 h after dosing to evaluate the effect of aceclidine on pupil diameter.

### Results

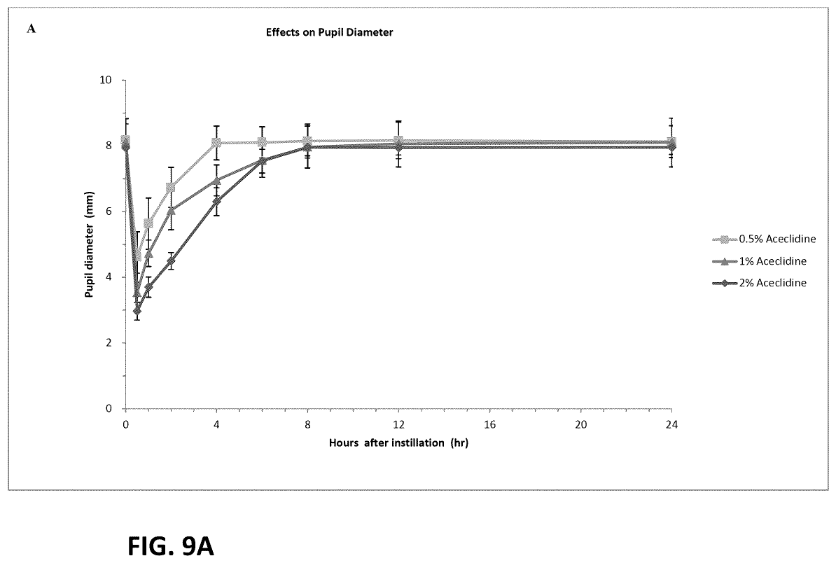

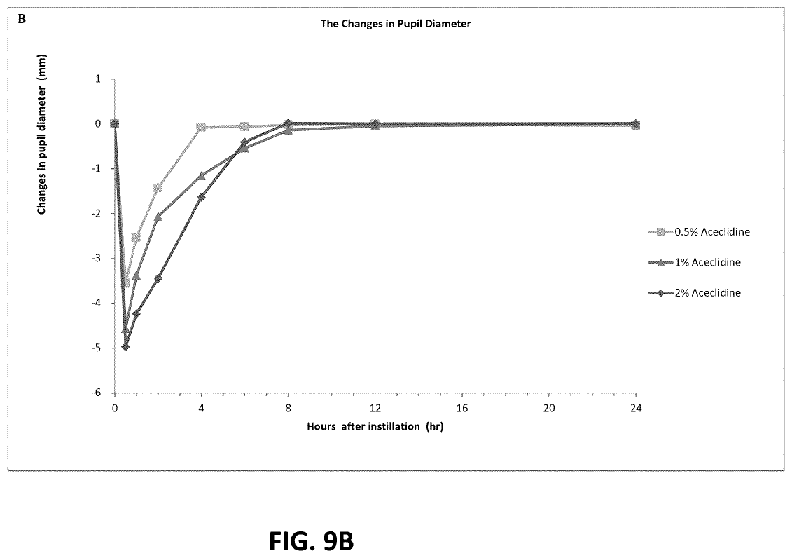

The baseline pupil diameter was 8.17±0.50, 8.11±0.72, and 7.94±0.22 mm at 0.5% aceclidine, 1% aceclidine and 2% aceclidine dose levels, respectively. All three dose levels of aceclidine caused significant pupil contraction from 0.5 hour after instillation and lasted to 2-6 hours (Table 20, FIG. 9). Compared with the pupil diameter at baseline, significant pupil contraction (p≤0.01) was noted after 0.5% aceclidine administration of 0.5 h, 1 h, 2 h with changes in pupil diameter of −3.56, −2.54, and −1.43 mm. Compared with the pupil diameter at baseline, significant pupil contraction (p≤0.01 or 0.05) was noted after 1% aceclidine administration of 0.5 h, 1 h, 2 h and 4 h with pupil diameter changes of −4.57, −3.38, −2.07 and −1.16 mm. Compared with the pupil diameter at baseline, significant pupil contraction (p≤0.01 or 0.05) was noted after the 2% aceclidine administration of 0.5 h, 1 h, 2 h, 4 h, and 6 h with pupil diameter changes of −4.97, −4.24, −3.44, −1.64 and −0.40 mm. The peak effect (Emax) of pupil diameter contraction with −3.56, −4.57 and −4.97 mm occurred at 0.5 hour post-dose at 0.5% aceclidine, 1% aceclidine and 2% aceclidine dose levels, respectively. A dose-effect and relationship of pupillary constriction was observed at 0.5-2% aceclidine dose levels.

TABLE 20
Effects on pupil diameter after a single instillation and administration of aceclidine.

| | Hours (hr) | | | | | | | | |
|---|---|---|---|---|---|---|---|---|---|
| | 0 | 0.5 | 1 | 2 | 4 | 6 | 8 | 12 | 24 |
| | Pupil Diameter (Mean ± SD, mm), N(Eye) = 6 | | | | | | | | |
| 0.5% Aceclidine | 8.17 ± | 4.62 ± | 5.63 ± | 6.74 ± | 8.09 ± | 8.11 ± | 8.15 ± | 8.17 ± | 8.13 ± |
| | 0.50 | 0.77** | 0.78** | 0.61** | 0.51 | 0.48 | 0.52 | 0.55 | 0.49 |
| 1% Aceclidine | 8.11 ± | 3.54 ± | 4.73 ± | 6.04 ± | 6.95 ± | 7.57 ± | 7.97 ± | 8.06 ± | 8.10 ± |
| | 0.72 | 0.59** | 0.40** | 0.59** | 0.47* | 0.52 | 0.63 | 0.70 | 0.74 |
| 2% Aceclidine | 7.94 ± | 2.97 ± | 3.70 ± | 4.50 ± | 6.30 ± | 7.54 ± | 7.96 ± | 7.94 ± | 7.95 ± |
| | 0.22 | 0.27** | 0.31** | 0.26** | 0.42** | 0.36* | 0.25 | 0.22 | 0.21 |
| | Changes in Pupil Diameter (Mean ± SD, mm), N(Eye) = 6 | | | | | | | | |
| 0.5% Aceclidine | 0.00 ± | −3.56 ± | −2.54 ± | −1.43 ± | −0.08 ± | −0.06 ± | −0.02 ± | −0.004 ± | −0.04 ± |
| | 0.00 | 0.65 | 0.67 | 0.40 | 0.05 | 0.07 | 0.02 | 0.08 | 0.06 |
| 1% Aceclidine | 0.00 ± | −4.57 ± | −3.38 ± | −2.07 ± | −1.16 ± | −0.54 ± | −0.14 ± | −0.05 ± | −0.004 ± |
| | 0.00 | 0.80 | 0.74 | 1.05 | 0.75 | 0.33 | 0.14 | 0.05 | 0.04 |
| 2% Aceclidine | 0.00 ± | −4.97 ± | −4.24 ± | −3.44 ± | −1.64 ± | −0.40 ± | 0.01 ± | 0.002 ± | 0.01 ± |
| | 0.00 | 0.24 | 0.37 | 0.40 | 0.47 | 0.44 | 0.04 | 0.04 | 0.06 |

For pupil diameter,
*P < 0.05 or
**P < 0.01 compared with baseline using the T-TEST.
No statistical comparison was made for pupil diameter change

Examples 17, 18 and 19 demonstrate that stable aceclidine formulations can be achieved in F6H8, including in some instances with the addition of a co-solvent. Example 21 shows that equivalent doses of the aceclidine free base F6H8 organic formulation achieved similar efficacy to that of the aceclidine aqueous formulation in a rabbit model. The studies indicate that the muscarinic cholinergic receptor agonist, in a formulation with semifluorinated alkane compound such as F6H8 as liquid vehicle can also be efficacious.

### Example 23: Solubility of Axitinib in Exemplary Formulations

### 23.1 Experimental

An appropriate amount of Axitinib was dissolved in various MCT by vortexing, then SFA (F6H8 or F4H5) was added to the mixture in the indicated proportions and the solution was vortexed. The obtained mixture was filtered through 0.22 μm filter and the filtrated solution was analyzed by GC.

### 23.2 Chromatographic Conditions

Chromatographic column: SHIMADZU Shim-pack GIST C18 5 μm, 4.6 mm×150 mm, 5 μm

Mobile phase A: 0.05% TFA-H2O

Mobile phase B: 0.05% TFA-Acetonitrile

Flow rate: 1.0 mL/min Wavelength: 362 nm

Injection volume: 30 μL Column temperature: 40° C.

TABLE 21

| Time/min | Mobile phase A | Mobile phase B |
|---|---|---|
| 0.00 | 80 | 20 |
| 6.00 | 50 | 50 |
| 6.10 | 80 | 20 |
| 10.00 | 80 | 20 |

### 23.3 Results

TABLE 22
Axitinib Saturated solubility (mg/g)

| | | Saturated |
|---|---|---|
| | | Solubility |
| Solvents | | RT |
| Captex 355 EP/NF/JPE | 100% Captex 355 | 0.03 |
| Captex 8000NF | 100% Captex 8000 | 0.1 |
| Captex 300 EP/NF | 100% Captex 300 | 0.022 |

### EMBODIMENTS

1. A topical ophthalmological composition comprising: about 0.001% to about 5% (w/w) or about 0.0001% to about 20% (w/w) active pharmaceutical ingredient (API), wherein the API has a solubility in medium chain triglyceride (MCT) of at least from about 0.01% to about 10% (w/w), and wherein the API is not atropine or a salt thereof; from about 1% to about 50% (w/w) of an MCT; and from about 50% to about 99% (w/w) of a semi-fluorinated alkane compound.
2. The topical ophthalmological composition of embodiment 1, wherein the composition is non-aqueous.
3. The topical ophthalmological composition of any one of embodiments 1-2, wherein the semi-fluorinated alkane compound is selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).
4. The topical ophthalmological composition of any one of embodiments 1-3, wherein the MCT is present in a concentration of from about 10% to about 50% (w/w) and the semi-fluorinated alkane compound is present in a concentration of from about 50% to about 90% (w/w).
5. The topical ophthalmological composition of any one of embodiments 1-4, wherein the MCT is present in a concentration of from about 10% to about 30% (w/w) and the semi-fluorinated alkane compound is present in a concentration of from about 70% to about 90% (w/w).
6. The topical ophthalmological composition of any one of embodiments 1-5, wherein the API is present in a concentration of from about 0.01% to 1% (w/w).
7. The topical ophthalmological composition of any one of embodiments 1-6, wherein the API is in a free base form.
8. The topical ophthalmological composition of any one of embodiments 1-7, wherein the MCT is a triglyceride of fatty acids and the fatty acids comprise hexanoic acid, octanoic acid, decanoic acid, dodecanoic acid or combinations of two or more thereof.
9. The topical ophthalmological composition of any one of embodiments 1-8, wherein the MCT is present in a concentration of about 5% (w/w) and the semi-fluorinated alkane compound is present in a concentration of about 95% (w/w), wherein the MCT is present in a concentration of about 10% (w/w) and the semi-fluorinated alkane compound is present in a concentration of about 90% (w/w), wherein the MCT is present in a concentration of about 15% (w/w) and the semi-fluorinated alkane compound is present in a concentration of about 85% (w/w), or wherein the MCT is present in a concentration of about 20% (w/w) and the semi-fluorinated alkane compound is present in a concentration of about 80% (w/w).
10. The topical composition of any one of embodiments 1-9, wherein the semi-fluorinated alkane is perfluorohexyloctane (F6H8).
11. The topical composition of any one of embodiments 1-10, wherein the MCT is a triglyceride of fatty acids and the fatty acids consist essentially of hexanoic acid, octanoic acid, decanoic acid, dodecanoic acid or combinations thereof.
12. The topical ophthalmological composition of any one of embodiments 1-11, wherein the topical ophthalmological composition is an emulsion.
13. The topical ophthalmological composition of any one of embodiments 1-12, wherein the API in the topical ophthalmological composition is chemically stable for at least 6 months, at least 1 year, at least 18 months, at least 2 years or longer.
14. The topical ophthalmological composition of any one of embodiments 1-13, wherein the API is not a muscarinic receptor antagonist.
15. The topical ophthalmological composition of any one of embodiments 1-14, wherein the API is selected from alpha agonists, antibiotics, corticosteroids, antinicotinic agents, antiglaucoma agents, antihistamines, antivirals, anti-parasitics, pressure regulators, topical transient receptor potential melastatin 8 (TRPM8) agonists, inhibitors of inflammatory cell binding, cycloplegics/mydriatics, mast cell stabilizers, miotics, ophthalmic NSAIDs, cyclosporine, riboflavin 5′-phosphate ophthalmic compounds, and VEGF inhibitors and VEGF receptor inhibitors.
16. A topical ophthalmological composition consisting essentially of: about 0.001% to about 5% (w/w) or about 0.0001% to about 10% (w/w) active pharmaceutical ingredient (API), wherein the API has a solubility in MCT of at least from about 0.01% to about 10% (w/w), and wherein the API is not atropine or a salt thereof, from about 1% to about 50% (w/w) of a medium chain triglyceride (MCT); and from about 50% to about 99% (w/w) of a semi-fluorinated alkane compound.
17. The topical ophthalmological composition of embodiment 16, wherein the semi-fluorinated alkane compound is selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).
18. The topical ophthalmological composition of any one of embodiments 16 or 17, wherein the MCT is present in a concentration of about 5% (w/w) and the semi-fluorinated alkane compound is present in a concentration of about 95% (w/w), wherein the MCT is present in a concentration of about 10% (w/w) and the semi-fluorinated alkane compound is present in a concentration of about 90% (w/w), wherein the MCT is present in a concentration of about 15% (w/w) and the semi-fluorinated alkane compound is present in a concentration of about 85% (w/w), or wherein the MCT is present in a concentration of about 20% (w/w) and the semi-fluorinated alkane compound is present in a concentration of about 80% (w/w).
19. A topical ophthalmological composition comprising: about 0.01% to about 1% (w/w) or about 0.0001% to about 10% (w/w) active pharmaceutical ingredient (API), wherein the API has a solubility in MCT of at least from about 0.01% to about 10% (w/w), and wherein the API is not atropine or a salt thereof, about 10% to about 30% (w/w) medium chain triglyceride (MCT); and about 70% to about 90% (w/w) semi-fluorinated alkane compound selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).
20. A topical ophthalmological nano-emulsion comprising: about 0.001% to about 5% (w/w) or about 0.0001% to about 20% (w/w) active pharmaceutical ingredient (API), wherein the API has a solubility in MCT of at least from about 0.01% to about 10% (w/w); from about 1% to about 50% (w/w) of a medium chain triglyceride (MCT); and from about 50% to about 99% (w/w) of a semi-fluorinated alkane compound, wherein the nano-emulsion has a droplet particle size D90 of less than about 100 nm.
21. The topical ophthalmological nano-emulsion of embodiment 20, wherein the nano-emulsion has a droplet particle size D90 of less than about 20 nm, less than about 10 nm, or less than about 5 nm, or has a droplet particle size D90 of from about 2 to about 3 nm.
22. The topical ophthalmological nano-emulsion of any one of embodiments 20-21, wherein the nano-emulsion has a peak droplet particle size of less than about 10 nm or has a peak droplet particle size of from about 1 nm to about 5 nm.
23. The topical ophthalmological nano-emulsion of any one of embodiments 20-22, wherein the composition is non-aqueous.
24. The topical ophthalmological nano-emulsion of any one of embodiments 20-23, wherein the semi-fluorinated alkane compound is selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).
25. The topical ophthalmological nano-emulsion of any one of embodiments 20-24, wherein the MCT is present in a concentration of from about 10% to about 50% (w/w) and the semi-fluorinated alkane compound is present in a concentration of from about 50% to about 90% (w/w).
26. The topical ophthalmological nano-emulsion of any one of embodiments 20-25, wherein the MCT is present in a concentration of from about 10% to about 30% (w/w) and the semi-fluorinated alkane compound is present in a concentration of from about 70% to about 90% (w/w).
27. The topical ophthalmological nano-emulsion of any one of embodiments 20-26, wherein the API is present in a concentration of from about 0.01% to 1% (w/w) or from about 0.0001% to about 10% (w/w).
28. The topical ophthalmological nano-emulsion of any one of embodiments 20-27, wherein the API is in a free base form.
29. The topical ophthalmological nano-emulsion of any one of embodiments 20-28, wherein the MCT is a triglyceride of fatty acids and the fatty acids comprise hexanoic acid, octanoic acid, decanoic acid, dodecanoic acid or combinations of two or more thereof.
30. The topical ophthalmological nano-emulsion of any one of embodiments 20-29, wherein the MCT is present in a concentration of about 5% (w/w) and the semi-fluorinated alkane compound is present in a concentration of about 95% (w/w), wherein the MCT is present in a concentration of about 10% (w/w) and the semi-fluorinated alkane compound is present in a concentration of about 90% (w/w), wherein the MCT is present in a concentration of about 15% (w/w) and the semi-fluorinated alkane compound is present in a concentration of about 85% (w/w), or wherein the MCT is present in a concentration of about 20% (w/w) and the semi-fluorinated alkane compound is present in a concentration of about 80% (w/w).
31. The topical ophthalmological nano-emulsion of any one of embodiments 20-30, wherein the semi-fluorinated alkane is perfluorohexyloctane (F6H8).
32. The topical ophthalmological nano-emulsion of any one of embodiments 20-31, wherein the MCT is a triglyceride of fatty acids and the fatty acids consist essentially of hexanoic acid, octanoic acid, decanoic acid, dodecanoic acid or combinations thereof.
33. The topical ophthalmological nano-emulsion of any one of embodiments 20-32, wherein the topical ophthalmological nano-emulsion is an emulsion.
34. The topical ophthalmological nano-emulsion of any one of embodiments 20-33, wherein the API in the topical ophthalmological nano-emulsion is chemically stable for at least 6 months, at least 1 year, at least 18 months, at least 2 years or longer.
35. The topical ophthalmological nano-emulsion of any one of embodiments 20-34, wherein the API is selected from alpha agonists, antibiotics, corticosteroids, anticholinergic agents, antiglaucoma agents, antihistamines, antivirals, anti-parasitics, pressure regulators, topical transient receptor potential melastatin 8 (TRPM8) agonists, inhibitors of inflammatory cell binding, cycloplegics/mydriatics, mast cell stabilizers, miotics, ophthalmic NSAIDs, cyclosporine, riboflavin 5′-phosphate ophthalmic compounds, and VEGF inhibitors and VEGF receptor inhibitors.
36. A topical ophthalmological nano-emulsion consisting essentially of: about 0.001% to about 5% (w/w) or about 0.0001% to about 10% (w/w) active pharmaceutical ingredient (API), wherein the API has a solubility in MCT of at least from about 0.01% to about 10% (w/w); from about 1% to about 50% (w/w) of a medium chain triglyceride (MCT); and from about 50% to about 99% (w/w) of a semi-fluorinated alkane compound, wherein the nano-emulsion has a droplet particle size D90 of less than about 100 nm, less than about 20 nm, less than about 10 nm, less than about 5 nm, or from about 2 to about 3 nm.
37. The topical ophthalmological nano-emulsion of embodiment 36, wherein the semi-fluorinated alkane compound is selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).
38. The topical ophthalmological nano-emulsion of any one of embodiments 36 or 37, wherein the MCT is present in a concentration of about 5% (w/w) and the semi-fluorinated alkane compound is present in a concentration of about 95% (w/w), wherein the MCT is present in a concentration of about 10% (w/w) and the semi-fluorinated alkane compound is present in a concentration of about 90% (w/w), wherein the MCT is present in a concentration of about 15% (w/w) and the semi-fluorinated alkane compound is present in a concentration of about 85% (w/w), or wherein the MCT is present in a concentration of about 20% (w/w) and the semi-fluorinated alkane compound is present in a concentration of about 80% (w/w).
39. A topical ophthalmological nano-emulsion comprising: about 0.001% to about 1% (w/w) or about 0.0001% to about 10% (w/w) active pharmaceutical ingredient (API), wherein the API has a solubility in MCT of at least from about 0.01% to about 10% (w/w); about 10% to about 30% (w/w) medium chain triglyceride (MCT); and about 70% to about 90% (w/w) semi-fluorinated alkane compound selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10), wherein the nano-emulsion has a droplet particle size D90 of less than about 100 nm, less than about 20 nm, less than about 10 nm, less than about 5 nm, or from about 2 to about 3 nm.
40. The topical ophthalmological nano-emulsion of any one of embodiments 20-39, wherein the API is a muscarinic receptor antagonist.
41. The topical ophthalmological nano-emulsion of any one of embodiments 20-40, wherein the API is atropine, an atropine mimetic or analog, or a free base or salt form thereof.
42. The topical ophthalmological nano-emulsion of any one of embodiments 20-41, wherein the API is atropine free base.
43. The topical ophthalmological composition of any one of embodiments 20-39, wherein the API is atropine, an atropine mimetic or analog, or a free base or salt form thereof and is present in a concentration of from about 0.001% to about 1% (w/w).
44. The topical ophthalmological nano-emulsion of any one of embodiments 20-39, wherein the API is not a muscarinic receptor antagonist.
45. The topical ophthalmological nano-emulsion of any one of embodiments 20-39, wherein the API is not atropine, an atropine mimetic or analog, or a free base or salt form thereof.
46. A dispenser comprising: a container portion comprising an interior wall defining an interior volume and adapted to contact and store a liquid within at least a portion of the interior volume; a dispensing portion adapted to dispense one or more drops of a liquid out of the container portion; and any one of the compositions of embodiments 1-19, 77, 78, 80-82, 84, 85, 101-106, 112-116, 122-128, 134-155, 160-164, 203, 209-214, or 220-224, or the nano-emulsions of embodiments 20-45, 77, 78, 80-82, 84, 85, 101-106, 112-128, or 134-155, 160-164, 203, 209-214, or 220-224, or a composition comprising: about 0.001% to about 5% (w/w) or about 0.0001% to about 10% (w/w) active pharmaceutical ingredient (API), wherein the API has a solubility in MCT of at least from about 0.01% to about 10% (w/w) from about 1% to about 50% (w/w) of a medium chain triglyceride (MCT); and from about 50% to about 99% (w/w) of a semi-fluorinated alkane compound, contained within the interior volume.
47. The dispenser of embodiment 46, wherein the interior wall comprises less than about 10% of one or more of low density polyethylene (LDPE) or polypropylene (PP).
48. The dispenser of any one of embodiments 46-47, wherein the interior wall does not comprise low density polyethylene (LDPE) or polypropylene (PP).
49. The dispenser of any one of embodiments 46-48, wherein the interior wall comprises glass or polyethylene terephthalate.
50. The dispenser of any one of embodiments 46-49, wherein the interior wall consists essentially of glass or polyethylene terephthalate (PET).
51. The dispenser of any one of embodiments 46-50, wherein the concentration of the API remains more than 90% of the initial API concentration after storage in the dispenser for a period of at least 6 months, or wherein the concentration of the API remains more than 90% of the initial API concentration after storage in the dispenser for a period of at least 12 months or wherein the concentration of the API remains more than 90% of the initial API concentration after storage in the dispenser for at least a period of 24 months.
52. The dispenser of any one of embodiments 46-51, wherein the container portion and the dispensing portion each, independently, consist essentially of glass or polyethylene terephthalate (PET).
53. The dispenser of any one of embodiments 46-52, wherein the volume of the one or more drops each independently is from about 5 μl to about 40 μl.
54. The dispenser of any one of embodiments 46-53, wherein the composition comprises: from about 0.001% to about 1% (w/w) atropine, an atropine mimetic or analog, or a free base or salt form thereof, about 5% to about 30% (w/w) medium chain triglyceride (MCT); and about 70% to about 95% (w/w) semi-fluorinated alkane compound selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyl decane (F6H10).
55. A method for treating, slowing the progression of, or reducing one or more symptoms of an ocular condition in a subject comprising administering the topical ophthalmological composition of any one of embodiments 1-19, 77, 78, 80-82, 84, 85, 101-106, 112-116, 122-128, 134-155, 160-164, 203, 209-214, or 220-224, or the nano-emulsion of any one of embodiments 20-45, 77, 78, 80-82, 84, 85, 101-106, 112-128, 134-155, 160-164, 203, 209-214, or 220-224 to an eye of the subject.
56. The method of embodiment 55, wherein the ocular condition is selected from neurotrophic keratitis, glaucoma, elevated intraocular pressure, ocular hypertension, presbyopia, myopia, ocular rosacea, dry eye disease, meibomian gland dysfunction, blepharitis, allergic conjunctivitis, atopic keratoconjunctivitis, vernal keratoconjunctivitis, pterygium, pinguecula, corneal transplant rejection, graft versus host disease, ocular allergy, uveitis, anterior uveitis, Behcet's disease, Sjogren's syndrome, Stevens-Johnson syndrome, ocular cicatricial pemphigoid, chronic ocular surface inflammation caused by viral infection, herpes simplex keratitis, atopic conjunctivitis, Lyell's syndrome, neovascularization induced by viral, bacterial, fungal, or parasitic infection, contact lens induced neovascularization, ulceration, alkali burns, stem cell deficiency, neovascular glaucoma, Steven Johnson syndrome, tumor in the eye, aphakia, pseudophakia, astigmatism, blepharospasm, cataract, conjunctival diseases, corneal diseases, corneal ulcer, eyelid diseases, lacrimal apparatus diseases, pupil disorders, refractive disorders, strabismus.
57. A method of delivering an API to an eye of a subject comprising administering any one of the compositions of 1-19, 77, 78, 80-82, 84, 85, 101-106, 112-116, 122-128, 134-155, or 160-164, 203, 209-214, or 220-224, or the nano-emulsions of 20-45, 77, 78, 80-82, 84, 85, 101-106, 112-128, 134-155, or 160-164, 203, 209-214, or 220-224 to the eye of the subject.
58. The method of any one of embodiments 55-57, wherein the API is selected from alpha agonists, antibiotics, corticosteroids, anticholinergic agents, antiglaucoma agents, antihistamines, antivirals, anti-parasitics, pressure regulators, topical transient receptor potential melastatin 8 (TRPM8) agonists, inhibitors of inflammatory cell binding, cycloplegics/mydriatics, mast cell stabilizers, miotics, ophthalmic NSAIDs, cyclosporine, riboflavin 5′-phosphate ophthalmic compounds, and VEGF inhibitors and VEGF receptor inhibitors.
59. The method of any one of embodiments 55 to 58, wherein the topical ophthalmological composition or the nano-emulsion is absorbed transcorneally in the eye.
60. The method of any one of embodiments 55 to 58, wherein the topical ophthalmological composition or the nano-emulsion is selectively absorbed transcorneally in the eye as compared to periorbitally.
61. The method of any one of embodiments 55 to 60, wherein the topical ophthalmological composition is non-aqueous; optionally wherein the topical ophthalmological composition is an emulsion.
62. The method of any one of embodiments 55 to 61, wherein the topical ophthalmological composition is non-aqueous, and wherein the topical ophthalmological composition or the nano-emulsion is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, or reducing one or more symptoms of the ocular condition.
63. The method of any one of embodiments 55 to 62, wherein the topical ophthalmological composition or the nano-emulsion is administered to the subject every about 6 to about 8 hours or three times a day.
64. The method of any one of embodiments 55 to 63, wherein the API is in a free base form.
65. A method for slowing myopia progression in a subject or for relieving vitreous floater symptoms in a subject, comprising administering the nano-emulsion of any one of embodiments 20-45 to an eye of the subject, wherein the API is a muscarinic receptor antagonist.
66. The method of embodiment 65, wherein the API is selected from atropine, an atropine mimetic or analog, or a free base or salt form thereof.
67. The method of embodiment 65 or 66, wherein the API is in a free base form.
68. The method of any one of embodiments 65 to 67, wherein the nano-emulsion is absorbed transcorneally in the eye.
69. The method of any one of embodiments 65 to 67, wherein the nano-emulsion is selectively absorbed transcorneally in the eye as compared to periorbitally.
70. The method of any one of embodiments 65 to 69, wherein the nano-emulsion is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for slowing myopia progression in a subject or for relieving vitreous floater symptoms in a subject.
71. The method of any one of embodiments 65 to 70, wherein the nano-emulsion is administered to the subject every about 6 to about 8 hours or three times a day.
72. A method of making the topical ophthalmological nano-emulsion of any one of embodiments 20-45, 77, 78, 80-82, 84, 85, 101-106, or 112-121, comprising the steps of: (a) combining an ocular API with MCT to form an MCT solution; (b) combining said MCT solution with a semi-fluorinated alkane compound to form a mixture, wherein said mixture comprises from about 1% to about 50% said MCT solution and from about 50% to about 99% of said semi-fluorinated alkane compound; and (c) emulsifying said mixture and reducing droplet particle size D90 within said mixture to less than 100 nm by physical agitation selected from high speed stirring, vortexing, sonicating, heating and stirring, or homogenizing the mixture until the droplet size reaches droplet particle size D90 of less than 100 nm.
73. The method of embodiment 72, further comprising measuring the droplet particle size of the mixture during or following said physical agitation.
74. The method of embodiment 72 or 73, wherein reducing droplet particle size D90 within said mixture to less than 100 nm comprises reducing droplet particle size D90 within said mixture to less than 50 nm, less than 20 nm, less than 10 nm, less than 5 nm, or from about 2 to about 3 nm.
75. The method of any one of embodiments 72-74, wherein the API is selected from alpha agonists, antibiotics, corticosteroids, anticholinergic agents, antiglaucoma agents, antihistamines, antivirals, anti-parasitics, pressure regulators, topical transient receptor potential melastatin 8 (TRPM8) agonists, inhibitors of inflammatory cell binding, cycloplegics/mydriatics, mast cell stabilizers, miotics, ophthalmic NSAIDs, cyclosporine, riboflavin 5′-phosphate ophthalmic compounds, and VEGF inhibitors and VEGF receptor inhibitors.
76. A method for treating or reducing one or more symptoms of blepharitis, ocular rosacea, rosacea, or eyelid margin redness in a subject, or for improving eyelash health in a subject, or for treating or eradicating Demodex mites, lice, scabies, or bed bugs in a subject, or for treating or preventing a vector-borne disease in a subject, comprising administering the compositions of 1-19 or the nano-emulsions of 20-45 to the subject, to an eye of the subject, or to skin or hair of the subject, wherein the API is an anti-parasitic.
77. The composition of any one of embodiments 1-19, or the nano-emulsions of 20-45, or the method of any one of embodiments 55-58 or 76, wherein the API is an isooxazoline parasiticide or a formamidine parasiticide.
78. The composition of any one of embodiments 1-19, or the nano-emulsions of 20-45, or the method of any one of embodiments 55-58 or 76, wherein the API is one or more of fluralaner, sarolaner, lotilaner, afoxolaner, and/or fluxametamide, a derivative, analogue, enantiomer, racemic mixture, free base, enantiomerically pure composition, or L- and D-isomer thereof.
79. A method for treating, slowing the progression of, or reducing one or more symptoms of dry eye disease, meibomian gland disfunction, or inflammatory dry eye in a subject, comprising administering the composition of any one of embodiments 1-19 or the nano-emulsions of 20-45 to an eye of the subject, wherein the API is selected from one or more of lifitegrast, AR-15512 ((1R,2S,5R)—N-(4-methoxyphenyl)-5-methyl-2-(1-methylethyl) cyclohexanecarboxamide), cyclosporine, or derivatives, analogues, pharmaceutically acceptable salts, free base form, racemic mixtures, or diastereomers or enantiomers thereof.
80. The composition of any one of embodiments 1-19, or the nano-emulsions of 20-45, or the method of any one of embodiments 55-58 or 65, wherein the API is lifitegrast, or a derivative, analogue, pharmaceutically acceptable salt, free base form, racemic mixture, or diastereomer or enantiomer thereof.
81. The composition of any one of embodiments 1-19, or the nano-emulsions of 20-45, or the method of any one of embodiments 55-58 or 76, wherein the API is cyclosporine, or a derivative, analogue, pharmaceutically acceptable salt, free base form, racemic mixture, or diastereomer or enantiomer thereof.
82. The composition of any one of embodiments 1-19, or the nano-emulsions of 20-45, or the method of any one of embodiments 55-58 or 76, wherein the API is AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof.
83. A method for treating, slowing the progression of, or reducing one or more symptoms of glaucoma, ocular hypertension, or high fluid pressure in an eye of a subject, or a method of lowering fluid pressure in an eye of a subject, comprising administering the composition of any one of embodiments 1-19, or the nano-emulsions of 20-45, to an eye of the subject, wherein the API is a pressure regulator.
84. The composition of any one of embodiments 1-19, or the nano-emulsions of 20-45, or the method of any one of embodiments 55-58 or 76, wherein the API is selected from one or more of Brimonidine, Apraclonidine Iopidine, Dorzolamide, Brinzolamide, Azopt, Timolol, Timoptic/Istalol/Betimol, Betaxolol, Betoptic, Levobunolol, Betagan, Metipranolol, Optipranolol, Latanoprost, Xalatan Green, Bimatoprost, Lumigan, Tafluprost, Zioptan, Latanoprostene, Vyzulta Turquoise, Pilocarpine, Carbachol, Isopto Carbachol, Netarsudil, and Rhopressa, and pharmaceutically acceptable salts or free base forms thereof.
85. The composition of any one of embodiments 1-19, or the nano-emulsions of 20-45, or the method of any one of embodiments 55-58 or 76, wherein the API is Brimonidine.
86. A method for treating, slowing the progression of, or reducing one or more symptoms of an ocular condition in a subject comprising administering a topical ophthalmological composition to an eye of the subject, wherein the topical ophthalmological composition comprises: about 0.001% to about 5% (w/w) active pharmaceutical ingredient (API), wherein the API has a solubility in medium chain triglyceride (MCT) of at least from about 0.01% to about 10% (w/w), and wherein the API is atropine; from about 1% to about 50% (w/w) of an MCT; and from about 50% to about 99% (w/w) of a semi-fluorinated alkane compound; wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, or reducing one or more symptoms of the ocular condition; optionally wherein the topical ophthalmological composition is non-aqueous; and optionally wherein the API is in a free base form.
87. The method of embodiment 86, wherein the ocular condition is selected from neurotrophic keratitis, glaucoma, elevated intraocular pressure, ocular hypertension, presbyopia, myopia, ocular rosacea, dry eye disease, meibomian gland dysfunction, blepharitis, allergic conjunctivitis, atopic keratoconjunctivitis, vernal keratoconjunctivitis, pterygium, pinguecula, corneal transplant rejection, graft versus host disease, ocular allergy, uveitis, anterior uveitis, Behcet's disease, Sjogren's syndrome, Stevens-Johnson syndrome, ocular cicatricial pemphigoid, chronic ocular surface inflammation caused by viral infection, herpes simplex keratitis, atopic conjunctivitis, Lyell's syndrome, neovascularization induced by viral, bacterial, fungal, or parasitic infection, contact lens induced neovascularization, ulceration, alkali burns, stem cell deficiency, neovascular glaucoma, Steven Johnson syndrome, tumor in the eye, aphakia, pseudophakia, astigmatism, blepharospasm, cataract, conjunctival diseases, corneal diseases, corneal ulcer, eyelid diseases, lacrimal apparatus diseases, pupil disorders, refractive disorders, strabismus.
88. A method of delivering an active pharmaceutical ingredient (API) to an eye of a subject comprising administering to the eye of the subject a topical ophthalmological composition, wherein the topical ophthalmological composition comprises: about 0.001% to about 5% (w/w) API, wherein the API has a solubility in medium chain triglyceride (MCT) of at least from about 0.01% to about 10% (w/w), and wherein the API is atropine; from about 1% to about 50% (w/w) of an MCT; and from about 50% to about 99% (w/w) of a semi-fluorinated alkane compound; wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, or reducing one or more symptoms of the ocular condition; optionally wherein the topical ophthalmological composition is non-aqueous; and optionally wherein the API is in a free base form.
89. A method for slowing myopia progression in a subject or for relieving vitreous floater symptoms in a subject, comprising administering to an eye of the subject a topical ophthalmological composition, wherein the topical ophthalmological composition comprises: about 0.001% to about 5% (w/w) API, wherein the API has a solubility in medium chain triglyceride (MCT) of at least from about 0.01% to about 10% (w/w), and wherein the API is selected from atropine or an atropine mimetic; from about 1% to about 50% (w/w) of an MCT; and from about 50% to about 99% (w/w) of a semi-fluorinated alkane compound; wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for slowing myopia progression in a subject or for relieving vitreous floater symptoms in a subject; optionally wherein the topical ophthalmological composition is non-aqueous; and optionally wherein the API is in a free base form.
90. The method of any one of embodiments 86 to 89, wherein the topical ophthalmological composition is absorbed transcorneally in the eye.
91. The method of any one of embodiments 86 to 89, wherein the topical ophthalmological composition is selectively absorbed transcorneally in the eye as compared to periorbitally.
92. A method of administering to a subject a topical ophthalmological composition for selective transcorneal absorption, the method comprising administering to an eye of the subject the topical ophthalmological composition of any one of embodiments 1 to 19 or the topical ophthalmological nano-emulsion of any one of embodiments 20 to 45; optionally wherein the topical ophthalmological composition is non-aqueous; and optionally wherein the API is in free base form.
93. A method of treating an ocular condition in a subject, the method comprising: (a) (i) selecting a subject in need of transcorneal absorption of an API; or (ii) selecting a subject having an ocular condition, wherein the ocular condition is in need of transcorneal absorption of an API; and (b) administering to an eye of the subject the topical ophthalmological composition of any one of embodiments 1 to 19 or the topical ophthalmological nano-emulsion of any one of embodiments 20 to 45; optionally wherein the topical ophthalmological composition is non-aqueous; and optionally wherein the API is in free base form.
94. A method of treating an ocular condition in a subject, the method comprising: (a) (i) selecting a subject in need of transcorneal absorption of an active pharmaceutical ingredient (API), or (ii) selecting a subject having an ocular condition, wherein the ocular condition is in need of transcorneal absorption of an API; and (b) administering to an eye of the subject a topical ophthalmological composition, wherein the topical ophthalmological composition comprises: about 0.001% to about 5% (w/w) API, wherein the API has a solubility in medium chain triglyceride (MCT) of at least from about 0.01% to about 10% (w/w), and wherein the API is atropine; from about 1% to about 50% (w/w) of an MCT; and from about 50% to about 99% (w/w) of a semi-fluorinated alkane compound; optionally wherein the topical ophthalmological composition is non-aqueous; and optionally wherein the API is in a free base form.
95. A method of selectively delivering an API to a cornea of an eye of a subject, the method comprising administering to the eye of the subject the topical ophthalmological composition of any one of embodiments 1 to 19 or the topical ophthalmological nano-emulsion of any one of embodiments 20 to 45; optionally wherein the topical ophthalmological composition is non-aqueous; and optionally wherein the API is in free base form.
96. A method of selectively delivering an active pharmaceutical ingredient (API) to a cornea of an eye of a subject, the method comprising administering to the eye of the subject a topical ophthalmological composition, wherein the topical ophthalmological composition comprises: about 0.001% to about 5% (w/w) API, wherein the API has a solubility in medium chain triglyceride (MCT) of at least from about 0.01% to about 10% (w/w), and wherein the API is atropine; from about 1% to about 50% (w/w) of an MCT; and from about 50% to about 99% (w/w) of a semi-fluorinated alkane compound; optionally wherein the topical ophthalmological composition is non-aqueous; and optionally wherein the API is in a free base form; optionally wherein the topical ophthalmological composition is administered to the eye of the subject about every about 6 to about 8 hours or three times a day, and/or optionally wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating an ocular condition or symptom thereof.
97. A method of treating an ocular condition in a subject, the method comprising administering to a subject the topical ophthalmological composition of any one of embodiments 1 to 19 or the topical ophthalmological nano-emulsion of any one of embodiments 20 to 45, wherein the topical ophthalmological composition is non-aqueous; wherein the subject has failed to respond to treatment with an aqueous composition comprising the API, wherein the subject has had an adverse side effect from the API after receiving an aqueous composition comprising the API, and/or wherein the subject is highly sensitive to an aqueous composition comprising the API; optionally wherein the API is in free base form; optionally wherein the topical ophthalmological composition or the topical ophthalmological nano-emulsion is administered to the eye of the subject about every about 6 to about 8 hours or three times a day, and/or optionally wherein the topical ophthalmological composition or the topical ophthalmological nano-emulsion is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating the ocular condition.
98. A method of treating an ocular condition in a subject, the method comprising administering to a subject the topical ophthalmological composition of any one of embodiments 1 to 19 or the topical ophthalmological nano-emulsion of any one of embodiments 20 to 45, wherein the topical ophthalmological composition comprises: about 0.001% to about 5% (w/w) API, wherein the API has a solubility in medium chain triglyceride (MCT) of at least from about 0.01% to about 10% (w/w), and wherein the API is atropine; from about 1% to about 50% (w/w) of an MCT; and from about 50% to about 99% (w/w) of a semi-fluorinated alkane compound; wherein the topical ophthalmological composition is non-aqueous; wherein the subject has failed to respond to treatment with an aqueous composition comprising the API, wherein the subject has had an adverse side effect from the API after receiving an aqueous composition comprising the API, and/or wherein the subject is highly sensitive to an aqueous composition comprising the API; optionally wherein the API is in free base form; optionally wherein the topical ophthalmological composition is administered to the eye of the subject about every about 6 to about 8 hours or three times a day, and/or optionally wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating the ocular condition.
99. A method of formulating a topical ophthalmological composition for transcorneal absorption, the method comprising: (a) combining an active pharmaceutical ingredient (API) with medium chain triglyceride (MCT) to form an MCT solution; (b) combining said MCT solution with a semi-fluorinated alkane compound to form a mixture, wherein said mixture comprises from about 1% to about 50% said MCT solution and from about 50% to about 99% of said semi-fluorinated alkane compound; and (c) emulsifying said mixture and reducing droplet particle size D90 within said mixture to less than 100 nm by physical agitation selected from high speed stirring, vortexing, sonicating, heating and stirring, or homogenizing the mixture until the droplet size reaches droplet particle size D90 of less than 100 nm; wherein the topical ophthalmological composition for transcorneal absorption comprises about 0.001% to about 5% (w/w) API, from about 1% to about 50% (w/w) of the MCT, and from about 50% to about 99% (w/w) of the semi-fluorinated alkane compound, wherein the API has a solubility in medium chain triglyceride (MCT) of at least from about 0.01% to about 10% (w/w), and wherein the API is not atropine or a salt thereof, and wherein the topical ophthalmological composition is non-aqueous.
100. A method of formulating a topical ophthalmological composition for transcorneal absorption, the method comprising: (a) identifying an aqueous topical ophthalmological composition comprising an active pharmaceutical ingredient (API) for selective transcorneal absorption; (b) combining an API with medium chain triglyceride (MCT) and semi-fluorinated alkane compound to form a mixture; and (d) emulsifying said mixture; optionally wherein the topical ophthalmological composition for transcorneal absorption comprises about 0.001% to about 5% (w/w) API, from about 1% to about 50% (w/w) of the MCT, and from about 50% to about 99% (w/w) of the semi-fluorinated alkane compound, and optionally wherein the API has a solubility in medium chain triglyceride (MCT) of at least from about 0.01% to about 10% (w/w).
101. The method of embodiment 79 or the compositions, nano-emulsions, or methods of embodiment 80, wherein: the MCT is present in an amount of from about 1% to about 50% (w/w), optionally from about 1% to about 30% (w/w), from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
102. The methods, compositions, or nano-emulsions of embodiment 101, wherein the semi-fluorinated alkane compound is selected from perfluorobutylheptane (F4H5) and perfluorohexyloctane (F6H8).
103. The methods, compositions, or nano-emulsions of any one of embodiments 101-102, wherein the MCT is selected from a glyceryl tricaprylate/tricaprate and a glyceryl tricaprylate/tricaprylin.
104. The methods, compositions, or nano-emulsions of any one of embodiments 101-103, wherein the lifitegrast or a derivative, analogue, pharmaceutically acceptable salt, free base form, racemic mixture, or diastereomer or enantiomer thereof is present in the composition in an amount of from about 0.01 to about 10 mg/ml, optionally from about 0.01 to about 5 mg/ml, optionally from about 0.01 to about 1 mg/ml.
105. The methods, compositions, or nano-emulsions of any one of embodiments 101-103, wherein the lifitegrast or a derivative, analogue, pharmaceutically acceptable salt, free base form, racemic mixture, or diastereomer or enantiomer thereof is present in the composition in an amount of from about 0.05% to about 20% (w/w), optionally from about 0.05% to about 10% (w/w), optionally from about 0.05% to about 5% (w/w), optionally selected from about 0.05%, 0.1%, 0.5%, 1%, 2%, 3%, 4%, or 5% (w/w).
106. The compositions of any one of embodiments 1-19, the nano-emulsions of any one of embodiments 20-40 and 45, or the methods of any one of embodiments 65 and 86-100, wherein: the API is aceclidine, or a derivative, analogue, pharmaceutically acceptable salt, free base form, racemic mixture, or diastereomer or enantiomer thereof.
107. The methods of embodiment 106, wherein the ocular condition is presbyopia or myopia.
108. A method for treating, slowing the progression of, improving one or more symptoms of, myopia or presbyopia in a subject, comprising administering to an eye of the subject a topical ophthalmological composition, wherein the topical ophthalmological composition comprises: about 0.001% to about 10% (w/w) API, wherein the API is selected from aceclidine, or a derivative, analogue, pharmaceutically acceptable salt, free base form, racemic mixture, or diastereomer or enantiomer thereof, from about 1% to about 50% (w/w) of an MCT; and from about 50% to about 99% (w/w) of a semi-fluorinated alkane compound.
109. The method of embodiment 108, wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject; optionally wherein the topical ophthalmological composition is non-aqueous; and optionally wherein the API is in a free base form.
110. The method of any one of embodiments 108-109, wherein the topical ophthalmological composition is absorbed transcorneally in the eye.
111. The method of any one of embodiments 108-109, wherein the topical ophthalmological composition is selectively absorbed transcorneally in the eye as compared to periorbitally.
112. The compositions or nano-emulsions of embodiment 106, or the methods of any one of embodiments 106-111 wherein: the MCT is present in an amount of from about 1% to about 30% (w/w), optionally from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
113. The compositions or nano-emulsions of any one of embodiments 106 or 112, or the methods of any one of embodiments 106-112, wherein the semi-fluorinated alkane compound is selected from perfluorobutylheptane (F4H5) and perfluorohexyloctane (F6H8).
114. The compositions or nano-emulsions of any one of embodiments 106 or 112-113, or the methods of any one of embodiments 106-113, wherein the MCT is selected from a glyceryl tricaprylate/tricaprate and a glyceryl tricaprylate/tricaprylin.
115. The compositions or nano-emulsions of any one of embodiments 106 or 112-114, or the methods of any one of embodiments 106-114, wherein the aceclidine, or a derivative, analogue, pharmaceutically acceptable salt, free base form, racemic mixture, or diastereomer or enantiomer thereof is present in the composition in an amount of from about 1 to about 30 mg/ml, optionally from about 5 to about 20 mg/ml, optionally from about 10 to about 15 mg/ml.
116. The compositions or nano-emulsions of any one of embodiments 106 or 112-115, or the methods of any one of embodiments 106-115, wherein the aceclidine, or a derivative, analogue, pharmaceutically acceptable salt, free base form, racemic mixture, or diastereomer or enantiomer thereof is present in the composition in an amount of from about 0.1% to about 10% (w/w), optionally from about 1% to about 3% (w/w), optionally about 1.75% (w/w).
117. The nano-emulsions of embodiment 41 or the methods of any one of embodiments 66, 86-89, or 94, wherein: the MCT is present in an amount of from about 1% to about 30% (w/w), optionally from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
118. The nano-emulsions or methods of embodiment 117, wherein the semi-fluorinated alkane compound is selected from perfluorobutylheptane (F4H5) and perfluorohexyloctane (F6H8).
119. The nano-emulsions or methods of any one of embodiments 117-118, wherein the MCT is selected from a glyceryl tricaprylate/tricaprate and a glyceryl tricaprylate/tricaprylin.
120. The nano-emulsions or methods of any one of embodiments 117-119, wherein the atropine, atropine mimetic or analog, or a free base or salt form thereof is present in the composition in an amount of from about 0.005 to about 1 mg/ml, optionally from about 0.01 to about 0.75 mg/ml, optionally from about 0.01 to about 0.1 mg/ml, optionally from about 0.05 to about 0.75 mg/ml, optionally from about 0.01 to about 0.03 mg/ml, optionally from about 0.1 to about 0.5 mg/ml.
121. The nano-emulsions or methods of any one of embodiments 117-119, wherein the atropine, atropine mimetic or analog, or a free base or salt form thereof is present in the composition in an amount of from about 0.0005% to about 2% (w/w), optionally from about 0.001% to about 0.005%, 0.001% to about 0.01%, optionally from about 0.005% to about 0.5%, optionally from about 0.005% to about 0.1% (w/w), optionally from about 0.008% to about 0.02%, optionally about 0.01% (w/w).
122. The method of embodiment 79 or the compositions, nano-emulsions, or methods of embodiment 82, wherein: the MCT is present in an amount of from about 1% to about 50% (w/w), optionally from about 1% to about 30% (w/w), from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w), wherein the API is selected from one or more of AR-15512 ((1R,2S,5R)—N-(4-methoxyphenyl)-5-methyl-2-(1-methylethyl) cyclohexanecarboxamide), or derivatives, analogues, pharmaceutically acceptable salts, free base form, racemic mixtures, or diastereomers or enantiomers thereof.
123. The methods, compositions, or nano-emulsions of embodiment 122, wherein the semi-fluorinated alkane compound is selected from perfluorobutylheptane (F4H5) and perfluorohexyloctane (F6H8).
124. The methods, compositions, or nano-emulsions of any one of embodiments 122-123, wherein the MCT is selected from a glyceryl tricaprylate/tricaprate and a glyceryl tricaprylate/tricaprylin.
125. The methods, compositions, or nano-emulsions of any one of embodiments 122-123, wherein the MCT is selected from a coco-caprylate/caprate, a propylene glycol dicaprylocaprate, a glyceryl tricaprylate/tricaprate, and a glyceryl tricaprylate/tricaprylin.
126. The methods, compositions, or nano-emulsions of any one of embodiments 122-125, wherein the AR-15512 ((1R,2S,5R)—N-(4-methoxyphenyl)-5-methyl-2-(1-methylethyl) cyclohexanecarboxamide) or a derivative, analogue, racemic mixture, or diastereomer or enantiomer thereof is present in the composition in an amount of from about 0.001 mg/ml to 1 mg/ml, optionally 0.01 mg/ml to 1 mg/ml, 0.01 mg/ml to 0.1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, or about 0.1 mg/ml.
127. The methods, compositions, or nano-emulsions of any one of embodiments 122-125, wherein the AR-15512 ((1R,2S,5R)—N-(4-methoxyphenyl)-5-methyl-2-(1-methylethyl) cyclohexanecarboxamide) or a derivative, analogue, racemic mixture, or diastereomer or enantiomer thereof is present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).
128. The compositions of any one of embodiments 1-19, the nano-emulsions of any one of embodiments 20-40 and 45, or the methods of any one of embodiments 65 and 86-100, wherein: the API is AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof.
129. The method of embodiment 128, wherein the ocular condition is presbyopia or myopia.
130. A method for treating, slowing the progression of, improving one or more symptoms of, myopia or presbyopia in a subject, comprising administering to an eye of the subject a topical ophthalmological composition, wherein the topical ophthalmological composition comprises: about 0.0001% to about 0.1% (w/w) API, wherein the API is selected from AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof, from about 0.1% to about 50% (w/w) of an MCT; and from about 50% to about 99% (w/w) of a semi-fluorinated alkane compound.
131. The method of embodiment 130, wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject; optionally wherein the topical ophthalmological composition is non-aqueous; and optionally wherein the API is in a free base form.
132. The method of any one of embodiments 130-131, wherein the topical ophthalmological composition is absorbed transcorneally in the eye.
133. The method of any one of embodiments 130-131, wherein the topical ophthalmological composition is selectively absorbed transcorneally in the eye as compared to periorbitally.
134. The compositions or nano-emulsions of embodiment 128, or the methods of any one of embodiments 128-133, wherein: the MCT is present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
135. The compositions or nano-emulsions of any one of embodiments 128 or 134, or the methods of any one of embodiments 128-134, wherein the semi-fluorinated alkane compound is selected from perfluorobutylheptane (F4H5) and perfluorohexyloctane (F6H8).
136. The compositions or nano-emulsions of any one of embodiments 128 or 134-135, or the methods of any one of embodiments 128-135, wherein the MCT is selected from coco-caprylate/caprate, a propylene glycol dicaprylocaprate, a glyceryl tricaprylate/tricaprate, and a glyceryl tricaprylate/tricaprylin.
137. The compositions or nano-emulsions of any one of embodiments 128 or 134-136, or the methods of any one of embodiments 128-136, wherein the AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof is present in the composition in an amount of from about 0.001-1 mg/ml, optionally from about 0.01 mg/mL to 1 mg/ml.
138. The compositions or nano-emulsions of any one of embodiments 128 or 134-137, or the methods of any one of embodiments 128-137, wherein the AR-15512 ((1R,2S,5R)—N-(4-methoxyphenyl)-5-methyl-2-(1-methylethyl) cyclohexanecarboxamide), or a derivative, analogue, racemic mixture, or diastereomer or enantiomer thereof is present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).
139. The compositions of any one of embodiments 1-19, the nano-emulsions of any one of embodiments 20-39, 44, and 45, or the methods of any one of embodiments 76-78, wherein: the API is lotilaner, or a derivative, analogue, racemic mixture, or diastereomer or enantiomer thereof.
140. A method for aiding in eradication or reduction of parasite infestations of the eyes, ocular tissue, skin, eyelashes, or other body hair; for treating or reducing or relieving symptoms of certain diseases or conditions, such as ocular, skin, vector-borne, or other diseases cause by parasites; for treating or reducing or relieving symptoms of certain ocular diseases or conditions, such as blepharitis, meibomian gland disfunction, or rosacea caused by parasites such as Demodex mites; for treating or reducing or relieving symptoms of eyelid margin redness caused by Demodex mites, or improving eyelash health in a subject; for eradicating, reducing the population of, treating or reducing or relieving symptoms of lice, scabies, or bed bugs and associated skin, hair, scalp, or follicular conditions; the method comprising administering to a subject in need thereof a topical ophthalmological composition, wherein the topical ophthalmological composition comprises: about 0.0001% to about 10% (w/w) API, wherein the API is selected from lotilaner, or a derivative, analogue, racemic mixture, or diastereomer or enantiomer thereof, from about 0.1% to about 50% (w/w) of an MCT; and from about 50% to about 99% (w/w) of a semi-fluorinated alkane compound, optionally wherein the topical ophthalmological composition comprises about 0.25% (w/w) API.
141. The method of embodiment 140, wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject; optionally wherein the topical ophthalmological composition is non-aqueous; and optionally wherein the API is in a free base form.
142. The method of any one of embodiments 140-141, wherein the topical ophthalmological composition is absorbed transcorneally in the eye.
143. The method of any one of embodiments 140-141, wherein the topical ophthalmological composition is selectively absorbed transcorneally in the eye as compared to periorbitally.
144. The compositions or nano-emulsions of embodiment 139, or the methods of any one of embodiments 139-143, wherein: the MCT is present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
145. The compositions or nano-emulsions of any one of embodiments 139 or 144, or the methods of any one of embodiments 139-143, wherein the semi-fluorinated alkane compound is selected from perfluorobutylheptane (F4H5) and perfluorohexyloctane (F6H8).
146. The compositions or nano-emulsions of any one of embodiments 139 or 144-145, or the methods of any one of embodiments 139-145, wherein the MCT is selected from coco-caprylate/caprate, a propylene glycol dicaprylocaprate, a glyceryl tricaprylate/tricaprate, and a glyceryl tricaprylate/tricaprylin.
147. The compositions or nano-emulsions of any one of embodiments 139 or 144-146, or the methods of any one of embodiments 139-146, wherein the lotilaner, or a derivative, analogue, racemic mixture, or diastereomer or enantiomer thereof is present in the composition in an amount of from 0.001 mg/ml to 10 mg/ml, optionally 0.01 mg/ml to 5 mg/ml, 0.01 mg/ml to 4 mg/ml, optionally 0.1 mg/ml to 5 mg/ml, optionally 1 mg/ml to 5 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 1 mg/ml, about 1.5 mg/ml, about 2 mg/ml, about 2.5 mg/ml, about 3 mg/ml, about 3.2 mg/ml, about 3.5 mg/ml, about 4 mg/ml, about 4.5 mg/ml, or about 5 mg/ml; optionally wherein the lotilaner, or a derivative, analogue, racemic mixture, or diastereomer or enantiomer thereof is present in the composition in an amount of about 3.2 mg/ml.
148. The compositions or nano-emulsions of any one of embodiments 139 or 144-147, or the methods of any one of embodiments 139-147, wherein the lotilaner, or a derivative, analogue, racemic mixture, or diastereomer or enantiomer thereof is present in the composition in an amount of from about 0.0001% to 1% (w/w), optionally from about 0.001% to about 1% (w/w), optionally from about 0.0001% to 0.9%, optionally from about 0.0001% to 0.9%, optionally from about 0.0001% to 0.8%, optionally from about 0.0001% to 0.7%, optionally from about 0.0001% to 0.6%, optionally from about 0.0001% to 0.5%, optionally from about 0.0001% to 0.4%, optionally from about 0.0001% to 0.3%, optionally from about 0.001% to 0.9%, optionally from about 0.001% to 0.9%, optionally from about 0.001% to 0.8%, optionally from about 0.001% to 0.7%, optionally from about 0.001% to 0.6%, optionally from about 0.001% to 0.5%, optionally from about 0.001% to 0.4%, optionally from about 0.001% to 0.3%, optionally selected from about 0.01%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, or about 0.1% (w/w), optionally about 0.25% (w/w).
149. The method of embodiment 79 or the compositions, nano-emulsions, or methods of embodiment 81, wherein: the MCT is present in an amount of from about 1% to about 50% (w/w), optionally from about 1% to about 30% (w/w), from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
150. The methods, compositions, or nano-emulsions of embodiment 149, wherein the semi-fluorinated alkane compound is selected from perfluorobutylheptane (F4H5) and perfluorohexyloctane (F6H8).
151. The methods, compositions, or nano-emulsions of any one of embodiments 149-150, wherein the MCT is selected from a glyceryl tricaprylate/tricaprate and a glyceryl tricaprylate/tricaprylin.
152. The methods, compositions, or nano-emulsions of any one of embodiments 149-150, wherein the MCT is selected from a coco-caprylate/caprate, a propylene glycol dicaprylocaprate, a glyceryl tricaprylate/tricaprate, and a glyceryl tricaprylate/tricaprylin.
153. The methods, compositions, or nano-emulsions of any one of embodiments 149-152, wherein the cyclosporine, or a derivative, analogue, pharmaceutically acceptable salt, free base form, racemic mixture, or diastereomer or enantiomer thereof is present in the composition in an amount of from about 0.001 mg/ml to 10 mg/ml, optionally 0.01 mg/ml to 1 mg/ml, 0.01 mg/ml to 0.1 mg/ml, optionally about 0.01 mg/ml, about 0.02 mg/ml, 0.03 mg/ml, about 0.04 mg/ml, about 0.05 mg/ml, about 0.06 mg/ml, about 0.07 mg/ml, about 0.08 mg/ml, about 0.09 mg/ml, or about 0.1 mg/ml, optionally in an amount of about 0.65 mg/ml.
154. The methods, compositions, or nano-emulsions of any one of embodiments 149-152, wherein the cyclosporine, or a derivative, analogue, pharmaceutically acceptable salt, free base form, racemic mixture, or diastereomer or enantiomer thereof is present in the composition in an amount of from about 0.0001% to 10% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w), optionally in an amount of about 0.05% (w/w).
155. The compositions of any one of embodiments 1-19, the nano-emulsions of any one of embodiments 20-39, 44, and 45, or the methods of any one of embodiments 79 and 81, wherein: the API is cyclosporine, or a derivative, analogue, pharmaceutically acceptable salt, free base form, racemic mixture, or diastereomer or enantiomer thereof.
156. A method for treating, slowing the progression of, improving one or more symptoms of, dry eye syndrome or keratoconjunctivitis sicca, dry eye disease, inflammatory dry eye, redness, meibomian gland disfunction, myopia or presbyopia in a subject, comprising administering to an eye of the subject a topical ophthalmological composition, wherein the topical ophthalmological composition comprises: about 0.0001% to about 0.1% (w/w) API, wherein the API is selected from cyclosporine, or a derivative, analogue, pharmaceutically acceptable salt, free base form, racemic mixture, or diastereomer or enantiomer thereof, from about 0.1% to about 50% (w/w) of an MCT; and from about 50% to about 99% (w/w) of a semi-fluorinated alkane compound.
157. The method of embodiment 156, wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject; optionally wherein the topical ophthalmological composition is non-aqueous; and optionally wherein the API is in a free base form.
158. The method of any one of embodiments 156-157, wherein the topical ophthalmological composition is absorbed transcorneally in the eye.
159. The method of any one of embodiments 156-158, wherein the topical ophthalmological composition is selectively absorbed transcorneally in the eye as compared to periorbitally.
160. The compositions or nano-emulsions of embodiment 155, or the methods of any one of embodiments 155-159, wherein: the MCT is present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
161. The compositions or nano-emulsions of any one of embodiments 155 or 160, or the methods of any one of embodiments 155-160, wherein the semi-fluorinated alkane compound is selected from perfluorobutylheptane (F4H5) and perfluorohexyloctane (F6H8).
162. The compositions or nano-emulsions of any one of embodiments 155 or 160-161, or the methods of any one of embodiments 155-161, wherein the MCT is selected from coco-caprylate/caprate, a propylene glycol dicaprylocaprate, a glyceryl tricaprylate/tricaprate, and a glyceryl tricaprylate/tricaprylin.
163. The compositions or nano-emulsions of any one of embodiments 155 or 160-162, or the methods of any one of embodiments 155-162, wherein the cyclosporine, or a derivative, analogue, pharmaceutically acceptable salt, free base form, racemic mixture, or diastereomer or enantiomer thereof is present in the composition in an amount of from about 0.001-1 mg/ml, optionally from about 0.01 mg/mL to 1 mg/ml.
164. The compositions or nano-emulsions of any one of embodiments 155 or 161-163, or the methods of any one of embodiments 155-163, wherein the cyclosporine, or a derivative, analogue, pharmaceutically acceptable salt, free base form, racemic mixture, or diastereomer or enantiomer thereof is present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).
165. A topical ophthalmological composition comprising: a therapeutically effective amount of a muscarinic cholinergic receptor agonist as an active pharmaceutical ingredient; and a semifluorinated alkane compound.
166. The topical ophthalmological composition of embodiment 165, wherein the semifluorinated alkane compound is present as a liquid vehicle.
167. The topical ophthalmological composition of any one of embodiment 165 or embodiment 166, wherein the muscarinic cholinergic receptor agonist is selected from the group consisting of aceclidine, pilocarpine, bethanechol, cevimeline, methacholine, xanomeline, and aprolidine.
168. The topical ophthalmological composition of any one of embodiments 165 to 167, wherein the semifluorinated alkane is a compound of formula RFRH or of formula RFRHRF, wherein RF is a perfluorinated hydrocarbon with 15 or less carbon atoms, and wherein RH is a nonfluorinated hydrocarbon with 15 or less carbon atoms.
169. The topical ophthalmological composition of any one of embodiments 165 to 168, wherein the semifluorinated alkane is selected from the group consisting of perfluorobutylheptane (F4H5), perfluorobutylhexane (F4H6), perfluorohexylbutane (F6H4), perfluorohexylhexane (F6H6), perfluorohexyloctane (F6H8), and perfluorohexyldecane (F6H10).
170. The topical ophthalmological composition of embodiment 169, wherein the semifluorinated alkane is F6H8 (perfluorohexyloctane).
171. The topical ophthalmological composition of embodiment 169, wherein the semifluorinated alkane is perfluorobutylheptane (F4H5).
172. The topical ophthalmological composition of any one of embodiments 165 to 171, further comprising one or more organic cosolvents.
173. The topical ophthalmological composition of embodiment 172, wherein the organic cosolvent is selected from the group consisting of medium-chain triglycerides (MCT), liquid paraffin, vitamin E acetate, D-a-tocopherol, oleic acid, ethyl oleate and combinations thereof.
174. The topical ophthalmological composition of embodiment 172, wherein the organic cosolvent is medium-chain triglycerides (MCT) or medium-chain triglycerides (MCT) having two or three fatty acids each independently having an aliphatic tail of 6-12 carbon atoms.
175. The topical ophthalmological composition of any one of embodiments 172 to 174, wherein the concentration of the organic cosolvent in the composition is less than or equal to about 50% (w/w).
176. The topical ophthalmological composition of any one of embodiments 172 to 174, wherein the concentration of organic cosolvent in the composition is from about 1% to 15% (w/w).
177. The topical ophthalmological composition of any one of embodiments 172 to 174, wherein the organic cosolvent is present in a concentration of from about 1% (w/w) to about 50% (w/w) and the semi-fluorinated alkane compound is present in a concentration of from about 50% to about 99%.
178. The topical ophthalmological composition of any one of embodiments 165 to 177, wherein the muscarinic cholinergic receptor agonist is present in a concentration of from about 0.1% to about 2.0% (w/w), 0.5% to about 2.0% (w/w), or 0.5% to about 1.5% (w/w).
179. The topical ophthalmological composition of any one of embodiments 165 to 178, wherein the muscarinic cholinergic receptor agonist is aceclidine.
180. The topical ophthalmological composition of embodiment 179, wherein the aceclidine is in a free base form or a salt form.
181. The topical ophthalmological composition of embodiment 180, wherein the aceclidine is in free base form and the concentration of the aceclidine is from about 0.1% to about 2.0% (w/w), from about 0.5% to about 2.0% (w/w), or from about 0.5% to about 1.5% (w/w).
182. The topical ophthalmological composition of any one of embodiments 165 to 178, wherein the muscarinic cholinergic receptor agonist is pilocarpine.
183. The topical ophthalmological composition of embodiment 182, wherein the pilocarpine is in a free base form or a salt form.
184. The topical ophthalmological composition of embodiment 180, wherein the pilocarpine is in free base form and the concentration of the pilocarpine is from about 0.1% to about 2.0% (w/w), from about 0.5% to about 2.0% (w/w), or from about 0.5% to about 1.5% (w/w).
185. The topical ophthalmological composition of any one of embodiments 165 to 184, wherein the topical ophthalmological composition is a non-aqueous solution, a suspension, or an emulsion.
186. The topical ophthalmological composition of any one of embodiments 165 to 185, wherein the muscarinic cholinergic receptor agonist in the topical ophthalmological composition is chemically stable at room temperature for at least 0.5 year, for at least 1 year, or for at least 2 years.
187. Eye drops comprising the topical ophthalmological composition of any one of embodiments 165 to 186.
188. The topical ophthalmological composition of any one of embodiments 165 to 186 or the eye drops of embodiment 187, wherein the topical ophthalmological composition causes minimal or no irritation in a mammalian eye.
189. A method for treating presbyopia in a subject in need thereof, comprising administering the topical ophthalmological composition of any one of embodiments 165 to 186 or the eye drops of embodiment 187 to an eye of the subject.
190. The method of embodiment 189, wherein the topical ophthalmological composition causes minimal or no irritation in the eye of the subject.
191. A topical ophthalmological composition comprising: a therapeutically effective amount of aceclidine; and a semi-fluorinated alkane compound as liquid vehicle.
192. The topical ophthalmological composition of embodiment 191, further comprising medium-chain triglycerides (MCT) having two or three fatty acids each independently having an aliphatic tail of 6-12 carbon atoms.
193. The topical ophthalmological composition of any one of embodiment 191 or 192, wherein the aceclidine is in free base form and is present in a concentration of from about 0.1% to about 2.0% (w/w), from about 0.5% to about 2.0% (w/w), or from about 0.5% to about 1.5% (w/w).
194. A topical ophthalmological composition comprising: a therapeutically effective amount of pilocarpine; and a semi-fluorinated alkane compound as liquid vehicle.
195. The topical ophthalmological composition of embodiment 194, further comprising medium-chain triglycerides (MCT) having two or three fatty acids each independently having an aliphatic tail of 6-12 carbon atoms.
196. The topical ophthalmological composition of any one of embodiment 194 or 195, wherein the pilocarpine is in free base form and is present in a concentration of from about 0.1% to about 2.0% (w/w), from about 0.5% to about 2.0% (w/w), or from about 0.5% to about 1.5% (w/w).
197. A method for treating glaucoma in a subject in need thereof, comprising administering the topical ophthalmological composition of any one of embodiments 165 to 186 or the eye drops of embodiment 187 to an eye of the subject.
198. The method of embodiment 197, wherein the topical ophthalmological composition causes minimal or no irritation in the eye of the subject.
199. A method for improving the ability of a subject to focus on a near object, comprising administering the topical ophthalmological composition of any one of embodiments 165 to 186 or the eye drops of embodiment 187 to an eye of the subject, wherein the subject has presbyopia.
200. The method of embodiment 199, wherein the topical ophthalmological composition causes minimal or no irritation in the eye of the subject.
201. A method for reducing intraocular pressure in an eye of a subject in need thereof, comprising administering the topical ophthalmological composition of any one of embodiments 165 to 186 or the eye drops of embodiment 186 to the eye of the subject.
202. The method of embodiment 201, wherein the topical ophthalmological composition causes minimal or no irritation in the eye of the subject.
203. The compositions of any one of embodiments 1-19, the nano-emulsions of any one of embodiments 20-39, 44, and 45, or the methods of any one of embodiments 86 and 87, wherein: the API is ivermectin or a derivative, analogue, racemic mixture, or diastereomer or enantiomer thereof, optionally wherein the ivermectin is a mixture of ivermectin B1a and ivermectin B1b.
204. The method of embodiment 203, wherein the ocular condition is presbyopia or myopia.
205. A method for treating, slowing the progression of, improving one or more symptoms of, demodex blepharitis, for aiding in eradication or reduction of parasite infestations of the eyes, ocular tissue, skin, eyelashes, or other body hair; for treating or reducing or relieving symptoms of certain diseases or conditions, such as ocular, skin, vector-borne, or other diseases cause by parasites; for treating or reducing or relieving symptoms of certain ocular diseases or conditions, such as blepharitis, meibomian gland disfunction, or rosacea caused by parasites such as Demodex mites; for treating or reducing or relieving symptoms of eyelid margin redness caused by Demodex mites, or improving eyelash health in a subject; for eradicating, reducing the population of, treating or reducing or relieving symptoms of lice, scabies, or bed bugs and associated skin, hair, scalp, or follicular conditions; for treating, slowing the progression of, improving one or more symptoms of myopia or presbyopia in a subject, comprising administering to an eye of the subject a topical ophthalmological composition, wherein the topical ophthalmological composition comprises: about 0.0001% to about 0.5% (w/w) API, wherein the API is selected from ivermectin or a derivative, analogue, racemic mixture, or diastereomer or enantiomer thereof, optionally wherein the ivermectin is a mixture of ivermectin B1a and ivermectin B1b; from about 0.1% to about 50% (w/w) of an MCT; and from about 50% to about 99% (w/w) of a semi-fluorinated alkane compound, optionally wherein the topical ophthalmological composition comprises about 0.01% to 5% API or from 0.1 mg/ml to 50 mg/mL API.
206. The method of embodiment 205, wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject; optionally wherein the topical ophthalmological composition is non-aqueous; and optionally wherein the API is in a free base form.
207. The method of any one of embodiments 205-206, wherein the topical ophthalmological composition is absorbed transcorneally in the eye.
208. The method of any one of embodiments 205-206, wherein the topical ophthalmological composition is selectively absorbed transcorneally in the eye as compared to periorbitally.
209. The compositions or nano-emulsions of embodiment 203, or the methods of any one of embodiments 203-208, wherein: the MCT is present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
210. The compositions or nano-emulsions of any one of embodiments 203 or 209, or the methods of any one of embodiments 203-209, wherein the semi-fluorinated alkane compound is selected from perfluorobutylheptane (F4H5) and perfluorohexyloctane (F6H8).
211. The compositions or nano-emulsions of any one of embodiments 203 or 209-210, or the methods of any one of embodiments 203-210, wherein the MCT is selected from coco-caprylate/caprate, a propylene glycol dicaprylocaprate, a glyceryl tricaprylate/tricaprate, and a glyceryl tricaprylate/tricaprylin.
212. The compositions or nano-emulsions of any one of embodiments 203 or 209-211, or the methods of any one of embodiments 203-210, wherein the ivermectin or a derivative, analogue, racemic mixture, or diastereomer or enantiomer thereof, is present in the composition in an amount of from about 0.001-1 mg/ml, optionally from about 0.01 mg/mL to 1 mg/ml.
213. The compositions or nano-emulsions of any one of embodiments 203 or 209-211, or the methods of any one of embodiments 203-212, wherein the ivermectin or a derivative, analogue, racemic mixture, or diastereomer or enantiomer thereof is present in the composition in an amount of from about 0.0001% to 0.5% (w/w), optionally from about 0.01% to about 5% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).
214. The compositions of any one of embodiments 1-19, the nano-emulsions of any one of embodiments 20-39, 44, and 45, or the methods of any one of embodiments 86 and 87, wherein: the API is axitinib, an axitinib mimetic or analog, or a free base or salt form thereof.
215. The method of embodiment 214, wherein the ocular condition is presbyopia or myopia.
216. A method for treating, slowing the progression of, improving one or more symptoms of, myopia presbyopia, pinguecula, pterygium, or an ocular condition associated with angiogenesis (e.g., hyperemia, neovascularization, pterygium, pinguecula, glaucoma filtration surgery and minimally invasive glaucoma surgery (MIGS), cornea transplant surgery with graft rejection, graft versus host disease, dry eye disease, atopic conjunctivitis, rosacea, ocular pemphigoid, Lyell's syndrome, Steven Johnson syndrome, viral infection (e.g. HSV-1), bacterial infection, fungal infection, parasitic infection, contact lens induced neovascularization, ulceration, alkali burns, and stem cell deficiency), in a subject, comprising administering to an eye of the subject a topical ophthalmological composition, wherein the topical ophthalmological composition comprises: about 0.0001% to about 0.1% (w/w) API, wherein the API is selected from axitinib, an axitinib mimetic or analog, or a free base or salt form thereof, from about 0.1% to about 50% (w/w) of an MCT; and from about 50% to about 99% (w/w) of a semi-fluorinated alkane compound, optionally wherein the topical ophthalmological composition comprises about 0.5-1 mg/ml or about 0.05-0.1% (w/w) API.
217. The method of embodiment 216, wherein (i) the topical ophthalmological composition is administered to the eye of the subject every about 6 to about 8 hours or three times a day, or (ii) the topical ophthalmological composition is non-aqueous, and wherein the topical ophthalmological composition is administered at a dose 5-10%, 5-15%, 5-20%, 5-25%, 5-30%, 10-30%, 10-40%, 10-50%, 10-60%, 10-70%, 20-30%, 20-40%, 20-50%, 20-60%, 20-70%, 30-40%, 30-50%, 30-60%, 30-70%, 40-50%, 40-60%, 40-70%, or 50-70% less than a dose of an aqueous topical ophthalmological composition comprising the API for treating, slowing the progression of, improving one or more symptoms of, myopia or presbyopia in a subject; optionally wherein the topical ophthalmological composition is non-aqueous; and optionally wherein the API is in a free base form.
218. The method of any one of embodiments 216-217, wherein the topical ophthalmological composition is absorbed transcorneally in the eye.
219. The method of any one of embodiments 216-217, wherein the topical ophthalmological composition is selectively absorbed transcorneally in the eye as compared to periorbitally.
220. The compositions or nano-emulsions of embodiment 214, or the methods of any one of embodiments 214-219, wherein: the MCT is present in an amount of from about 0.1% to about 50% (w/w), optionally from about 0.1% to about 10% (w/w), optionally from about 1% to about 30% (w/w), optionally from about 5% to about 20% (w/w); optionally selected from about 5%, about 10%, about 15%, or about 20% (w/w); and the semi-fluorinated alkane compound is present in an amount of from about 70% to about 99% (w/w), optionally from about 80% to about 95% (w/w), optionally selected from about 80%, 85%, 90%, or 95% (w/w).
221. The compositions or nano-emulsions of any one of embodiments 214 or 220, or the methods of any one of embodiments 214-220, wherein the semi-fluorinated alkane compound is selected from perfluorobutylheptane (F4H5) and perfluorohexyloctane (F6H8).
222. The compositions or nano-emulsions of any one of embodiments 214 or 220-221, or the methods of any one of embodiments 214-221, wherein the MCT is selected from coco-caprylate/caprate, a propylene glycol dicaprylocaprate, a glyceryl tricaprylate/tricaprate, and a glyceryl tricaprylate/tricaprylin.
223. The compositions or nano-emulsions of any one of embodiments 214 or 220-222, or the methods of any one of embodiments 214-221, wherein the axitinib, axitinib mimetic or analog, or a free base or salt form thereof, is present in the composition in an amount of from about 0.001-1 mg/ml, optionally from about 0.01 mg/mL to 1 mg/ml, optionally from about 0.5-1 mg/ml.
224. The compositions or nano-emulsions of any one of embodiments 214 or 220-222, or the methods of any one of embodiments 214-223, wherein the axitinib, axitinib mimetic or analog, or a free base or salt form thereof is present in the composition in an amount of from about 0.0001% to 0.1% (w/w), optionally from about 0.001% to about 0.1% (w/w), optionally from about 0.001% to about 0.01% (w/w), optionally selected from about 0.001%, about 0.002%, about 0.003%, about 0.004%, about 0.005%, about 0.006%, about 0.007%, about 0.008%, about 0.009%, or about 0.01% (w/w).

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

### REFERENCES

Wang L., Syn N., Li S. etc, The penetration and distribution of topical atropine in animal ocular tissues, Acta Pharmacologica, 2019.
Berton B., Chennel P., Yessad M. etc, Stability of Ophthalmic Atropine Solutions for Child Myopia Control, Pharmaceutics 2020, 12, 781
Mashkovskii, M. D. “The relation between the chemical structure and pharmacological activity of some esters of 3-hydroxyquinuclidine (quinuclidine-3-ol)”, Proc. Intern. Pharmacol. Meeting, 1st, Stockholm, 1961, 7, 359-366.
Romano, John H. “Double-blind cross-over comparison of aceclidine and pilocarpine in open-angle glaucoma”, Brit. J. Ophthal. 1970 54, 510-521.
Cho, A. K., Jenden, D. J. and Lamb, S. I. “Rates of Alkaline Hydrolysis and Muscarinic Activity of Some Aminoacetates and Their Quaternary Ammonium Analogs”, J. of Medicinal Chem. 1972, 15, 391-394.
Grzybowski, A. et al “A Review of Pharmacological Presbyopia Treatment”, Asia-Pacific Journal of Ophthalmology, 2020, 9, 226-233.
Zhu, L. et al “Aceclidine and pilocarpine interact differently with muscarinic receptor in isolated rabbit iris muscle”, Life Sciences 2006, 78, 1617-1623.

1.-24. (canceled)

25. A topical ophthalmological composition comprising: a therapeutically effective amount of a muscarinic cholinergic receptor agonist as an active pharmaceutical ingredient; and a semi-fluorinated alkane compound, wherein the muscarinic cholinergic receptor agonist is selected from the group consisting of aceclidine, pilocarpine, bethanechol, cevimeline, methacholine, xanomeline, and aprolidine, or a derivative, analogue, pharmaceutically acceptable salt, free base form, racemic mixture, or diastereomer or enantiomer thereof.

26. The topical ophthalmological composition of claim 25, wherein the semi-fluorinated alkane compound is selected from the group consisting of perfluorobutylpentane (F4H5), perfluorobutylhexane (F4H6), perfluorohexylbutane (F6H4), perfluorohexylhexane (F6H6), perfluorohexyloctane (F6H8), and perfluorohexyl decane (F6H10).

27. The topical ophthalmological composition of claim 25, further comprising one or more organic cosolvents.

28. The topical ophthalmological composition of claim 27, wherein the organic cosolvent is selected from the group consisting of medium-chain triglycerides (MCT), liquid paraffin, vitamin E acetate, D-a-tocopherol, oleic acid, ethyl oleate and combinations thereof.

29. The topical ophthalmological composition of claim 28, wherein the medium-chain triglycerides (MCT) are medium-chain triglycerides (MCT) having two or three fatty acids each independently having an aliphatic tail of 6-12 carbon atoms.

30. The topical ophthalmological composition of claim 27, wherein the organic cosolvent is present in a concentration of from about 1% to about 20% (w/w).

31. The topical ophthalmological composition of claim 27, wherein the organic cosolvent is present in a concentration of from about 1% to about 20% (w/w) and the semi-fluorinated alkane compound is present in a concentration of from about 80% to about 99% (w/w).

32. The topical ophthalmological composition of claim 25, wherein the muscarinic cholinergic receptor agonist is present in a concentration of from about 0.5% to about 2.0% (w/w).

33. The topical ophthalmological composition of claim 28, wherein the organic cosolvent is medium-chain triglycerides (MCT).

34. The topical ophthalmological composition of claim 25, wherein the semi-fluorinated alkane compound is perfluorohexyloctane (F6H8).

35. The topical ophthalmological composition of claim 25, wherein the muscarinic cholinergic receptor agonist is aceclidine, or a derivative, analogue, pharmaceutically acceptable salt, free base form, racemic mixture, or diastereomer or enantiomer thereof.

36. The topical ophthalmological composition of claim 25, wherein the muscarinic cholinergic receptor agonist is aceclidine.

37. The topical ophthalmological composition of claim 25, wherein the composition is non-aqueous.

38. The topical ophthalmological composition of claim 25, wherein the composition is in the form of an eye drop.

39. A topical ophthalmological composition comprising:
from about 0.5% to about 2.0% (w/w) of a muscarinic cholinergic receptor agonist selected from the group consisting of aceclidine, pilocarpine, bethanechol, cevimeline, methacholine, xanomeline, and aprolidine, or a derivative, analogue, pharmaceutically acceptable salt, free base form, racemic mixture, or diastereomer or enantiomer thereof,
from about 80% to about 99% (w/w) of a semi-fluorinated alkane compound selected from the group consisting of perfluorobutylpentane (F4H5), perfluorobutylhexane (F4H6), perfluorohexylbutane (F6H4), perfluorohexylhexane (F6H6), perfluorohexyloctane (F6H8), and perfluorohexyl decane (F6H10); and
from about 1% to about 20% (w/w) of an organic cosolvent, wherein the organic cosolvent is medium-chain triglycerides (MCT).

40. A method for treating presbyopia in a subject in need thereof, comprising administering the topical ophthalmological composition of claim 38 to an eye of the subject.

41. A method for treating, slowing the progression of, or reducing one or more symptoms of presbyopia in a subject comprising administering the topical ophthalmological composition of claim 25 to an eye of the subject.
